(19) United States
(12) Patent Application Publication
(10) Pub. No.: US 2022/0008242 A1
(43) Pub. Date: Jan. 13, 2022

(54) Methods of Reducing Sleep Disordered Breathing and Structures Formed Therapy

(71) Applicants: Robert Graham Kitchell, Florence, AZ (US); Devin Matthew Cunning, Lake Havasu City, AZ (US); James Keith Lawson, Oklahoma City, OK (US); Mitchell Evan Berman, Valley village, CA (US)

(72) Inventors: Robert Graham Kitchell, Florence, AZ (US); Devin Matthew Cunning, Lake Havasu City, AZ (US); James Keith Lawson, Oklahoma City, OK (US); Mitchell Evan Berman, Valley village, CA (US)

(21) Appl. No.: 16/873,795

(22) Filed: Jul. 10, 2020

(51) Int. Cl. A61F 5/56; G16H 20/30; G16H 40/67; G16H 70/20; G16H 50/20; G16H 50/70; G16H 10/60; G16H 10/20; A61H 23/02; A61N 1/05; A61M 16/00; A61N 5/06; A61M 21/02; A61L 2/26; A61L 2/10

(52) U.S. Cl. CPC A61F 5/566; G16H 20/40; G16H 40/67; G16H 70/20; G16H 50/20; G16H 50/70; G16H 10/60; G16H 10/20; A61H 23/02; A61N 1/0548; A61M 16/0057; A61N 5/0603; A61M 21/02; A61L 2/26; A61L 2/10; G16H 20/30

(57) ABSTRACT

An oral therapy device structure and methods of utilizing said device are described. Embodiments of the oral therapy device structure include a top member and a bottom member, where the top member fits over at least a portion of the upper teeth of a user, and includes one or more partially embedded sensors. The bottom member fits over at least a portion of the bottom teeth of the user. A coupling structure physically joins a portion of the top member to a portion of the bottom member. A mandibular positioning drive (MPD) is at least partially embedded within the bottom member, where the MPD is capable of moving the bottom member from a first position to a second position.

### BACKGROUND

Forty-five (45) million Americans suffer from some form of Sleep Disordered Breathing (SDB). The presence of SDB disrupts sleep and significantly diminishes the human body's ability to repair itself and to perform other tasks needed for good health. Snoring is a common symptom of SDB. In and of itself snoring represents a minimal health risk. However, snoring can be an early indicator and precursor of obstructive sleep apnea (“OSA”), a serious medical condition. Those individuals whose snoring is accompanied by OSA require timely effective treatment to control symptoms and must be periodically monitored to assess disease state. The incidence of sleep apnea is directly linked to the four most costly diseases that afflict the American populace; heart disease, diabetes, obesity and cancer. Eighty-five percent (85%) of OSA patients are currently undiagnosed and untreated. OSA is defined as an episode in which an individual's upper airway (oropharynx) becomes obstructed and may totally (or at least partially) close during sleep causing temporary cessation of breathing. This stoppage in breathing substantially reduces airflow to the lungs, causing a drop in blood oxygen concentration resulting in serious cardiovascular distress and sleep fragmentation to the individual. Typically, the individual reacts to this suffocating condition by subconsciously arousing from sleep, causing the upper airway muscles to contract and re-open the airway. The reason why so many individuals suffer from SDB is that the upper airways of human beings lack rigid support and are held open only by active contraction of the airway muscles.

There are several therapeutic devices existing today that attempt to mitigate the symptoms of SDB resulting from airway obstruction. One such device is a continuous positive air pressure (CPAP) device. A CPAP device pumps air down the user's airway acting as a pneumatic splint to maintain patency of the airway during sleep. Some CPAP devices use either a nasal or an oral mask that is affixed over the face to maintain necessary air pressure seal. User compliance with CPAP devices approximates 50% due to a number of reasons including, mask discomfort, claustrophobic sensation, nasal and throat dryness and other factors. Surgical intervention comprising uvulopalatopharyngioplasty (UPPP), insertion of hypoglossal nerve stimulation apparatus, and other procedures can be invasive and costly and reimbursement by insurance carriers is usually approved only after less invasive interventions are unsuccessful.

### BRIEF DESCRIPTION OF THE DRAWINGS

While the specification concludes with claims particularly pointing out and distinctly claiming certain embodiments, the advantages of these embodiments can be more readily ascertained from the following description of the embodiments when read in conjunction with the accompanying drawings in which:

FIG. 1a represents a cross sectional view of an oral therapy device according to embodiments.

FIG. 1b represents a cross sectional view of an oral therapy device according to embodiments.

FIG. 1c represents a cross sectional view of an oral therapy device according to embodiments.

FIG. 1d represents a side view of an oral therapy device according to embodiments.

FIG. 1e represents a cross sectional view of a mandibular positioning drive according to embodiments.

FIG. 1f represents a cross sectional view of a mandibular positioning drive according to embodiments.

FIG. 1g represents a cross sectional view of a mandibular positioning drive according to embodiments.

FIG. 1h represents a cross sectional view of a mandibular positioning drive according to embodiments.

FIG. 2 represents a cross sectional view of an oral therapy device according to embodiments.

FIG. 3a represents a cross sectional view of an oral therapy device according to embodiments.

FIG. 3b represents a cross sectional view of an oral therapy device according to embodiments.

FIG. 3c represents a cross sectional view of an oral therapy device according to embodiments.

FIG. 3d represents a cross sectional view of an oral therapy device according to embodiments.

FIG. 3e represents a top view of an oral therapy device according to embodiments.

FIG. 3f represents a cross sectional view of an oral therapy device according to embodiments.

FIG. 3g represents a cross sectional view of an oral therapy device according to embodiments.

FIG. 3h represents a cross sectional view of an oral therapy device according to embodiments.

FIG. 3i represents a cross sectional view of an oral therapy device according to embodiments.

FIG. 3j represents a cross sectional view of an oral therapy device according to embodiments.

FIG. 3k represents a close-up cross-sectional view of anatomic structures according to embodiments.

FIG. 3l represents a cross sectional view of an oral therapy device according to embodiments.

FIG. 3m represents a cross sectional view of an oral therapy device according to embodiments.

FIG. 3n represents a cross sectional view of an oral therapy device according to embodiments.

FIG. 4 represents a cross sectional view of an oral therapy device according to embodiments.

FIG. 5a represents a side view of user and collaborative sensors according to embodiments.

FIG. 5b represents a side view of a user and collaborative sensor according to embodiments.

FIG. 6 represents a flowchart of basic operational phase according to embodiments.

FIG. 7a represents a flowchart of autonomous operational mode according to embodiments.

FIG. 7b represents a flowchart of sensor data processing according to embodiments.

FIG. 7c represents a flowchart of sensor data acquisition by sleep health opportunity monitors according to embodiments.

FIG. 7d represents a flowchart of trigger analysis by sleep health opportunity monitors according to embodiments.

FIG. 7e represents a flowchart of therapy analysis by protocol management system and generation of therapy modification instructions according to embodiments.

FIG. 7f represents a flowchart of therapy delivery by oral therapy device according to embodiments.

FIG. 7g represents a flowchart of distributed mode according to embodiments.

FIG. 7h represents a flowchart of sensor data processing according to embodiments.

FIG. 7i represents a flowchart of sensor data acquisition by sleep health opportunity monitors according to embodiments.

FIG. 7j represents a flowchart of trigger analysis by sleep health opportunity monitors according to embodiments.

FIG. 7k represents a flowchart of therapy analysis by protocol management system and generation of therapy modification instructions according to embodiments.

FIG. 7l represents a flowchart of transmission of therapy instructions to a collaborative device and to an oral therapy device according to embodiments.

FIG. 7m represents a flowchart of therapy delivery by oral therapy device according to embodiments.

FIG. 7n represents an embodiment of transmission of therapy activation request by a remote device.

FIG. 8a represents a flowchart of data exchange mode according to embodiments.

FIG. 8b represents a flowchart of data transmission from individual user oral therapy device to OASIS cloud system according to embodiments.

FIG. 8c represents a flowchart of data transmission of individual user to anonymous user database within OASIS cloud according to embodiments.

FIG. 8d represents a flowchart of data transmission of multiple users to anonymous user database within OASIS cloud according to embodiments.

FIG. 8e represents a flowchart of anonymous multiple user data to Heuristic Protocol Development Module within OASIS cloud according to embodiments.

FIG. 8f represents a flowchart of identification of multiple general population profiles within OASIS cloud according to embodiments.

FIG. 8g represents a flowchart of revision of user-specific protocol management system within OASIS cloud according to embodiments

FIG. 8h represents a flowchart of transmission of revised user-specific protocol management system to user base station microprocessor according to embodiments.

FIG. 8i represents a flowchart of updating of protocol management system within a user oral therapy device according to embodiments.

FIG. 9a represents a cross sectional view of a user anatomy depicting normal mandible position and normal temporomandibular joint orientation according to embodiments.

FIG. 9b represents a cross sectional view of a user anatomy depicting an advanced mandibular position and resulting displaced temporomandibular joint orientation according to embodiments.

FIG. 9c represents a close-up cross-sectional view of temporomandibular joint anatomy according to embodiments.

FIG. 9d represents close-up cross-sectional superimposed view of normal and displaced temporomandibular joint according to embodiments.

FIG. 9e represents close-up cross-sectional superimposed view of articular disk according to embodiments.

FIG. 10a represents a side view of a base station, tethering cable and oral therapy device according to embodiments.

FIG. 10b represents a cross sectional view of base station UV light used for oral therapy device sanitation according to embodiments.

### DETAILED DESCRIPTION

In the following detailed description, reference is made to the accompanying drawings that show, by way of illustration, specific embodiments in which the methods and structures may be practiced. These embodiments are described in sufficient detail to enable those skilled in the art to practice the embodiments. It is to be understood that the various embodiments, although different, are not necessarily mutually exclusive. For example, a particular feature, structure, or characteristic described herein, in connection with one embodiment, may be implemented within other embodiments without departing from the spirit and scope of the embodiments. In addition, it is to be understood that the location or arrangement of individual elements within each disclosed embodiment may be modified without departing from the spirit and scope of the embodiments.

The following detailed description is, therefore, not to be taken in a limiting sense, and the scope of the embodiments is defined only by the appended claims, appropriately interpreted, along with the full range of equivalents to which the claims are entitled. In the drawings, like numerals may refer to the same or similar functionality throughout the several views. The terms “over”, “to”, “between” and “on” as used herein may refer to a relative position of one layer with respect to other layers. One structure and/or a portion of a structure “over” or “on” another portion or bonded “to” another portion may be directly in contact with the other structures/portions or may have one or more intervening structures. Structures “adjacent” to one another may or may not have intervening structures/layers between them.

Various implementations of the embodiments herein may be formed or carried out within an oral cavity of a human user. A user, which typically comprises a human user of the embodiments herein, may be described as a sleep apnea patient, people who suffer medical problems associated with sleep disruption or sleep-disordered breathing (SBD), e.g. snoring, sleep apnea, people undergoing sleep studies, temporomandibular joint discomfort patients etc. The embodiments herein include methods and structures for the fabrication of an intelligent micro-adaptive sleep system apparatus for reducing deleterious effects of sleep apnea and other sleep-related disorders, while increasing user utilization of such an apparatus. Those methods/structures may include providing an oral therapy device comprising a top member, wherein the top member fits over at least a portion of the upper teeth within the oral cavity of a user, and wherein the top member comprises at least one embedded sensor, a port for a tethering cable, a battery, and a microprocessor with digital storage and proprietary operating system. A bottom member, which may fit over at least a portion of the bottom teeth within the oral cavity of the user, may further comprise an electrical and/or mechanical stimulus apparatus, mandibular positioning apparatus, one or more embedded sensors, and may be coupled by a hinge to the top member.

The oral therapy device may be capable of delivering various forms of therapy as directed by an intelligent Protocol Management System (“PMS”) utilizing both individual user and aggregate population data analysis.

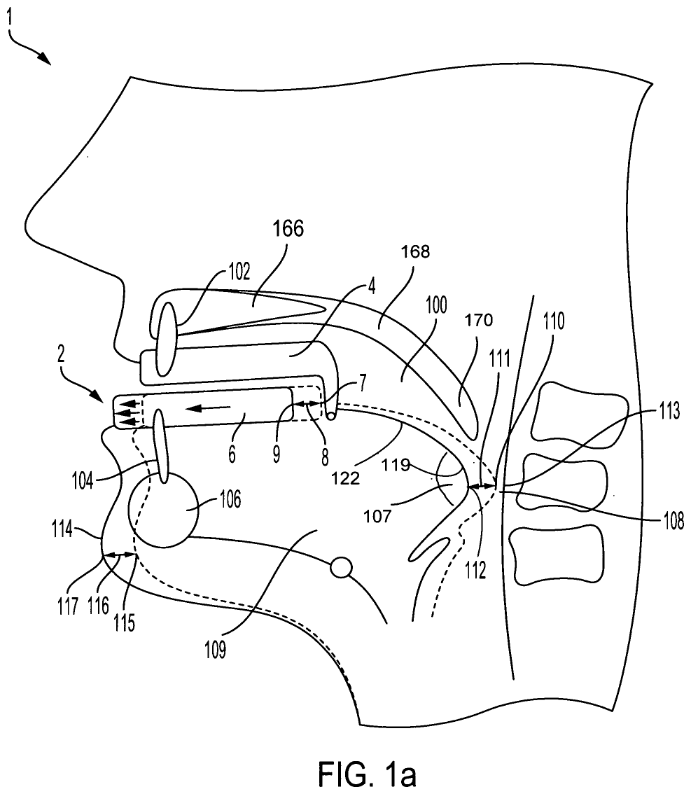

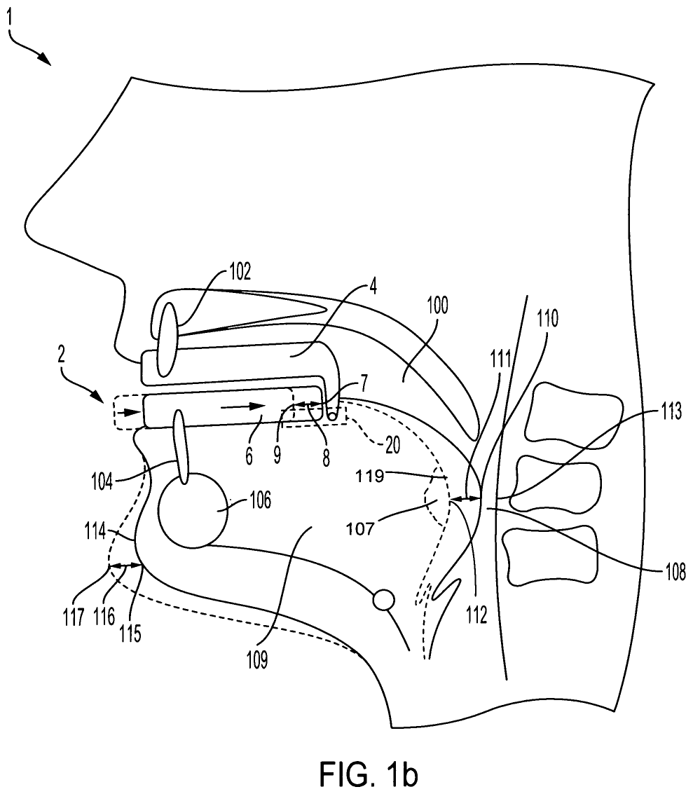

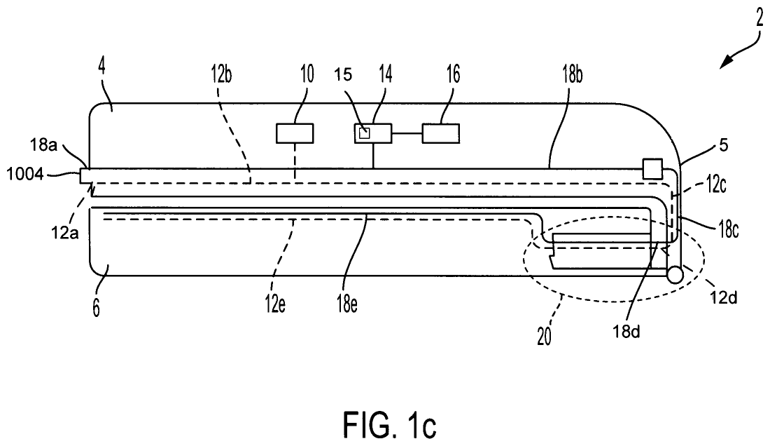

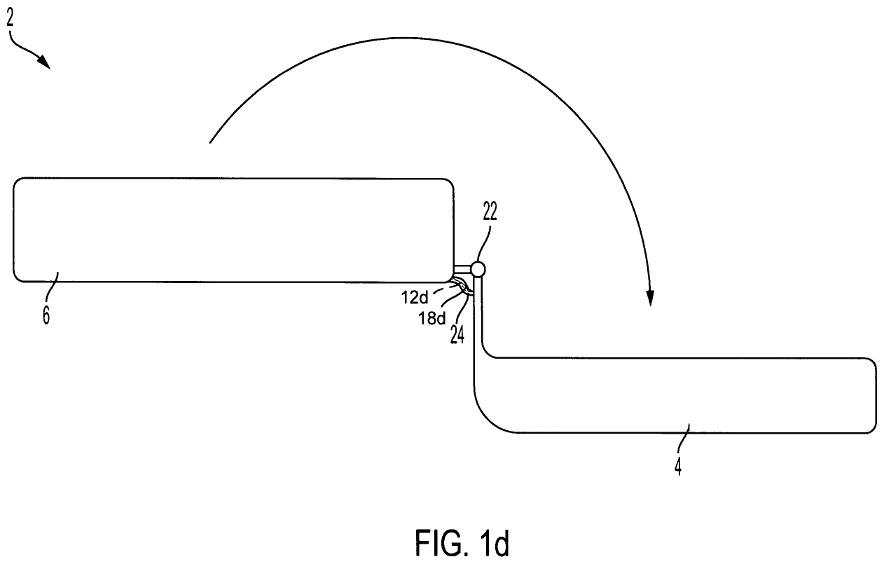

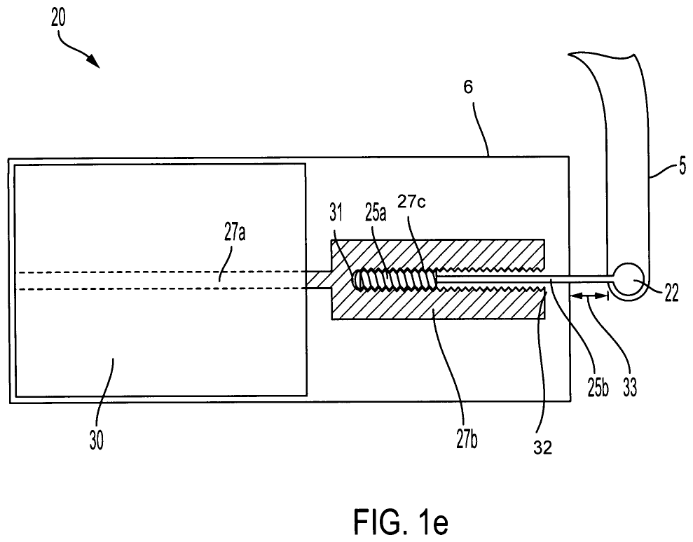

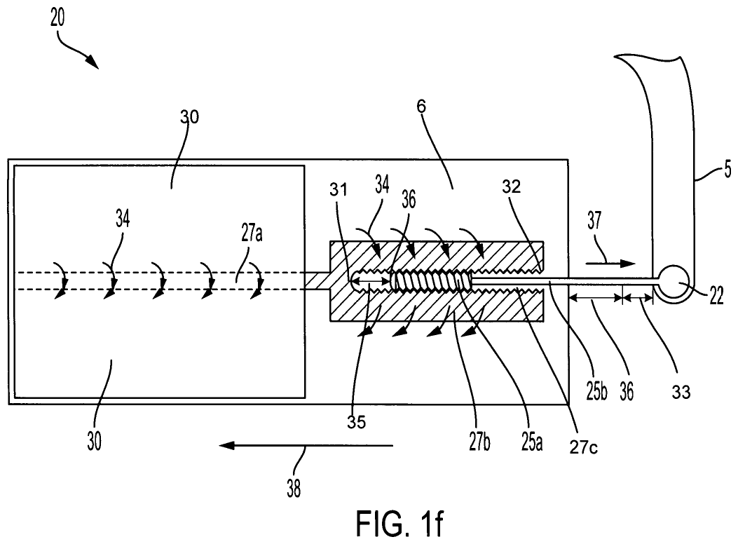

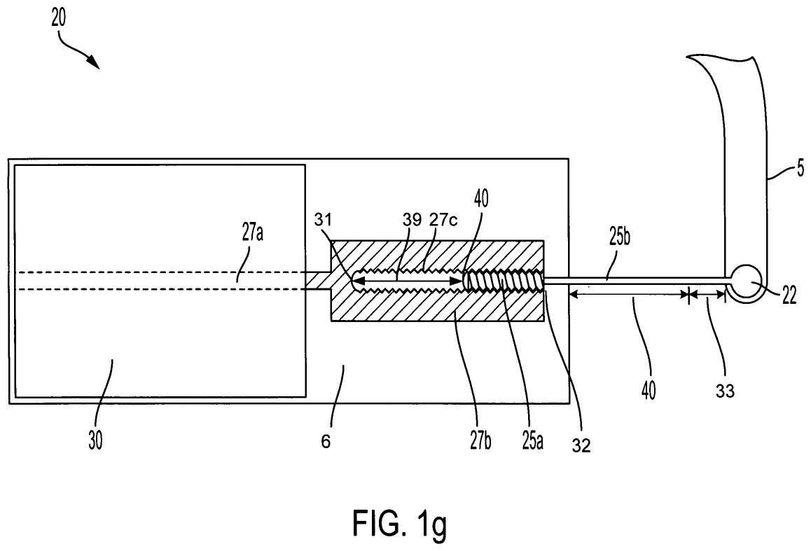

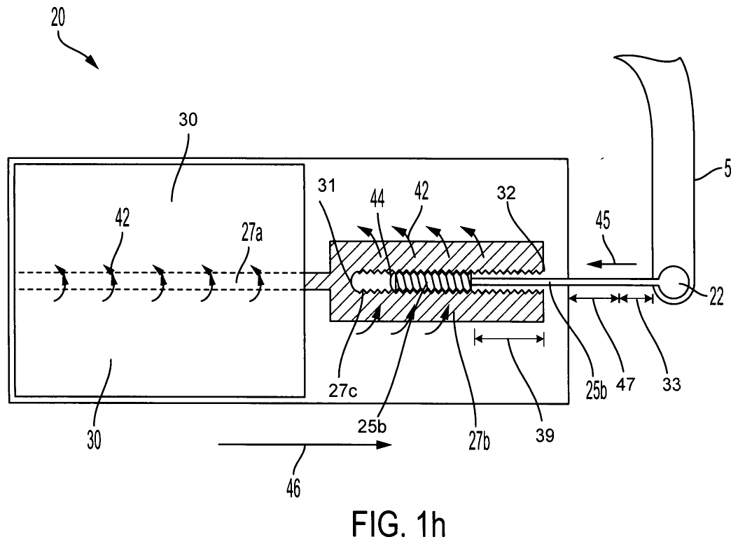

FIG. 1.a. is a sagittal view of an embodiment of the mid-face and neck area of a user 1 wherein an oral therapy device 2, has been positioned within the oral cavity 100 of the user 1. The oral therapy device 2 comprises a top member 4 and a bottom member 6. The top member 4 and bottom member 6 are comprised of a rigid substrate covered by a plastic material; however other materials may be utilized in the construction, such as but not limited to a resilient and flexible material sufficient to accommodate desired operation. Both the top member 4 and bottom member 6 may be created from a mouth mold and may be self-fitted by the user 1, reducing the need for physician or dentist visit. The top member 4 and bottom member 6 are formed around a rigid framework which is sealed to protect and isolate interior components. The top member 4 and the bottom member 6 are physically coupled to one another.

The top member 4 is at least partially in contact with the upper teeth 102 and the bottom member is at least partially in contact with the lower teeth 104 of the user 1. The oral therapy device 2 is capable of providing micro-adaptive hands-free therapeutic positioning of a user's mandible 106. The bottom member 6 of the oral therapy device 2 is capable of moving forward (or backward) in response to signals received from an integral microprocessor (see FIG. 1c) located within the oral therapy device 2.

The passageway through which humans breathe sometimes comprises a small opening that is bordered by numerous anatomical structures. An oral cavity 100, may be adjacent to a hard palate 166, a soft palate 168, a uvula 170 and a top surface of a tongue 122. A portion of an oropharynx 108 may be bordered by a posterior pharynx 113, a portion 107 of a base of a tongue 109, the soft palate 168 and the uvula 170.

Obstruction of the passageway through which humans breathe usually occurs when one or more anatomical structures contact each other, reducing or completely closing off the cross-sectional area of the oropharynx through which air may pass. A reason for this may be that during sleep, muscle tension lessens and the portion 107 of the base of the tongue 109 may fall backwards and the surface 119 of the portion 107 of the base of the tongue 109 may contact the posterior pharynx 113 and/or the soft palate 168 and/or the uvula 170. In most humans, the passageway becomes obstructed during sleep in the area of the portion of the oropharynx 108 adjacent to the portion 107 of the base of the tongue 109 and the posterior pharynx 113.

In an embodiment, the bottom member 6 is capable of advancing the user's 1 mandible 106 and the mentum of the chin 114 to a more-forward position by the use of a mandibular positioning drive (MPD to be further described in FIGS. 1e-1h) under the direction of an integral microprocessor (see FIG. 1c). The oral therapy device 2 is capable of providing a therapeutic protocol, which provides a mechanism to increase a cross-sectional area of the portion 108 of the oropharynx. The portion of the oropharynx 108 herein comprises the region directly adjacent to the user's 1 posterior pharynx 113 and the portion 107 of the base of the tongue 109. The therapeutic protocol provided by the oral therapy device 2 mitigates sleep-related breathing disorders by increasing the cross-sectional area through which air may be freely drawn into the user's 1 lungs. Forward movement of the bottom member 6, effected by the MPD as directed by the microprocessor (see FIG. 1c), may advance the user's 1 mandible 106 and thus may pull the surface 119 of the portion 107 of the base of the tongue 109 away from the posterior pharynx 113 of the user 1. In this manner, the cross-sectional area of the portion of the oropharynx 108 is increased.

In an embodiment, during active therapy (wherein the bottom member is advanced from a baseline position) the bottom member 6 may be moved from a first position 7 (shown as a dotted line) to a second position 9, thereby effecting mandibular advancement. In an embodiment, there is a distance 8 between the first position 7 and the second position 9 of the bottom member 6. In an embodiment, the distance may be between about 1 millimeter to 1 centimeter. In other embodiments, the distance range may vary according to the particular physiology of a particular user. In addition, during active therapy the surface 119 of the portion 107 of the base of the tongue 109 may be advanced from a first position 110 (shown as a dotted line) to a second position 112. Also, during active therapy, the mentum of the chin 114 may be advanced from a first position 115 (shown as a dotted line) to a second position 117.

A distance 116 between the first position 115 and the second position 117 of the mentum of the chin 114 may comprise approximately the same magnitude as a magnitude of the distance 8 that is between the first position 7 and the second position 9 of the bottom member 6. In an embodiment, a distance 111 between the first position 110 and the second position 112 of the surface 119 of the portion 107 of the base of the tongue 109 may comprise a smaller magnitude than the distance 8 between the first and second positions 7,9 of the bottom member 6, and/or may comprise a smaller magnitude than the distance 116 between the first and second positions 115,117 of the mentum of the chin 114. In an embodiment, the smaller magnitude of the distance 111 may be due to the elasticity of the base of the tongue 109 and the portion 107 of the base of the tongue 109 and other surrounding anatomical structures. The distance 111 by which the surface 119 of the portion 107 of the base of the tongue 109 may be pulled away from the posterior pharynx 113 during active therapy may vary from its first (pre-therapy) position 110 among various users 1, based upon anatomical differences.

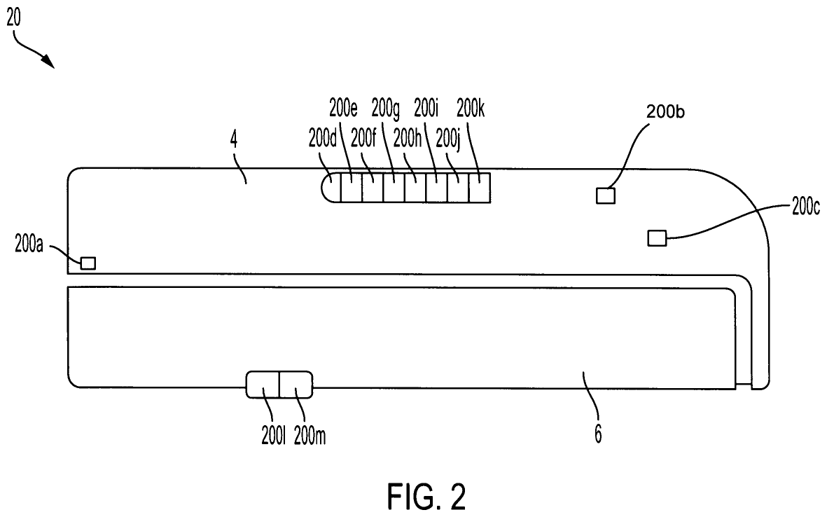

Active therapy may be initiated by the microprocessor (shown in FIG. 1c) as determined by data received by the microprocessor from sensors (shown in FIG. 2) which may be located within the oral therapy device 2, as will be described subsequently herein. The distance by which the bottom member 6 is advanced is variable and heuristically controlled by the microprocessor within the oral therapy device 2 to enhance user 1 sleep quality based upon analysis of various sensory data parameters. In some cases, mandibular 106 advancement effected by the oral therapy device 2 results in a Class 3 occlusion with an underbite position of the upper 102 and lower teeth 104.

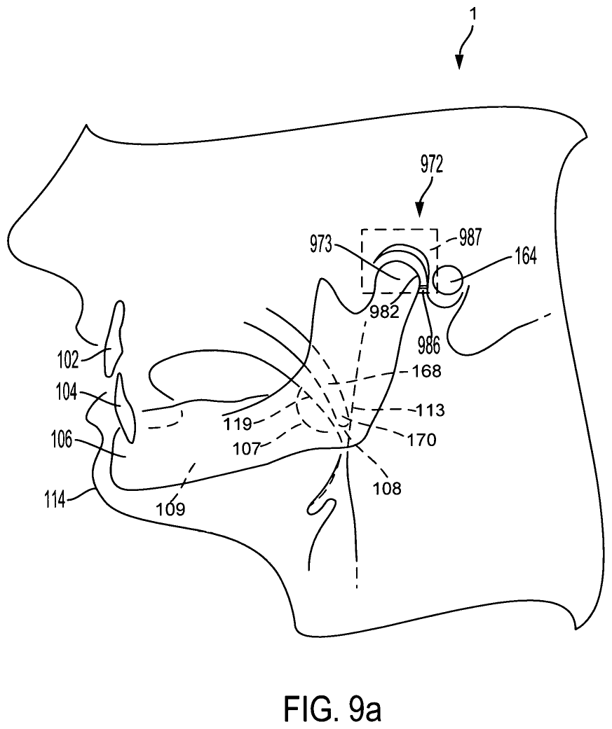

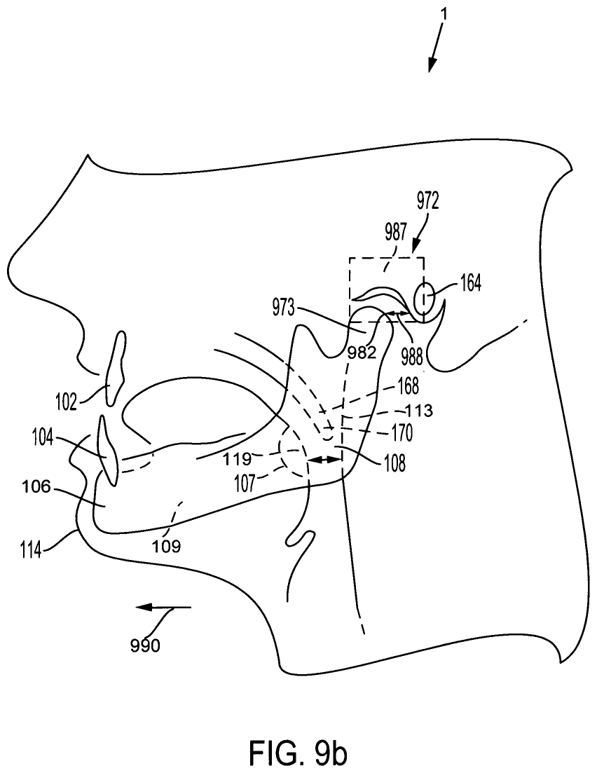

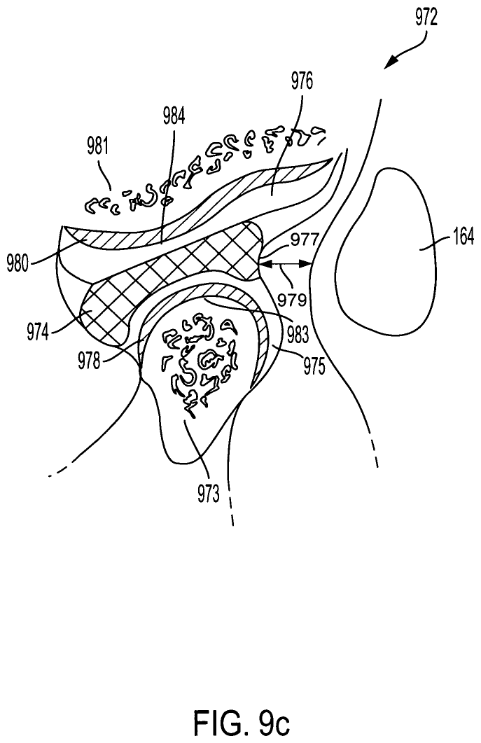

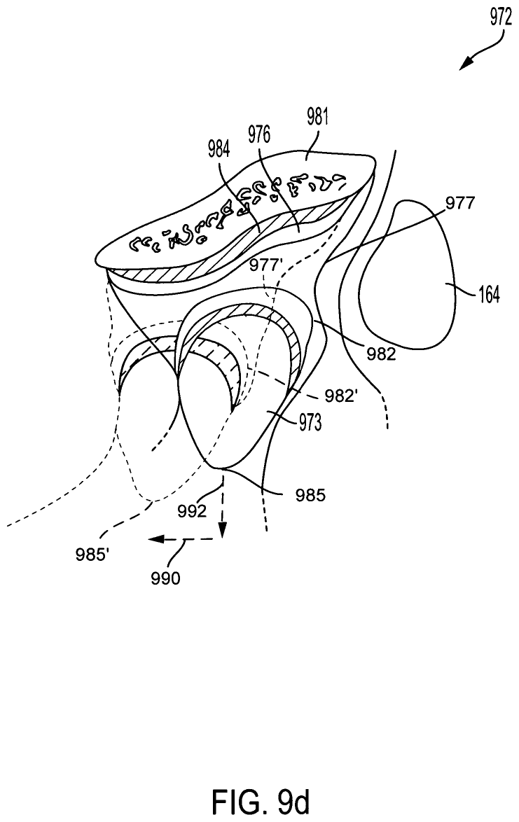

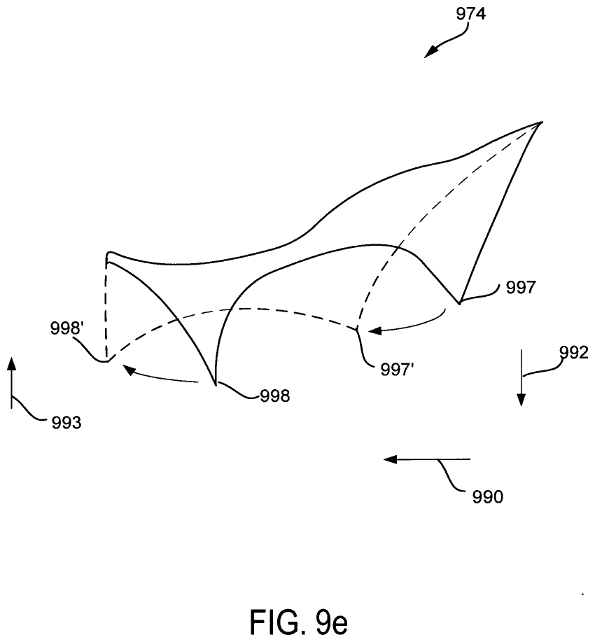

FIG. 1.b. depicts an embodiment of the retraction of the mandible 106 (as opposed to mandibular advancement as shown in FIG. 1.a). The bottom member 6 is capable of retracting the user's 1 mandible 106 and the mentum of the chin 114 from the second position 117 (shown as a dotted line) as previously depicted in FIG. 1a to a position closer to the first position 115. The retraction may be achieved by the use of a mandibular positioning drive (MPD to be further described in FIGS. 1e-1f). In an embodiment, the microprocessor (see FIG. 1c) controls retraction of the mandible 106, wherein the microprocessor receives sensory data, wherein the microprocessor analyzes the sensory data to optimize the magnitude of retraction required by a particular course of sleep disordered breathing therapy to be applied to the user 1. In another embodiment, the mandible 106 may be periodically retracted to ease strain on the temporomandibular joint (TMJ), to be discussed further in FIGS. 9a-9e. The distance retracted and the time that the mandible 106 remains in a particular position may be optimized by the microprocessor in order to alleviate TMJ strain during the application of therapy.

In an embodiment, during active therapy, the bottom member 6 may be retracted from the second position 9 (shown as a dotted line) to the first position 7 or any other distance 8 from the first position 7. In an embodiment, the second position 9 may comprise any number of distances from the first position 7. In an embodiment, the distance 8 between the second position 9 and the first position 7 of the bottom member 6 may be between about 1 millimeter to 1 centimeter. In other embodiments, the distance range may vary according to the particular physiology of a particular user. In addition, during retraction of the mandible 106 the surface 119 of the portion 107 of the base of the tongue 109 may be moved from the second position 112 (shown as a dotted line) to the first position 110. Also, during retraction, the mentum of the chin 114 may be moved from a second position 117 (shown as a dotted line) to a first position 115.

A distance 116 between the first position 115 and the second 117 position of the mentum of the chin may comprise approximately the same magnitude as a magnitude of the distance 8 that is between the first position 7 and the second position 9 of the bottom member 6. In an embodiment, a distance 111 between the first position 110 and the second position 112 of the surface 119 of the portion 107 of the base of the tongue 109 may comprise a smaller magnitude than the distance 8 between the first and second positions 7,9 of the bottom member 6, and/or may comprise a smaller magnitude than the distance 116 between the first and second positions 115,117 of the mentum of the chin 114. In an embodiment, the smaller magnitude of the distance 111 may be due to the elasticity of the base of the tongue 109 and the portion 107 of the base of the tongue 109 and other surrounding anatomical structures. The distance 111 by which the surface 119 of the portion 107 of the base of the tongue 109 may be pulled away from the posterior pharynx 113 during active therapy may vary from its first (pre-therapy) position 110 among various users 1, based upon anatomical differences.

The distance by which the bottom member 6 is retracted is variable and intelligently controlled by the microprocessor (see FIG. 1c) located within the oral therapy device 2 to enhance user sleep quality based upon analysis of various sensory data parameters. Retraction of the mandible 106 and the mentum of the chin 114 to a less advanced position by the oral therapy device 2 may be determined by several potential factors including but not limited to, sensory data analysis and/or to provide a periodic easing of stress to the user's 1 TMJ (as described further in FIGS. 9a-9e).

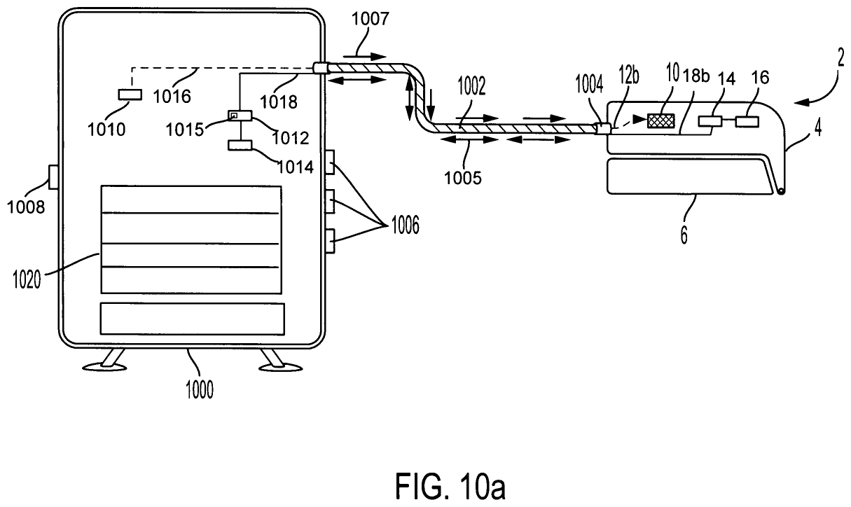

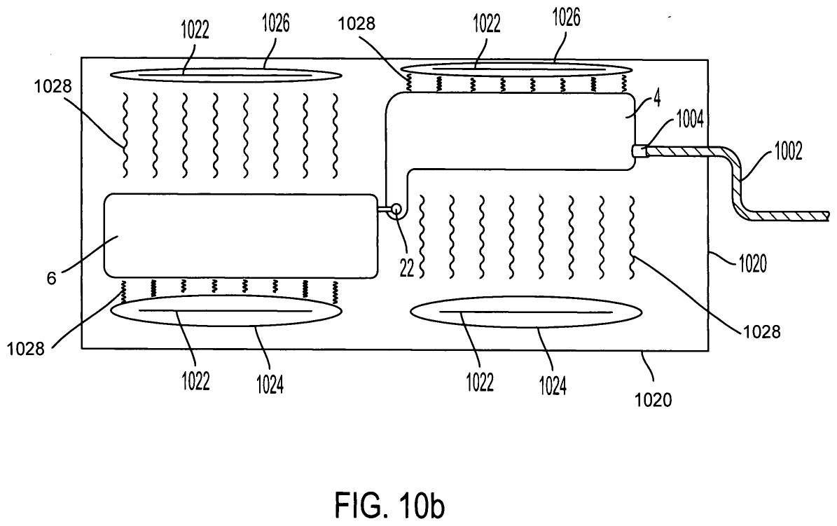

FIG. 1.c. depicts an embodiment of a cross-sectional view of the oral therapy device 2. In an embodiment, various components of the oral therapy device 2 may be removed and/or re-used in a second, or replacement oral therapy device 2 framework, if required, to accommodate repositioning of the user's 1 bite profile, for example. The oral therapy device 2 is powered by electrical current provided via a small battery 10 capable of providing sufficient power to operate all electrical components during autonomous and wireless mode operations. In an embodiment, the battery 10 may be located within the top member 4, however, in other embodiments, the battery 10 may be located within the bottom member 6. In an embodiment, the battery 10 may be a rechargeable battery, and may be recharged via a base station (to be described subsequently further in herein and depicted in FIGS. 10a-10b).

Electrical power is delivered to various sensors 200a-200m and multiple therapy apparatus actuators, such as a mandibular positioning drive (MPD) actuator (to be described subsequently herein), for example, by an electrical bus 12 (shown as a dotted line). In an embodiment, the electrical bus 12 may comprise a first portion 12a that is located within a portion of a tethering port 1004 (to be described subsequently herein) that extends outward from the top member 4, a second portion 12b that extends through the top member 4, a third portion 12c that extends through the vertical portion 5 of the top member 4, a fourth portion 12d that extends through an umbilical region (to be described further in FIG. 1d), and a fifth portion 12e that extends through the bottom member 6 of the oral therapy device 2. The electrical bus 12 is physically and electrically coupled to the battery 10, a microprocessor 14, storage memory 16 and a data bus 18.

Data collected from various sensors 200a-200m, which reflect the user's sleep status from a user 1, may be retained by the storage memory 16. In an embodiment, the storage memory 16 and the microprocessor 14 may be located within the top member 4, and may be adjacent to one another. The microprocessor 14 may include a communicative device 15, such as an antenna or any other suitable structure/device to enable sending and receiving data when operating in either a wireless mode or when connected to a base station via a tether (see FIG. 10a). In other embodiments, the storage memory 16 and microprocessor 14 may be located within the bottom member 6. The data bus 18 may transfer data between a microprocessor 14, the sensors 200a-200m and the actuators (to be described subsequently herein). In an embodiment, the data bus 18 may comprise a first portion 18a that is located within a portion of a tethering port 1004 that extends outward from the top member 4, a second portion 18b that extends through the top member 4, a third portion 18c that extends through the vertical portion 5 of the top member 4, a fourth portion 18d that extends through an umbilical region (to be described further in FIG. 1d), and a fifth portion 18e that extends through the bottom member 6 of the oral therapy device 2. A mandibular positioning drive system 20 may be located within the bottom member 6 and may be coupled to the electrical bus 12.

FIG. 1.d. depicts an embodiment of the manner in which the top member 4 may be affixed to the bottom member 6 at an attachment point 22 where the top member 4 and the bottom member 6 are attached to one another. In an embodiment, the attachment point 22 may function as a hinge, and may allow the top member 4 to open between a 0-degree angle to about a 180-degree angle relative to the bottom member 6. A flexible umbilical-type connector 24 may be between the top member 4 and bottom member 6, adjacent to the attachment point 22, through which a portion of the electrical bus 12d and a portion of the data bus 18d may reside and may enable power transfer and data share between the top member 4 and bottom member 6. The attachment point 22 is comprised of a metallic substance, however other materials may be used in its construction. The attachment point 22 is located such that the user 1 feels no or minimal discomfort.

FIG. 1e. depicts an embodiment of a close-up cross-sectional view of the mandibular positioning drive 20 that may be located within the bottom member 6. The embodiments described herein may take other forms including but not limited to hydraulic piston-type mechanism, air pressure piston-type mechanism, solenoid valves, piezoelectric motor usage, etc. according to particular design requirements. In an embodiment, the mandibular positioning drive 20 comprises a first portion of a sleeve 27 capable of rotation in both clockwise and counter-clockwise directions. The first portion of the sleeve 27a is in physical contact with one or more electric motors 30. The second portion of the sleeve 27b is hollow and threaded on the inner surface of the sleeve 27c. The mandibular positioning drive 20 further comprises a first portion of a shaft 25a that is threaded and in contact with the inner surface 27c of the second portion of the sleeve 27b. A second portion of the shaft 25b extends outward from the first portion of the shaft 25a. The second portion of the shaft 25b contacts the vertical portion of the top member 5 at the attachment point 22. The shaft 25a-b is incapable of rotation in any direction.

The first portion of the shaft 25a is capable of forward movement until it reaches its maximum advancement position 32 when it is in physical contact with the outer end of the inner surface 27c of the second portion of the sleeve 27b. The first portion of the shaft 25a is also capable of backward movement until it is in physical contact with the inner end 31 (the baseline position) of the inner surface 27c of the second portion of the sleeve 27b.

There is a minimum distance 33 between the vertical portion of the top member 5 and the bottom member 6 that represents a baseline (zero mandibular advancement) position of both the top member 4 and the bottom member 6. This distance 33 may be between 5 to 50 millimeters.

FIG. 1f. depicts the provision of mandibular advancement therapy by the oral therapy device 2 through forward movement of the bottom 6. In an embodiment, the motor (s) 30 may cause the first portion of the sleeve 27a to rotate in a clockwise direction 34. The clockwise rotation direction 34 of the second portion of the sleeve 27b causes the shaft 25a-b to move outward in a direction 37 from the end 31 of the threaded portion 27c of the second portion of the sleeve 27b. Movement of the shaft portions 25a, 25b in the direction 37 causes the bottom member 6 to move away from the vertical portion of the top member 5 in a direction 38, in an embodiment.

A distance 35 by which the portions of the shaft 25a,25b have moved away from the end 31 of the threaded portion 27c of the second portion of the sleeve 27b may be approximately 0.5 centimeters in an embodiment. A distance 36 by which the bottom member 6 has moved away from the minimum distance 33 between the bottom member 6 and the vertical portion of the top member 5 may be approximately 0.5 centimeters, in an embodiment. Once a desired mandibular 106 position is achieved by the oral therapy device 2, contact between the threaded inner surface 27c of the second portion of the sleeve 27b and the first portion of the shaft 25a acts as a brake within the mandibular positioning drive 20 to allow such position to be maintained without continuous motive force by the electric motor (s) 30.

FIG. 1g. depicts mandibular advancement therapy provided by the oral therapy device 2 through forward movement of the bottom member 6. In an embodiment, the motor (s) 30 have effected rotation of the first portion of sleeve 27a and the second portion of the sleeve 27b causing the shaft portions 25a,25b to move outward until it they have reached their maximum advancement position 32 whereby the shaft 25a is in physical contact the outer end of the inner surface 27c of the second portion of the sleeve 27b.

A distance 39 by which the shaft portions 25a, 5b have moved away from the inner end 31 of the threaded portion 27c of the second portion of the sleeve 27b may be approximately 1.0 centimeters, in an embodiment. The distance 40 by which the bottom member 6 has moved away from the minimum distance 33 between the bottom member 6 and the vertical portion of the top member 5 may be approximately 1.0 centimeters, in an embodiment.

FIG. 1.h. depicts mandibular retraction therapy by the oral therapy device 2 through backward movement of the bottom 6, according to an embodiment. In an embodiment, the motor (s) 30 are causing the first portion of the sleeve 27a to rotate in a counter-clockwise direction 42. A Corresponding counter-clockwise rotation direction 42 of the second portion of the sleeve 27b causes the shaft portions 25a,25b to move inward in a direction 45 from the maximum advancement position 32 toward the inner end 31 of the threaded portion 27c of the second portion of the sleeve 27b. Movement of the shaft portions 25a,25b in the direction 45 causes the bottom member 6 to move toward the vertical portion of the top member 5 in the direction 46. The distance 39 by which the shaft portions 25a,25b have been reduced from the maximum advancement position 32 of the second portion of the sleeve 27b may be between approximately 0.5 centimeters, in an embodiment. A distance 47 by which the bottom member 6 has now approached the minimum distance 33 between the vertical portion of the top member 5 and the bottom member 6 may be approximately 0.5 centimeters, in an embodiment.

In an embodiment, the distance by which the bottom member 6 is retracted is variably and intelligently controlled by the microprocessor 14 located within the oral therapy device 2 to enhance user sleep quality based upon analysis of various sensory data parameters. Retraction of the bottom member 6 of oral therapy device 2 may be determined by several potential factors including but not limited to, sensory data analysis and/or to provide a periodic easing of stress to the user's 1 TMJ (to be described more fully in FIGS. 9a-9e).

FIG. 2 depicts a cross sectional view of one or more internal sensors 200a-200m that may be physically embedded within at least one of the top member 4 or the bottom member 6 of the oral therapy device 2, according to an embodiment. Data collected by the one or more sensors 200a-200m may have an impact upon sleep state and sleep quality of the user 1. In an embodiment, the top member 4 may have such sensors as an alignment sensor 200a, wherein the alignment sensor is capable of verifying the proper initial alignment of the top member 4 relative to the bottom member 6 of the oral therapy device 2.

In other embodiments, the top member 4 may include an auditory sensor 200b that is capable of measuring the amount of snoring of the user 1, an airflow sensor 200c that is capable of measuring airflow volume through the top member 4, a head orientation sensor 200d that is capable of determining the user's 1 head position, a pH sensor 200e that is capable of measuring oral chemistry of the user 1 and any number of additional sensors capable of measuring related health aspects, e.g., a H. pylori detection sensor 200f, dry mouth sensor 200g, acid reflux detection sensor 200h, cardiovascular operation sensor 200i, TMJ stress sensor 200j, body position sensor 200k and any other potentially health-related conditions. Although FIG. 2 depicts the sensors 200a-200k as being physically located within the top member and also being adjacent to one another, the sensors 200a-200k may be located at any desired location within the top member 4, and in some embodiments may be located in any desired location within the bottom member 6, according to particular design requirements.

In an embodiment, the bottom member 6 may include an O2 sensor 200l, that is capable of measuring blood oxygen concentration of the user 1. In other embodiments, the bottom member 6 may include a body temperature sensor 200m that is capable of measuring body temperature of the user 1.

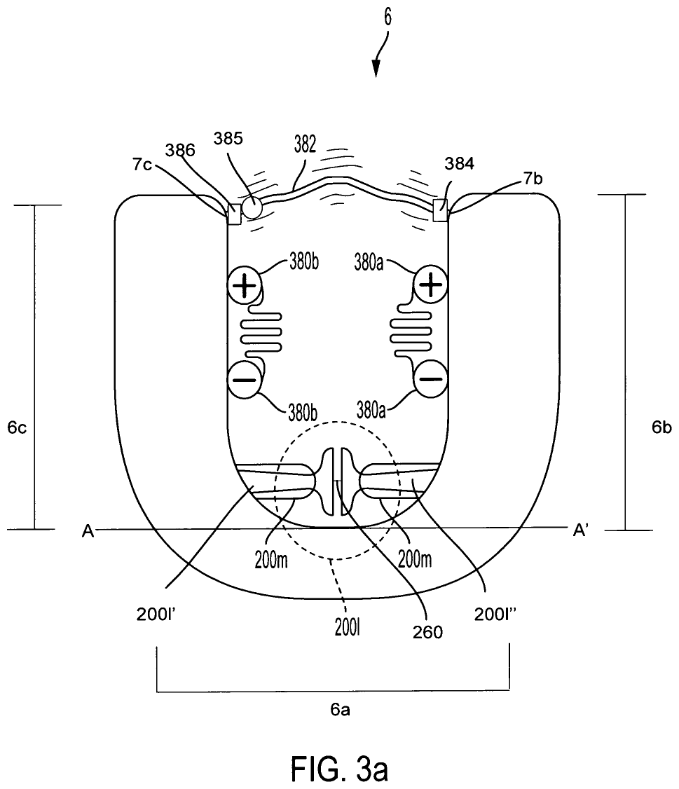

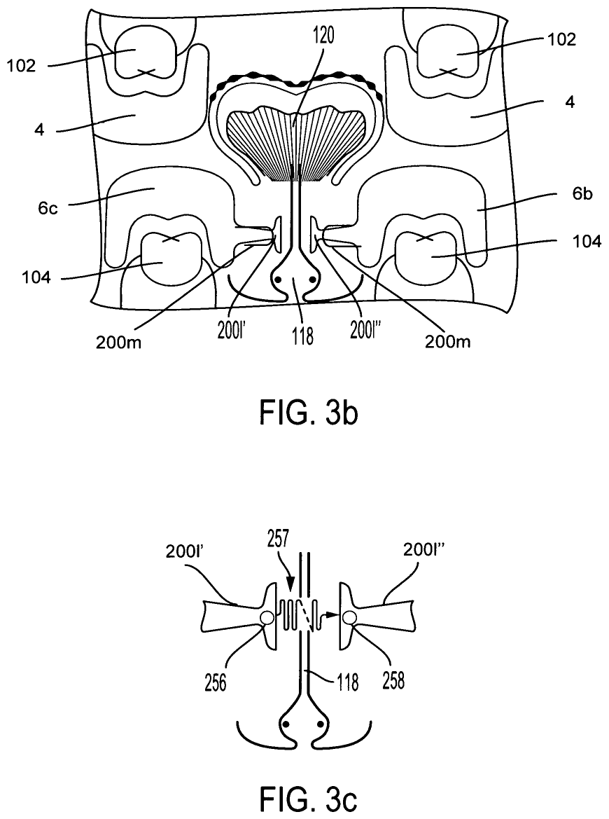

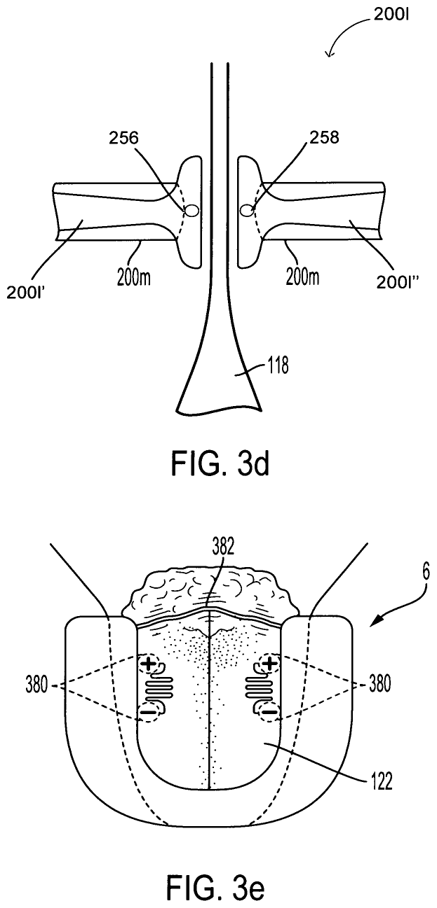

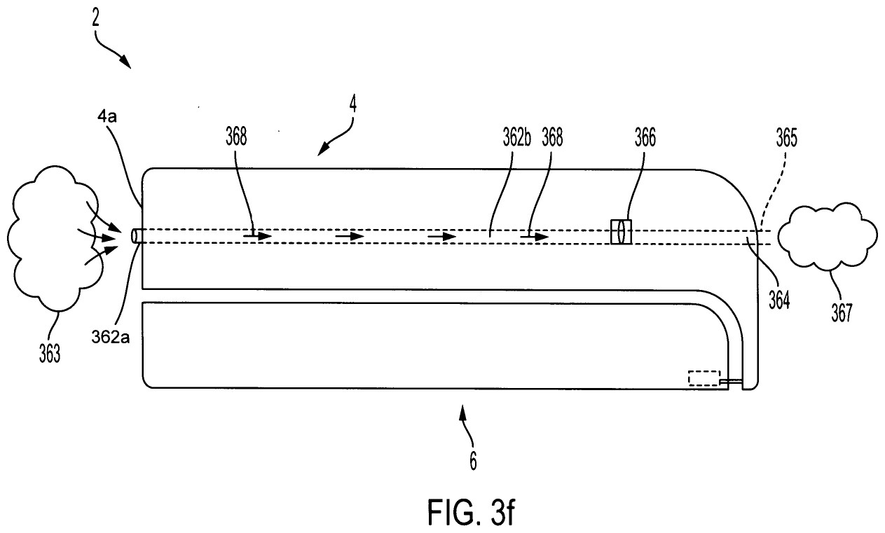

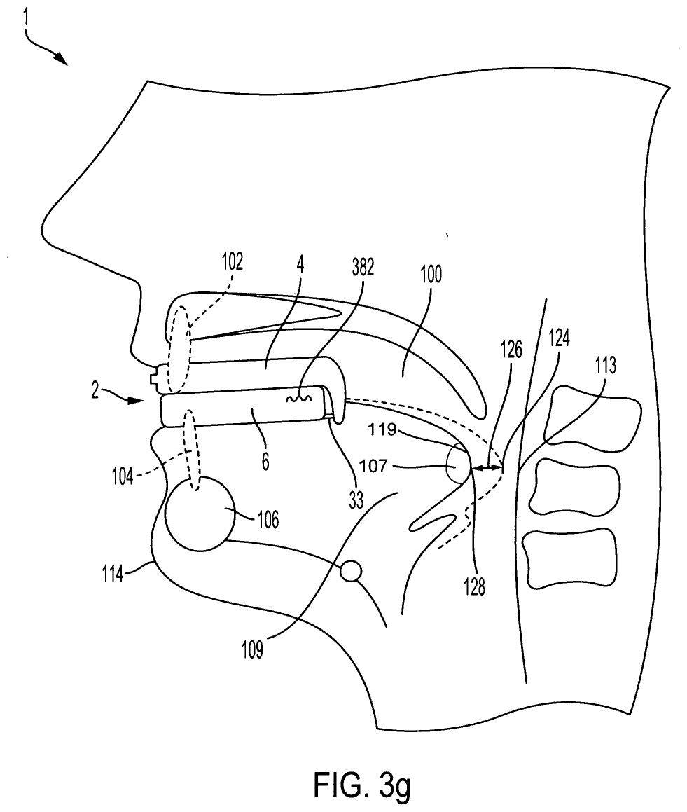

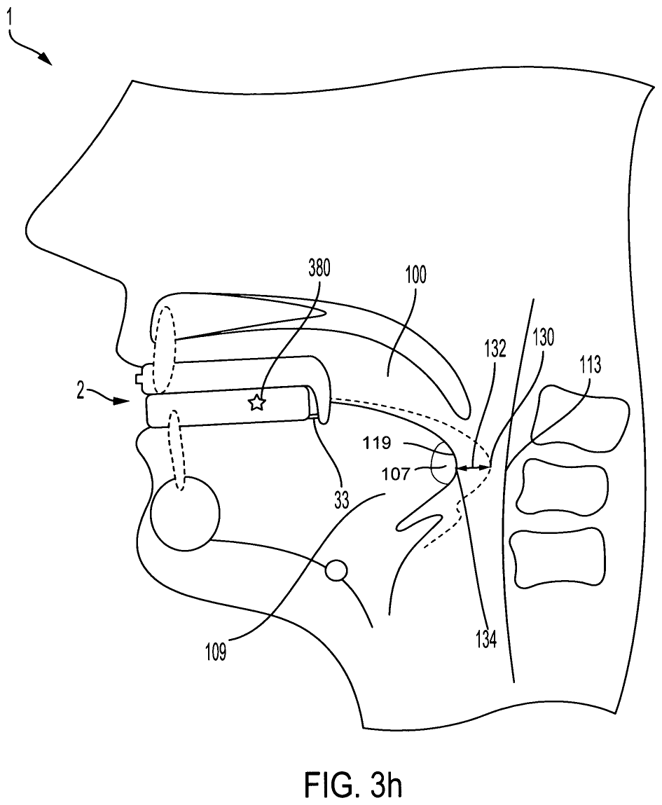

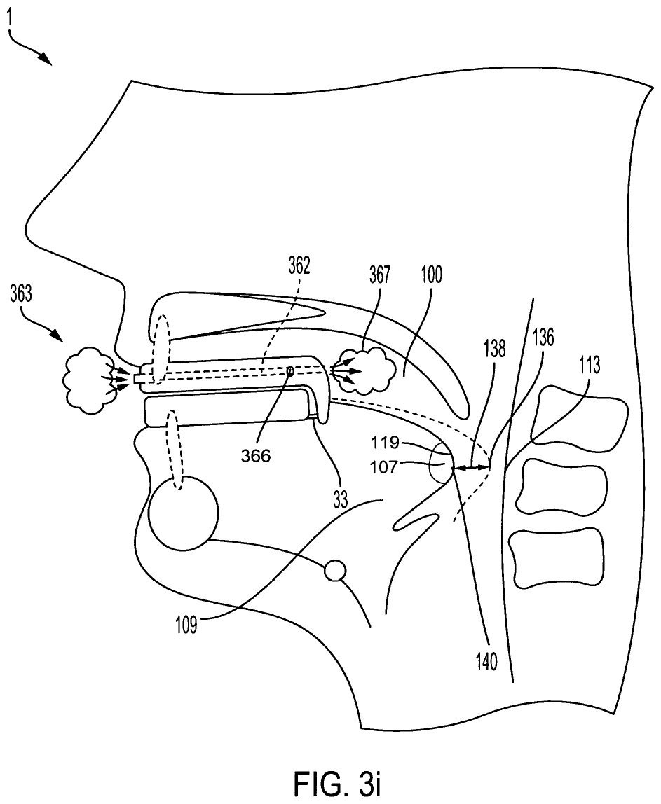

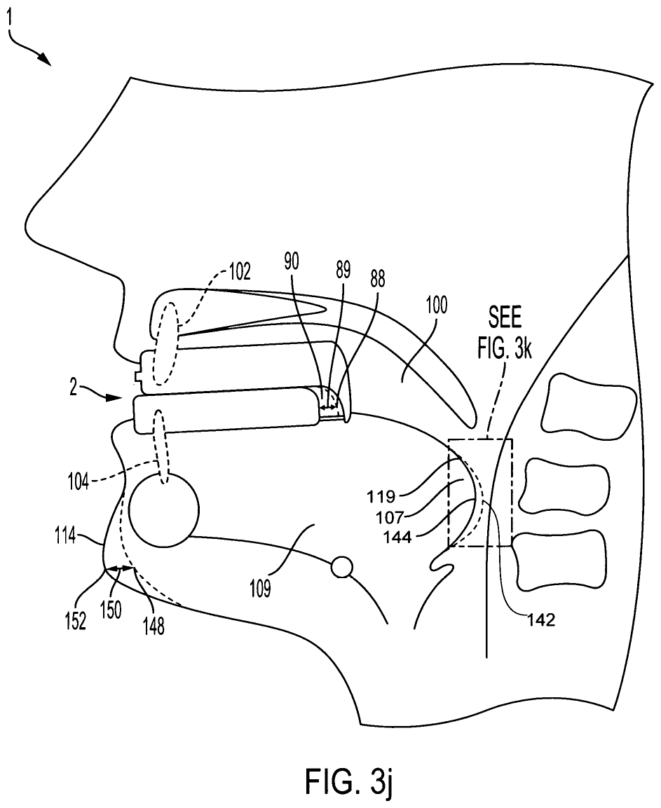

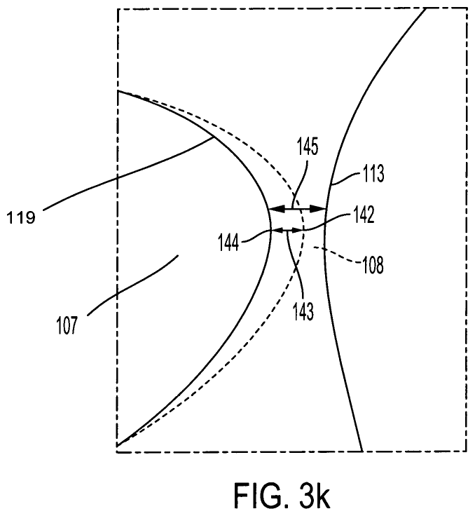

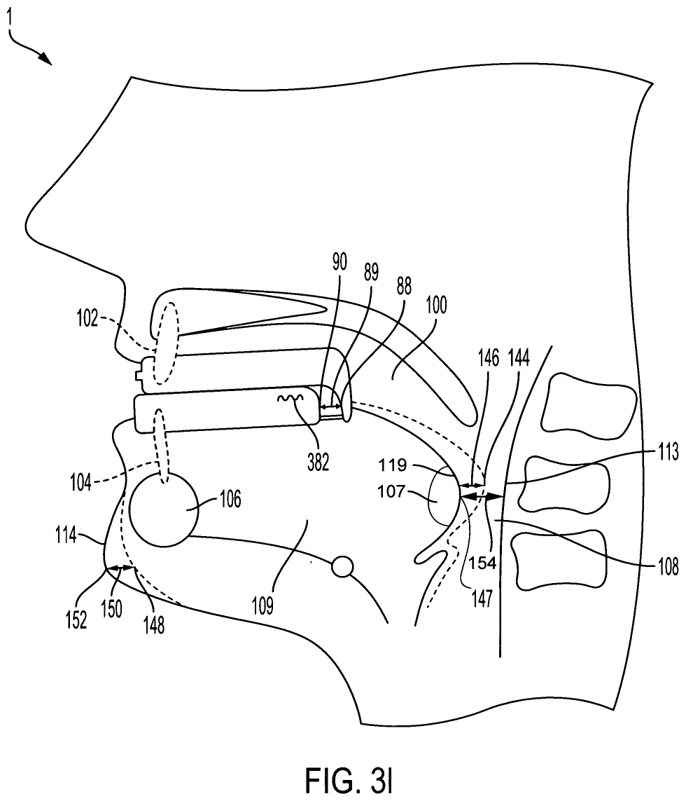

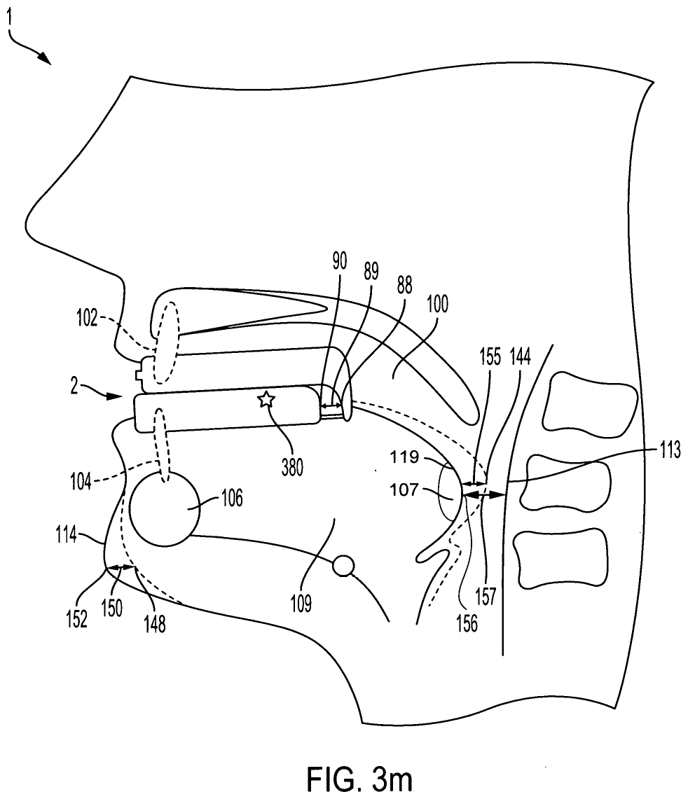

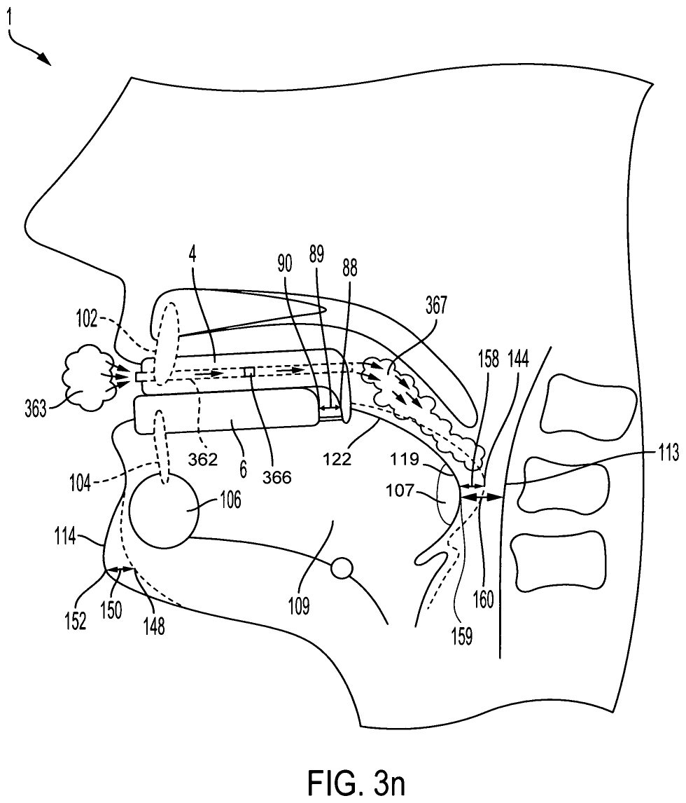

FIG. 3a is an embodiment of a top view of the bottom member 6 which comprises a first portion 6a, a second portion 6b and a third portion 6c. In an embodiment, the first section 200l′ of the O2 sensor 200l is on a surface of the third portion 6c of the bottom member 6, and a second section 200l″ of the O2 sensor 200l is on a surface of the second portion 6b of the bottom member 6. There is a gap 260 between the first section 200l′ and the second section 200l″ of the O2 sensor 200l, wherein the lingual frenulum 118 (to be shown later) may be located within the gap 260. A light beam (to be shown later) is capable of passing from the first section 200l′ through the lingual frenulum 118 to the second section 200l″. In an embodiment, the first section 200l′ may be a light emitter and the second section 200l″ may be a light detector or vice-versa depending upon particular design requirements.

In an embodiment, the first section 200l′ may comprise a length 0.25-1.5 centimeters and may comprise a linear portion with a terminal end that extends in a perpendicular direction from the linear portion of the first section 200l′. In some embodiments, a temperature sensor 200m may be in the form of a sleeve fitting over the linear portion of the first section 200l′, the second section 200l″ or both sections 200l′ and 200l″ depending upon particular design requirements. In an embodiment, while the bottom member 6 may comprise a material such as a rigid plastic material, the temperature sensor 200m may comprise a more flexible material such as rubberized plastic. In an embodiment, the gap 260 may comprise a distance wherein the distance is between about 10 and 50 millimeters. In an embodiment, the terminal ends of the first section and the second section of the sensor 200m are located adjacent to the first section 6a of the bottom member 6.

In an embodiment, a vibrational band 382 is affixed to a terminal end 7b of the second section 6b of the bottom member 6 and to a terminal end 7c of the third section 6c of the bottom member 6. In an embodiment, the vibrational band 382 has a length of between 4-6 centimeters that spans across the width of the tongue and makes physical contact with the top surface of the tongue 122. The vibrational band 382 is flexible and may comprise materials such as rubber, flexible plastic materials, flexible conductive materials, flexible piezo electric materials, etc. In an embodiment, a take-up reel 384 is affixed to the second portion 6b of the bottom member 6 and an eccentric motor 386 is affixed to the third portion 6c of the bottom member 6. One end of the vibrational band 382 is attached to the take-up reel 384 and the other end of the vibrational band 382 is attached to the eccentric motor 386.

In an embodiment, there may be a tension sensor 385 located adjacent to the vibrational band 382, which may monitor and calibrate the amount of tension in the vibrational band 382. In an embodiment, the tension sensor 385 may send tension data to the microprocessor 14. The vibrational band 382 will be positioned such that it is in physical contact with the top surface of the tongue 122. In an embodiment, the take-up reel 384 may be capable of adjusting the tension of the vibrational band 382 over the top surface of the tongue 122 and the eccentric motor 386 may be capable of causing the vibrational band 382 to vibrate and may be capable of providing tactile stimulation to the top surface of the tongue 122.

In an embodiment, the vibrational stimulation apparatus provides a mechanism for inducing the surface 119 of the portion 107 of the base of the tongue 109 from an initial therapy position close to the posterior pharynx 113 to a more forward position away from the posterior pharynx 113 increasing the area through which air may be more freely drawn into the lungs (to be further described in FIG. 3g). In an embodiment, after providing mandibular 106 advancement as a therapy protocol, it may be desired to further open the portion of the oropharynx 108 to enhance airflow mitigating sleep disruption, wherein sleep disruption may be a cause of cardiovascular stress, etc.

Adjacent to the vibrational stimulation band 382 on the bottom member 6, a first set of electrodes 380a may be at least partially embedded within a portion of the second section 6b of the bottom member 6, and a second set of electrodes 380b may be at least partially embedded within the third section 6c of the bottom member 6. In an embodiment, the first set of electrodes 380a comprises a positive terminal and a negative terminal, and the second set of electrodes 380b comprises a positive terminal and a negative terminal, wherein the first set 380a and the second set 380b are on either side of the tongue. In an embodiment, the set of electrodes 380a,380b, maintain a neutral position for the tongue between the electrodes 380a,380b.

In an embodiment, the set of electrodes 380a,380b comprise an electrical stimulation apparatus 380. In an embodiment, the electrical stimulation apparatus 380 provides a mechanism for inducing the surface 119 of the portion 107 of the base of the tongue 109 from an initial therapy position close to the posterior pharynx 113 to a more forward position away from the posterior pharynx 113 increasing the area through which air may be more freely drawn into the lungs (to be further described in FIG. 3h). In an embodiment, after providing mandibular 106 advancement as a therapy protocol, it may be desired to further open the portion of the oropharynx 108 to enhance airflow mitigating sleep disruption, wherein sleep disruption may be a cause of cardiovascular stress, etc.

FIG. 3b is an embodiment of a cross-sectional view of the second portion 6b and the third portion 6c of the bottom member 6 along the line A-A′ (as shown in FIG. 3a). The first section 200l′ and the second section 200l″ of the O2 sensor 200l are each adjacent to a side of the lingual frenulum 118, which is beneath the bottom of the tongue 120 (the tongue 120 is shown in a lifted position in FIG. 3b to better view the O2 sensor 200l beneath the tongue 120). The upper teeth 102 and the lower teeth 104 are depicted inside of the top member 4 and the bottom member 6, respectively. The lingual frenulum 118 is located in the gap 260 between the first section 200l′ and the second section 200l″ of the O2 sensor 200l.

FIG. 3c is an embodiment of a close-up view depicting the first section 200l′ and the second section 200l″ of the O2 sensor 200l and the lingual frenulum 118. The first section 200l′ may comprise a light emitter 256 and the second section 200l″ may comprise a light detector 258. In an embodiment, the light emitter 256 may pass a beam of light 257 through the lingual frenulum 118 and the beam of light may be measured by the light detector 258 to determine O2 concentration within the blood of the user 1. The O2 sensor 200l may operate in a manner which is similar to other O2 sensors located outside of the body of the user 1, e.g. a finger-mounted O2 sensor, by measuring the degree to which oxygenated blood absorbs light.

O2 concentration data from the sensor 200l is sent to the microprocessor 14, which may utilize the data to apply one or more therapeutic protocols such as mandibular 106 advancement, vibrational stimulation, electrical stimulation or airflow stimulation.

FIG. 3d is an embodiment of a close-up view of portion of the O2 sensor 200l. In an embodiment, there is a temperature sensor 200m on both the first section 200l′ and the second section 200l″ of the O2 sensor 200l. In an embodiment, the temperature sensor 200m may take the form of a sleeve fitting over the first section 200l′ and/or the second section 200l″ of the O2 sensor 200l. In other embodiments, the temperature sensor may comprise any other suitable shape. As depicted in FIG. 3b, the temperature sensor 200m may be located below the bottom surface of the tongue 120. The temperature sensor 200m is capable of measuring body temperature of the user 1. Measurement of user body temperature is a component in the determination of user 1 sleep state; e.g., rapid eye movement (“REM”) sleep versus non rapid eye movement sleep, and may be utilized by the microprocessor 14 to determine therapy protocol.

FIG. 3e is an embodiment of a top view of the bottom member 6 depicting the manner in which the top surface of the tongue 122 may contact the vibrational band 382 and the electrical stimulation apparatus 380.

FIG. 3f is an embodiment of a cross-sectional view of the oral therapy device 2 comprising the top member 4 and the bottom member 6 and an airflow stimulation apparatus. A small tube 362 may extend through the top member 4. A first portion of the tube 362a may extend from an exterior surface 4a of the top member 4. In an embodiment, the first portion of the tube 362a may comprise a length of between about 5 and 10 millimeters from the exterior surface 4a. A second portion of the tube 362b, may be embedded within the top member 4. An electrical fan 366 may be embedded within the second portion of the tube 362b, and may be located adjacent to a port 364 that is located at a terminal end of the top member 4. The fan 366 is capable of drawing air 363 inward through the tube 362 in a direction 368. The air 367 may be expelled through the port 364. The port 364 may comprise a flap 365 which closes during user 1 exhalation to avoid pushing saliva into the tube 362. The air being expelled 367 from the port 364 at the end of the tube 362 may be capable of inducing the surface 119 of the portion 107 of the base of the tongue 109 to move forward away from the posterior pharynx 113 increasing the area through which air may freely be drawn into the lungs (as further described in FIG. 3i).

FIG. 3g is an embodiment of a cross-sectional sagittal view of the mid-face and neck area of a user 1 wherein the oral therapy device 2 has been positioned at least partially within the oral cavity 100 of the user 1. The vibrational stimulation band 382 of the oral therapy device 2 may provide tactile stimulation to the top surface of the tongue 122 (see FIG. 3a) and may induce the surface 119 of the portion 107 of the base of the tongue 109 to move forward away from the posterior pharynx 113 (under the direction of the microprocessor 14) thus increasing the area through which air may freely be drawn into the lungs (as described in FIG. 3a). In an embodiment, a front portion of the base of the tongue 109 may remain in a pre-therapy position.

In an embodiment, a pre-therapy position of the surface 119 of the portion 107 of the base of the tongue 109 may comprise a first position 124 (dotted line). In an embodiment, an active vibrational therapy position of the surface 119 of the portion 107 of the base of the tongue 109 may comprise a second position 128 (solid line). Note that active vibrational therapy delivery by the oral therapy device 2 does not change the pre-therapy positions of the mentum of the chin 114, the upper teeth 102 or the lower teeth 104. There is a range of distance 126 between the first position 124 and the second position 128 of the surface 119 of the portion 107 of the base of the tongue 109. This range of distance 126 may be between about 0.5 centimeters and 1 centimeter, in an embodiment. The distance range 126 by which the surface 119 of the portion 107 of the base of the tongue 109 is moved away from the posterior pharynx 113 during vibrational therapy will vary from its original pre-therapy position 124 among various users 1 based upon anatomical differences. The vibrational band 382 may also aid in pulling the base of the tongue 109 forward during mandibular 106 advancement therapy, as described in FIG. 1a. Actuator 386 shown in FIG. 3a, will apply the vibrational therapy as determined by the therapy protocol management system in conjunction with the microprocessor (to be described more fully herein)

FIG. 3h is an embodiment of a cross-sectional sagittal view of the mid-face and neck area of a user 1 wherein an oral therapy device 2 has been positioned within the oral cavity 100 of the user. The oral therapy device 2 is capable of providing electrical stimulation by the electrical stimulation apparatus 380 to the top surface of the tongue 122 inducing surface 119 of the portion 107 of the base of the tongue 109 to move forward away from the posterior pharynx 113 increasing the area through which air may freely be drawn into the lungs. Anatomical pre-therapy position of the surface 119 of the portion 107 of the base of the tongue 109 is depicted as a dotted line. For example, the pre-therapy position of the surface 119 of the portion 107 of the base of the tongue 109 may comprise a first position 130. A solid line depicts an active therapy position of the surface 119 of the portion 107 of the base of the tongue 109. For example, the active therapy position of the surface 119 of the portion 107 of the base of the tongue 109 may comprise a second position 134. Note that electrical stimulation therapy delivery by the oral therapy device 2 does not change the pre-therapy positions of the mentum of the chin 114, the upper teeth 102 or the lower teeth 104. There is a range of distance 132 between the first position 130 and the second position 134 of the surface 119 of the portion 107 of the base of the tongue 109. This range of distance 132 may be potentially 1 centimeter. The distance range 132 by which the surface 119 of the portion 107 of the base of the tongue 109 is pulled away from the posterior pharynx 113 during electrical stimulation therapy will vary from its original pre-therapy position 130 among various users 1 based upon anatomical differences. Actuators 380a, 380b as shown in FIG. 3a, will apply the electrical stimulation therapy as determined by the therapy protocol management system in conjunction with the microprocessor (to be described more fully herein)

The technology described within this application provides for the provision of electrical stimulation therapy to the top surface of the tongue 122 requiring no surgical intervention and is deliverable through a removable oral therapy device 2.

FIG. 3i is an embodiment of a cross-sectional sagittal view of the mid-face and neck area of a user 1 wherein an oral therapy device 2 has been positioned within the oral cavity 100 of the user. The oral therapy device 2 is capable of providing airflow stimulation as described in FIG. 3f to the portion of the oropharynx 108 inducing the surface 119 of the portion 107 of the base of the tongue 109 to move forward and away from the posterior pharynx 113 increasing the area through which air may freely be drawn into the lungs. Anatomical pre-therapy position of the surface 119 of the portion 107 of the base of the tongue 109 is depicted as a dotted line. For example, the pre-therapy position of the surface 119 of the portion 107 of the base of the tongue 109 may comprise a first position 136. A solid line depicts an active maximum therapy position of the surface 119 of the portion 107 of the base of the tongue 109. For example, the active therapy position of the surface 119 of the portion 107 of the base of the tongue 109 may comprise a second position 140. Note that active airflow stimulation therapy delivery by the oral therapy device 2 does not change the pre-therapy positions of the mentum of the chin 114, the upper teeth 102 or the lower teeth 104. There is a range of distance 138 between the first position 136 and the second position 140 of the surface 119 of the portion 107 of the base of the tongue 109. This range of distance 138 may be potentially 1 centimeter, in an embodiment. The distance range 138 by which the surface 119 of the portion 107 of the base of the tongue 109 is pulled away from the posterior pharynx 113 during airflow stimulation therapy will vary from its original pre-therapy position 136 among various users 1 based upon anatomical differences. Actuator 366 as shown in FIG. 3f, will apply the airflow stimulation therapy as determined by the therapy protocol management system in conjunction with the microprocessor (to be described more fully herein)

The technology described herein requires no facial interface with airflow stimulation therapy being delivered directly by a removable oral therapy device.

FIG. 3j is an embodiment of a sagittal view of the mid-face and neck area of a user 1 wherein an oral therapy device 2, has been positioned within the oral cavity 100 of the user 1. As described in FIG. 1a, the oral therapy device 2 is capable of providing micro-adaptive hands-free mandibular 106 positioning therapy. The bottom member 6 of the oral therapy device 2 is capable of moving forward in response to signals received from an integral microprocessor 14 located within the oral therapy device 2.

an embodiment, movement of the bottom member 6 induces advancement of the user's 1 mandible 106 and the mentum of the chin 114 to a more-forward position 152. This therapeutic capability increases the cross-sectional area of the portion of the user's 1 oropharynx 108 potentially mitigating sleep-related breathing disorders by pulling the surface 119 of the portion 107 of the base of the tongue 109 away from the posterior pharynx 113 increasing the area through which air may freely be drawn into the lungs. However, in this embodiment, advancement of the bottom member 6 of the oral therapy device 2 has not resulted in sufficient opening of the portion of the oropharynx 108 to permit free airflow to the lungs. (See FIG. 3k depicting close-up cross-sectional sagittal view of the surface 119 of the portion 107 of the base of the tongue 109 and surrounding anatomical structures). Anatomical pre-therapy positions of the bottom member 6 of the oral therapy device 2, the surface 119 of the portion 107 of the base of the tongue 109 and the mentum of the chin 114 are depicted as dotted lines. For example, the pre-therapy position of the bottom member 6 may comprise a first position 88, the surface 119 of the portion 107 of the base of the tongue 109 may comprise a first position 142, and the mentum of the chin 114 may comprise a first position 148. Solid lines depict the active therapy positions of the aforementioned anatomical structures. For example, the active therapy position of the bottom member 6 may comprise a second position 90, the surface 119 of the portion 107 of the base of the tongue 109 may comprise a second position 144 and the mentum of the chin may comprise a second position 152. Note that mandibular 106 advancement effected by the oral therapy device 2 results in a Class 3 occlusion with an underbite position of the upper 102 and lower teeth 104. There is a distance 89 between the first position 88 and second position 90 of the bottom member 6. This distance 89 may potentially be approximately 1 centimeter, in an embodiment. There is a distance 150 between the first 148 and second 152 position of the mentum of the chin 114 that is equivalent and directly related to the distance between the first position 88 and second position 90 of the bottom member 6. Detailed illustration of the effect of mandibular 106 advancement in this embodiment is shown in detail in FIG. 3k.

FIG. 3k is an embodiment of a close-up cross-sectional sagittal view of the portion 107 of base of the tongue 109 and surrounding anatomical structures during active mandibular 106 advancement therapy as described in the embodiment illustrated in FIG. 3j. The surface 119 of the portion 107 of the base of the tongue 109 may comprise a first position 142 and the surface 119 of the portion 107 of the base of the tongue 109 may comprise a second position 144. In an embodiment, a distance 145 is the overall lateral dimension of the portion of the oropharynx 108 during active therapy. There is a distance 143 between the first 142 position and the second 144 position of the surface 119 of the portion 107 of the base of the tongue 109 that may be potentially less than the corresponding distances of the bottom member 6 and the mentum of the chin 114 as shown in FIG. 3j. This difference may be due to the elasticity of the portion 107 of the base of the tongue 109 and the base of the tongue 109 and other surrounding anatomical structures. As noted in FIG. 1a the distance 143 by which the surface 119 of the portion 107 of the base of the tongue 109 is moved away from the posterior pharynx 113 during therapy will vary from its original pre-therapy position 142 among various users 1 based upon anatomical differences.

In an embodiment, the overall lateral dimension of the portion of the oropharynx 108 resulting from mandibular advancement therapy provided by the oral therapy device 2 is insufficient to enable a free airflow to the lungs. The oral therapy device 2 has the capability to provide various multiple therapies, i.e., mandibular advancement therapy, vibrational stimulation therapy, electrical stimulation therapy and airflow stimulation therapy in a simultaneous manner. FIGS. 3l-3n illustrate multiple embodiments of the oral therapy device 2 providing mandibular positioning therapy in augmentation with vibrational stimulation, electrical stimulation and airflow stimulation therapies.

FIG. 3l is an embodiment of a cross-sectional sagittal view of the mid-face and neck area of a user 1 wherein an oral therapy device 2, has been positioned at least partially within the oral cavity 100 of the user 1. FIGS. 3j-3k previously illustrated the effects of mandibular 106 advancement therapy delivery by the oral therapy device 2 upon the surface 119 of the portion 107 of the base of the tongue 109, the base of the tongue 109 and surrounding anatomical structures. In an embodiment, while mandibular 106 advancement therapy has moved the surface 119 of the portion 107 of the base of the tongue 109 away from the posterior pharynx 113 to a point 144, further therapy may potentially be required to provide additional distance between the surface 119 of the portion 107 of the base of the tongue 109 and the posterior pharynx 113 through which air may be freely drawn into the lungs. FIG. 3l illustrates an embodiment of augmentation of active mandibular 106 advancement therapy with vibrational stimulation by the vibrational band 382 of oral therapy device 2 to the top surface of the tongue 122. In an embodiment, anatomical pre-vibrational stimulation therapy position of the surface 119 of the portion 107 of the base of the tongue 109 is depicted as a dotted line. For example, the pre-vibrational stimulation therapy position of the surface 119 of the portion 107 of the base of the tongue 109 may comprise a first position 144. Solid lines depict the active vibrational stimulation therapy position of the surface 119 of the portion 107 of the base of the tongue 109. For example, the active vibrational stimulation therapy position of the surface 119 of the portion 107 of the base of the tongue 109 may comprise a second position 147. There is a distance 146 between the first position 144 and second position 147 of the surface 119 of the portion 107 of the base of the tongue 109. This distance 146 may potentially be 0.5 centimeter, in some embodiments. This distance range 146 may vary among users based upon anatomical differences. There is a distance 154 between the second position 147 of the surface 119 of the portion 107 of the base of the tongue 109 and the posterior pharynx 113. The distance 154 may be about 1.0 centimeter, in an embodiment. The distance 154 may vary among users 1 based upon anatomical differences. Note that the active mandibular advancement therapy position 90 of the bottom member 6 of the oral therapy device 2 and the active mandibular advancement therapy position 152 of the mentum of the chin 114 do not change during augmentation through provision of vibrational stimulation therapy by the oral therapy device 2 to the top surface of the tongue 122.

FIG. 3m is an embodiment of a cross-sectional sagittal view of the mid-face and neck area of a user 1 wherein an oral therapy device 2, has been positioned at least partially within the oral cavity 100 of the user 1. In an embodiment, while mandibular 106 advancement therapy has moved the surface of the portion 107 of the base of the tongue 109 to a point 144, further therapy may potentially be required to provide additional distance between the surface 119 of the portion 107 of the base of the tongue 109 and the posterior pharynx 113 through which air may be freely drawn into the lungs. FIG. 3m illustrates an embodiment of augmentation of active mandibular 106 advancement therapy by the electrical stimulation apparatus 380 of the oral therapy device 2 to the top surface of the tongue 122. In an embodiment, anatomical pre-electrical stimulation therapy position of the surface 119 of the portion 107 of the base of the tongue 109 is depicted as a dotted line. For example, the pre-electrical stimulation therapy position of the surface 119 of the portion 107 of the base of the tongue 109 may comprise a first position 144. Solid lines depict the active electrical stimulation therapy position of the surface 119 of the portion 107 of the base of the tongue 109. For example, the active electrical stimulation therapy position of the surface 119 of the portion 107 of the base of the tongue 109 may comprise a second position 156.

There is a distance 155 between the first position 144 and second position 156 of the surface 119 of the portion 107 of the base of the tongue 109. This range 155 maybe about 0.5 centimeter in an embodiment. This distance 155 may potentially vary among various users 1 based upon anatomical differences. There is a distance 157 between the second position 156 of the surface 119 of the portion 107 of the base of the tongue 109 and the posterior pharynx 113. The distance 157 may be about 1.0 centimeter in an embodiment. The distance 157 may potentially vary among users 1 based upon anatomical differences. Note that the active mandibular 106 advancement therapy position 90 of the bottom member 6 of the oral therapy device 2 and the active mandibular 106 advancement therapy position 152 of the mentum of the chin 114 do not change during augmentation through provision of electrical stimulation therapy by the oral therapy device 2 to the top surface of the tongue 122

FIG. 3n is an embodiment of a cross-sectional sagittal view of the mid-face and neck area of a user 1 wherein an oral therapy device 2, has been at least partially positioned within the oral cavity 100 of the user 1. In an embodiment, while mandibular advancement therapy has moved the surface 119 of the portion 107 of the base of the tongue 109 to a point 144, further therapy may potentially be required to provide additional distance between the surface 119 of the portion 107 of the base of the tongue 109 and the posterior pharynx 113 through which air may be freely drawn into the lungs. FIG. 3n illustrates an embodiment of the augmentation of active mandibular 106 advancement therapy with airflow stimulation therapy as described in FIG. 3f by the fan 366 drawing air 363 into the oral therapy device 2 through the airflow tube 362 and expelling air 367 adjacent to the top surface of the tongue 122 and the surface 119 of the portion 107 of the base of the tongue 109. In an embodiment, anatomical pre-airflow stimulation therapy position of the surface 119 of the portion 107 of the base of the tongue 109 is depicted as a dotted line. For example, the pre-airflow stimulation therapy position of the surface 119 of the portion 107 of the base of the tongue 109 may comprise a first position 144. Solid lines depict the augmentation of airflow stimulation therapy position of the surface 119 of the portion 107 of the base of the tongue 109. For example, through augmentation, the active airflow stimulation therapy position of the surface 119 of the portion 107 of the base of the tongue 109 may comprise a second position 159.

There is a distance 158 between the first position 144 and second position 159 of the surface 119 of the portion 107 of the base of the tongue 109. This distance 158 may be 0.5 centimeter in an embodiment. This distance 158 may potentially vary from its original pre-therapy position 144 among various users 1 based upon anatomical differences. There is a distance range 160 between the second position 159 of the surface 119 of the portion 107 of the base of the tongue 109 and the posterior pharynx 113. The distance 160 may be about 1.0 centimeter in an embodiment. The distance 160 may vary among users 1 based upon anatomical differences. Note that the active mandibular 106 advancement therapy position 90 of the bottom member 6 of the oral therapy device 2 and the active mandibular advancement therapy position 152 of the mentum of the chin 114 do not change during augmentation through provision of airflow stimulation therapy by the oral therapy device 2 to the top surface of the tongue 122 and to the surface 119 of the portion 107 of the base of the tongue 109.

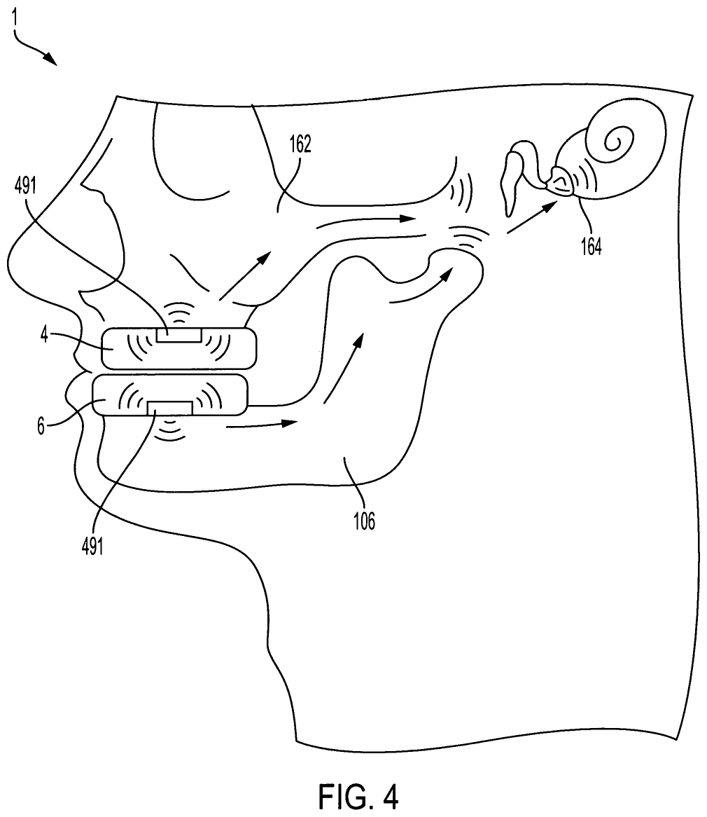

FIG. 4 is an embodiment of a sagittal view of the mid-face and neck area of a user 1 wherein the oral therapy device 2, has been at least partially positioned within the oral cavity 100 of the user 1. The oral therapy device 2 has the capability to provide tinnitus/insomnia therapy. Sound is produced by one or more speakers 491 at least partially embedded within the top member 4 and/or bottom member 6 of the oral therapy device 2. A range of vibrational frequency between about 6 kHz to about 20 kHz in some embodiments, may be produced by the one or more speakers 491, with a corresponding range of sound intensity of between about 0 to about 90 decibels. The high frequency energy produced by the one or more speakers 491 may travel through the bony structures of either the mandible 106 and/or the maxilla 162 reaching the inner auditory ear canal 164. The high frequency energy produced by the one or more speakers 491 within the oral therapy device 2 may mask the annoyance of tinnitus symptoms and improve brain function. In addition, the sound produced by the speakers 491 may further serve as “white noise” mitigating user 1 insomnia.

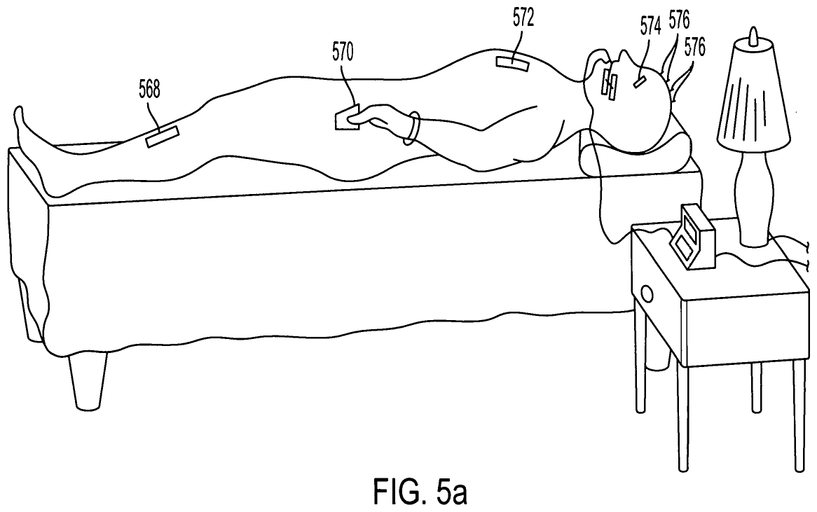

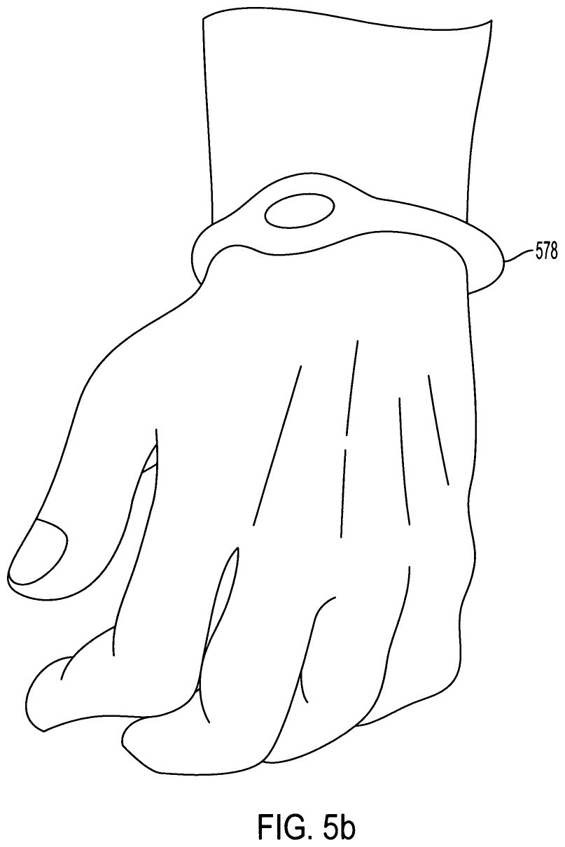

FIG. 5a depicts an embodiment of collaborative sensors (sensors 568-576) that may be utilized in analysis of the user 1 sleep state. The data procured by these collaborative sensors may be sent to logic devices within the base station, wherein the collaborative sensor data is accessible by the microprocessor 14 within the oral therapy device 2. In an embodiment, the collaborative sensor data is utilized by the microprocessor 14 to further optimize therapy protocol regimen that is available for the user 1 to further combat sleep disordered breathing The collaborative sensors include, but are not limited to, muscle tension sensor 568, blood oxygen (O2) concentration sensor 570, chest effort sensor 572, rapid eye movement (“REM”) sensor 574 and brain wave sensor 576. Collaborative sensors include any sensors which are located outside the confines of the oral therapy device 2.

FIG. 5b depicts an embodiment of a collaborative movement sensor 578, which may be located on the user's 1 wrist. In other embodiments, the collaborative movement sensor 578 may be located in any other suitable location depending on the particular application.

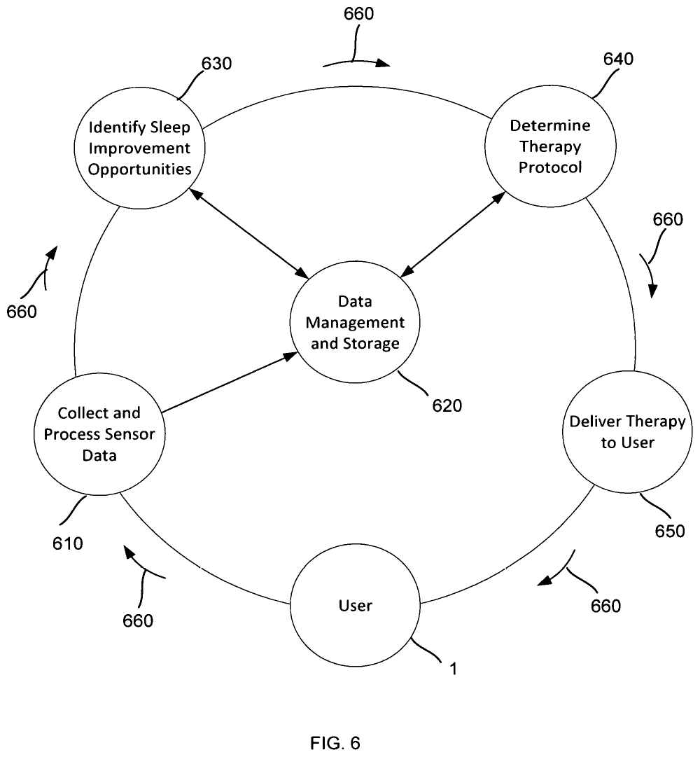

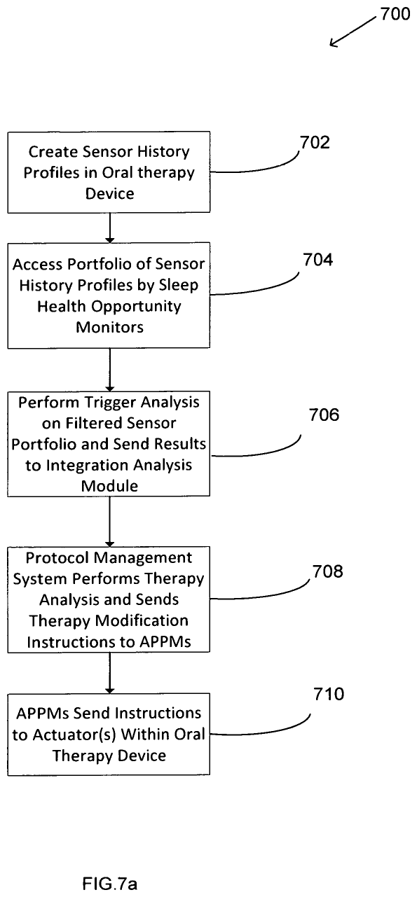

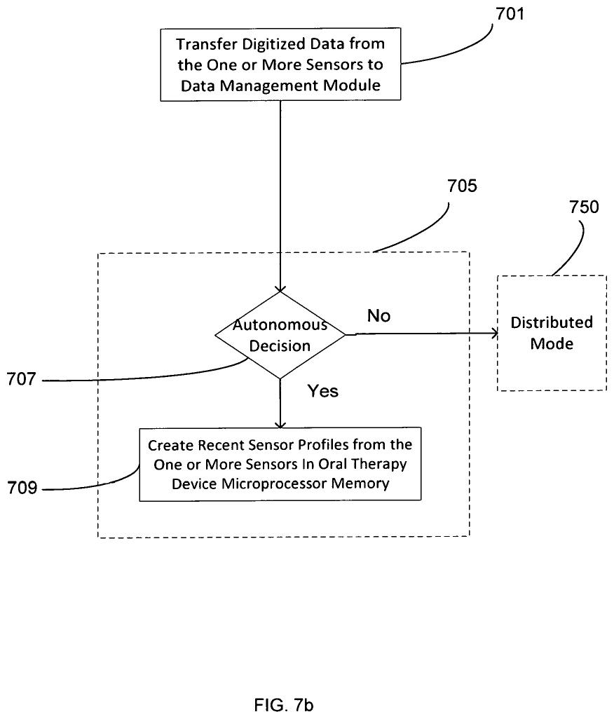

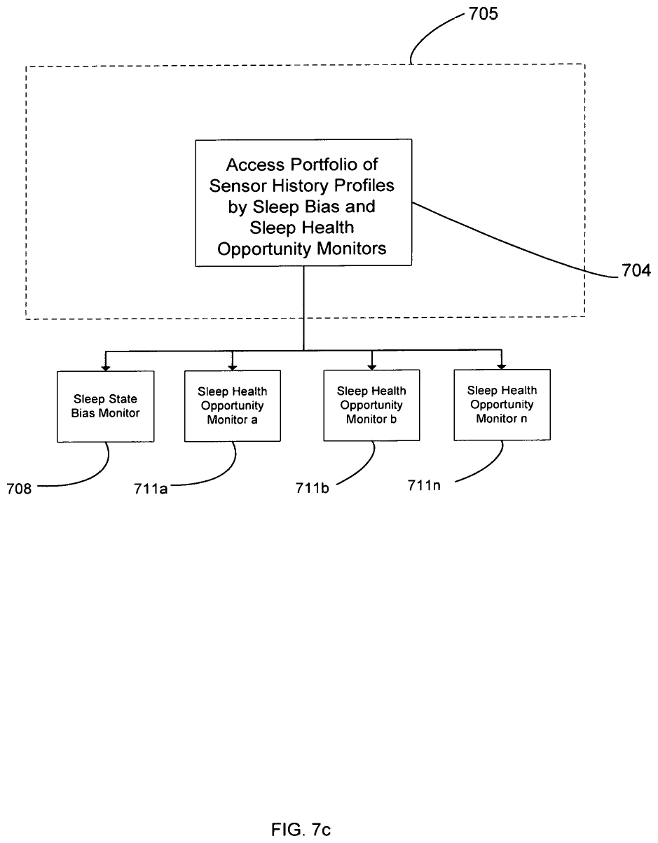

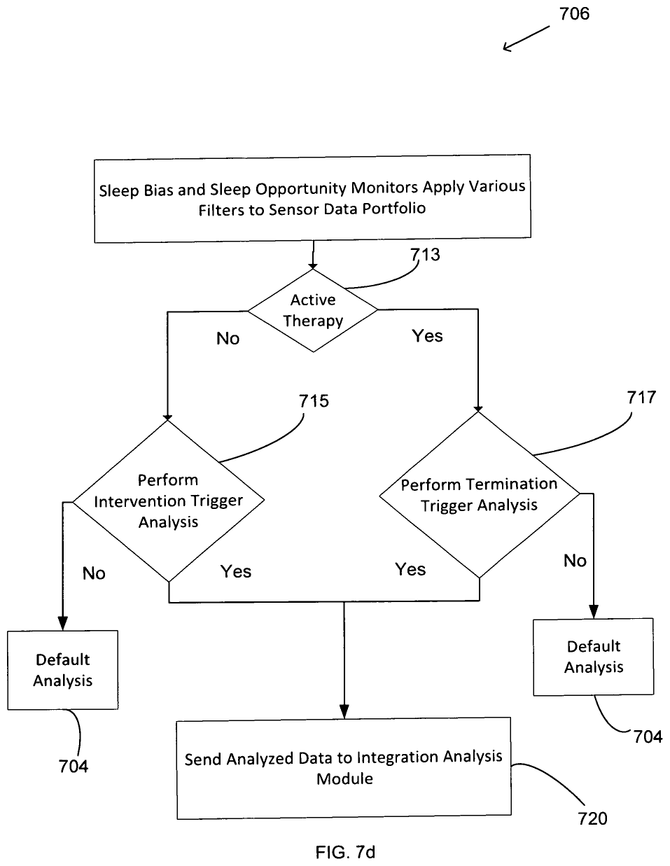

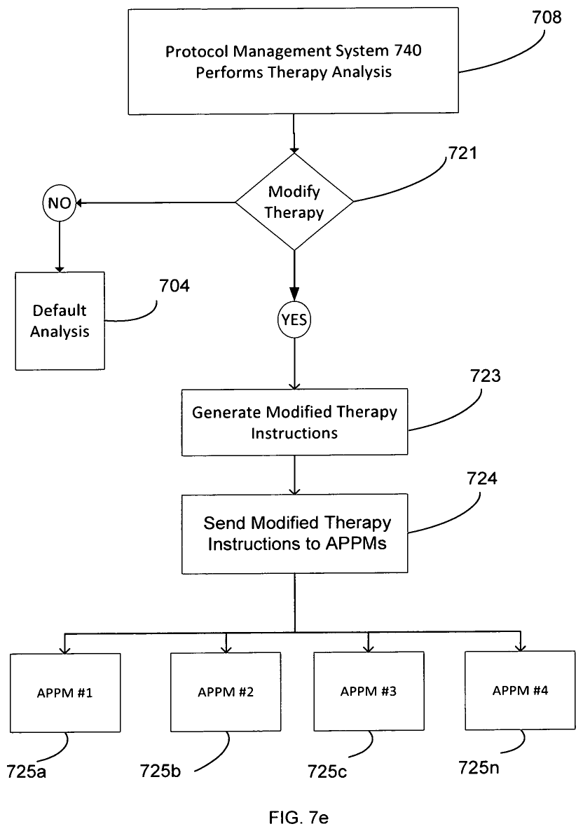

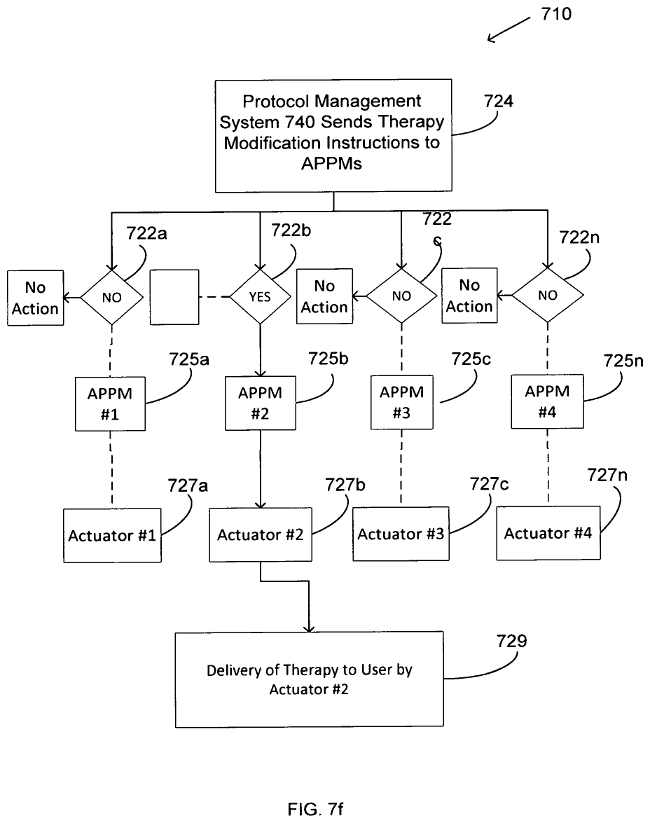

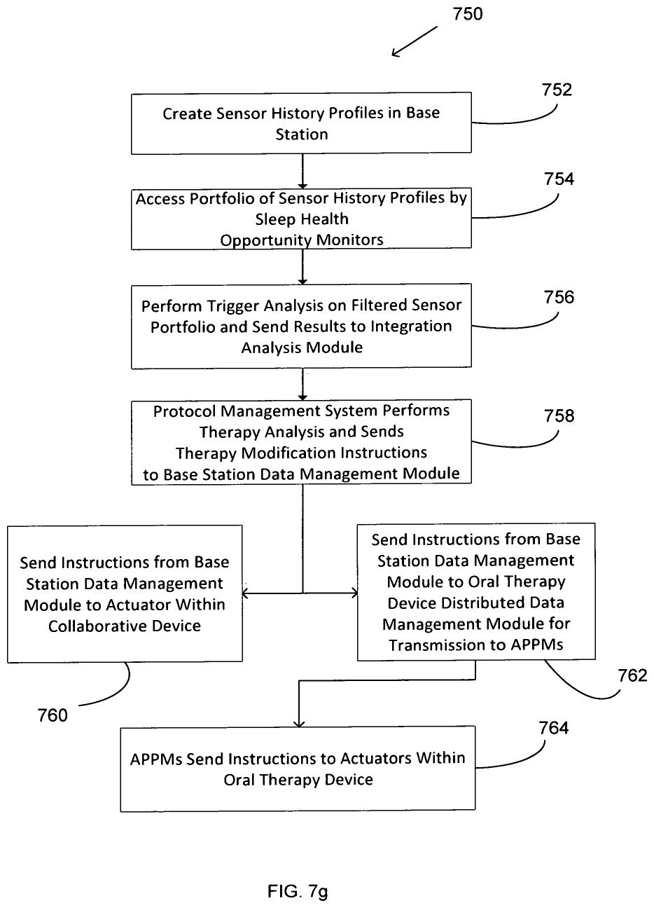

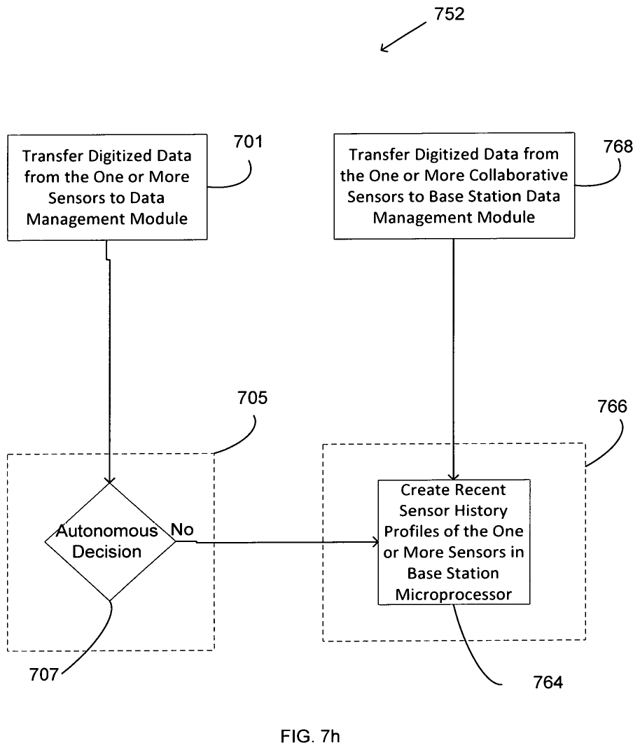

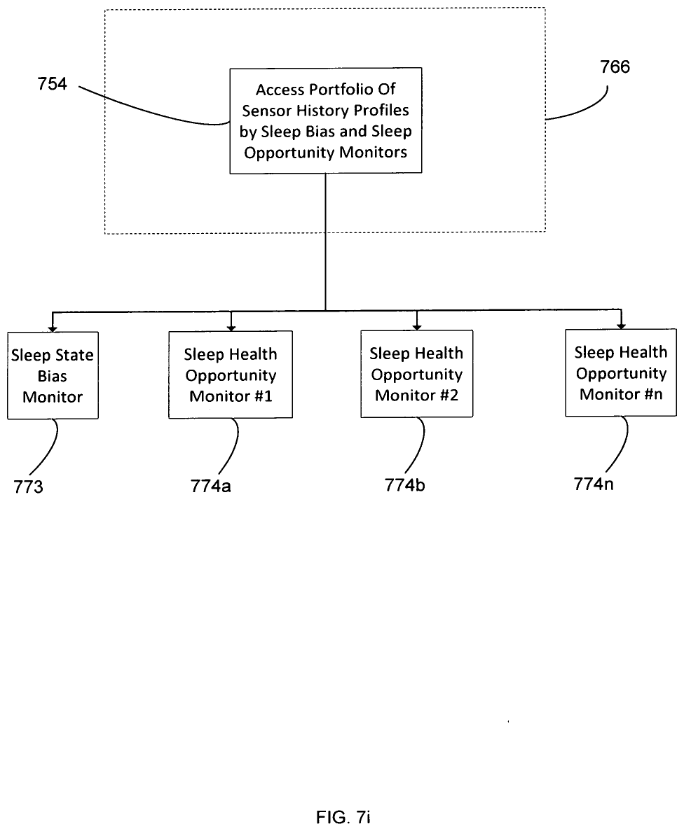

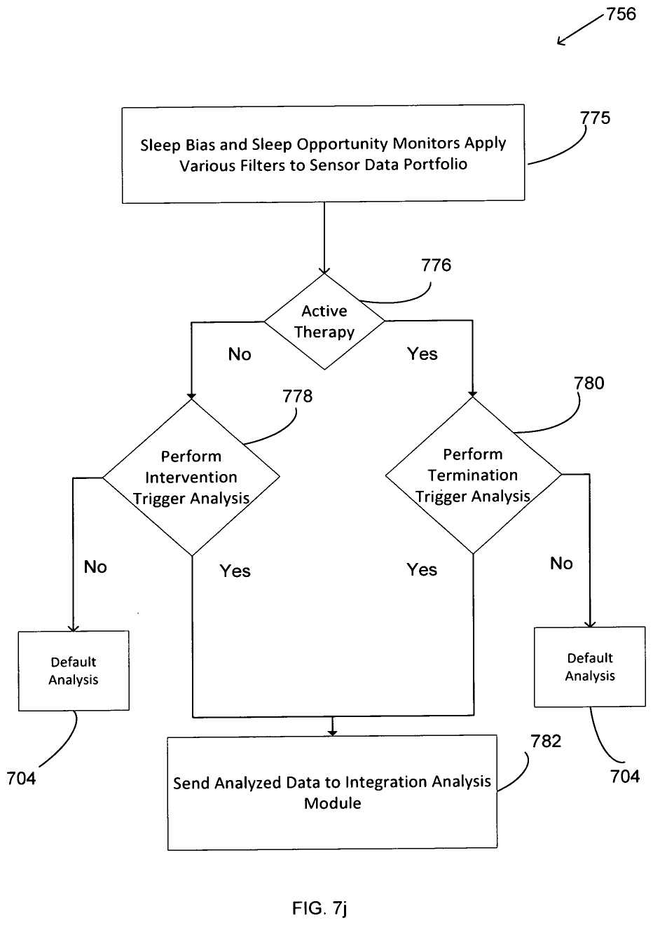

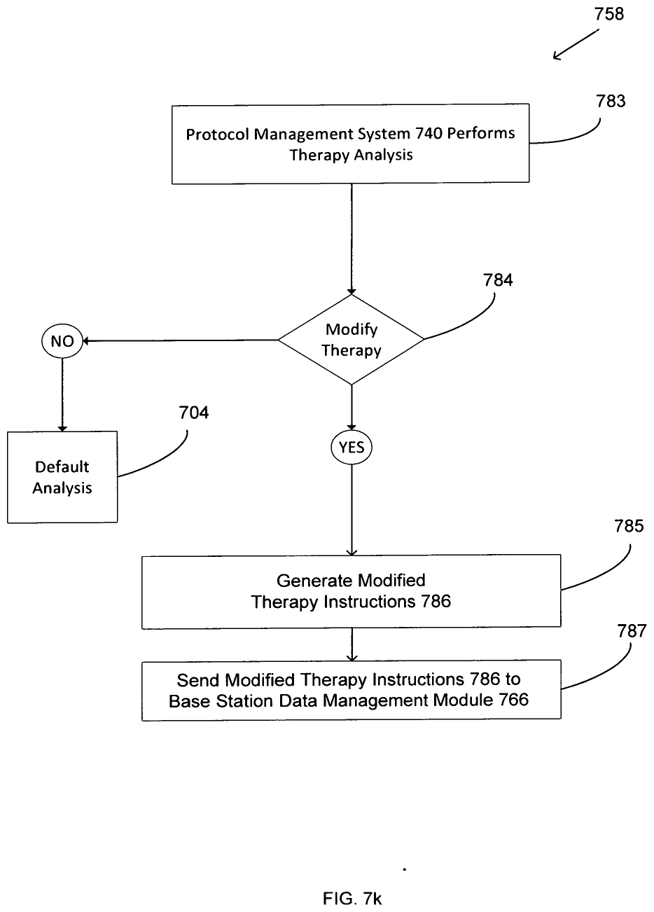

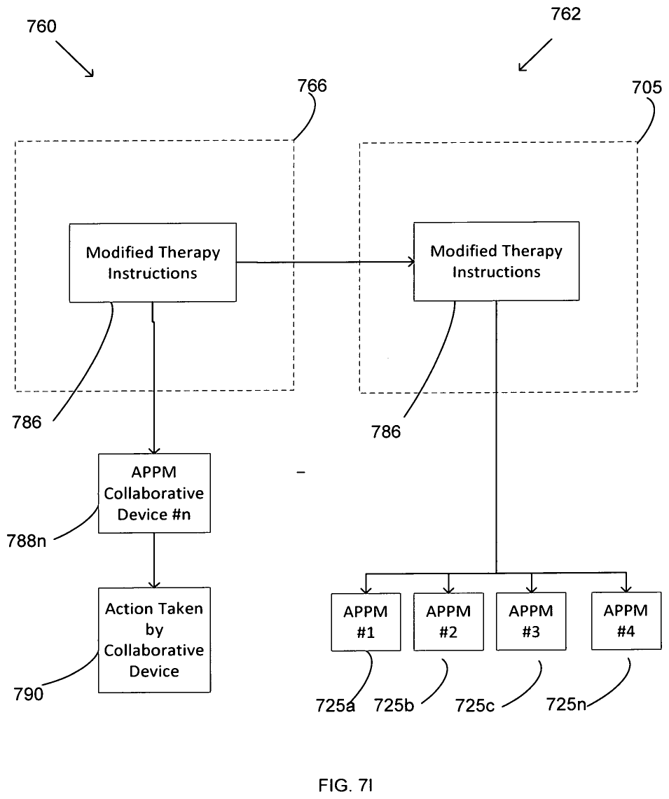

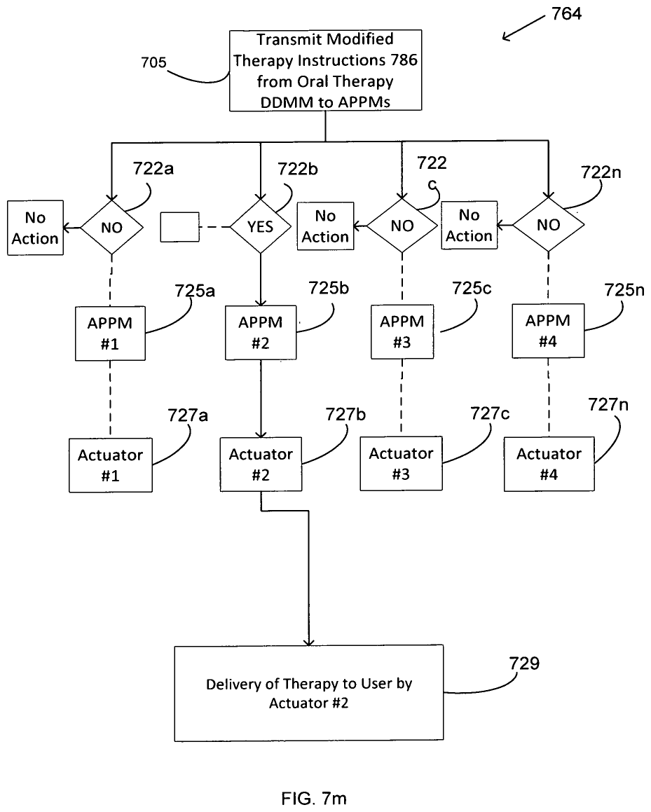

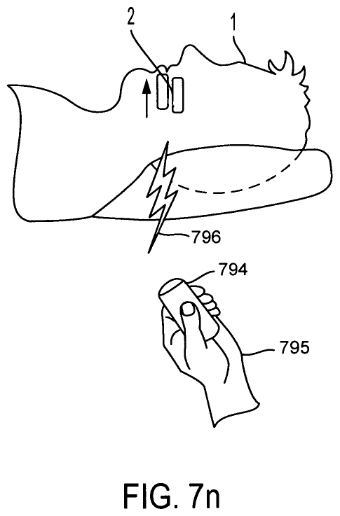

FIG. 6 broadly depicts an embodiment of the operational process of use of the oral therapy device 2 by a user 1. The various phases of the operational process are more fully described in FIGS. 7a-7n. The principal aspects of the operational process may comprise the collection and analysis of user sensory data, the identification of sleep improvement opportunities, the determination of therapy optimization, and the delivery of such therapy by the oral therapy device. Various data are stored and accessed throughout the operational process. At operation 610, real time sensor data relative to the user's 1 sleep and other health parameters is collected from the one or more sensors described in FIGS. 2, 5a and 5b and processed. The processed sensor data is then forwarded to a data management and storage module 620. At operation 630, selected sensor data are accessed from the data management and storage module 620 and analyzed by one or more software modules to identify sleep improvement opportunities. If a sleep improvement opportunity is identified, at operation 640, a Protocol Management System (PMS) (to be further described herein) accesses and analyzes selected sensory and therapy protocol data from the data storage and management module 620 to optimize the user's 1 sleep quality. Following analysis of the accessed data, if the PMS determines that provision of therapy or revision of current therapy will improve the user's 1 sleep quality, the PMS may determine the nature and duration of such therapy. The PMS may then forward therapy instructions to various modules within the oral therapy device 2 instructing actuators to deliver such therapy. A copy of such therapy instructions is forwarded by the PMS to the data management and storage module 620. At operation 650, the oral therapy device 2 carries out the therapy instructions received from the PMS and delivers the instructed therapy to the user 1. The process repeats in a clockwise direction 660 with the collection and processing of real-time user 1 sensory data, analysis of such data and modification of therapeutic protocol.

FIG. 7a depicts an embodiment of autonomous operational mode. When the oral therapy device 2 is in use within the oral cavity 100 of the user 1, it has two distinct operational modes; autonomous and distributed. FIG. 7a provides a generalized view of autonomous mode 700, for which specific details of each operation will be further described herein. While in autonomous mode 700, the oral therapy device 2 operates on a stand-alone basis and may interact with componentry internal to its physical structure. At operation 702, sensory history profiles (which are a compilation of recent sensor data history) are created from raw sensory data retrieved from sensors (as depicted in FIG. 2) located within the oral therapy device 2. At operation 704, a portfolio comprised of some or all sensor history profiles may be accessed by one or more sleep health opportunity monitors (SHOM), which are located within the microprocessor 14 memory. At operation 706, the SHOMs filter the sensor history profile data, perform trigger analysis, and send the filtered and analyzed data to a Protocol Management System (PMS) within the microprocessor 14. The PMS processes information forwarded from each SHOM utilizing a customized protocol for each specific user. At operation 708, the PMS performs therapy analysis and therapy modification decisions are made. Therapy instructions are then forwarded to activation profile management modules (APMMs). Each APMM may be electrically coupled to an actuator, wherein the actuator physically controls therapy delivery by the oral therapy device. At operation 710, instructions are sent to the one or more actuators, and therapy instructions are physically carried out by the oral therapy device 2.

FIG. 7b depicts an embodiment of operation 702 in more detail. In an embodiment, one or more sensors (such as any of those depicted in FIG. 2) may send analog or digital sensor data gathered during monitoring of the user, to an analog to digital converter (ADC) within the microprocessor 14. The ADC converts any analog data to digital. The digital data is then forwarded at operation 701 to a data management module (DMM) 705. At operation 707 the DMM 705 performs a decision as to whether or not the oral therapy device 2 is operating in autonomous or distributed mode. If the oral therapy device is operating in distributed mode, operation 750 begins within the base station (see FIG. 10a, 10b) external to the oral therapy device 2. If the oral therapy device 2 is operating in autonomous mode, the DMM 705 creates and stores the sensor data into a recent sensor history profile 709 located within the oral therapy device microprocessor memory 16.

FIG. 7c depicts an embodiment of operation 704 in further detail. In an embodiment, a recent sensor history portfolio may be comprised of data from the one or more sensor data history profiles. The recent sensor history portfolio may be accessed from the microprocessor memory 16 via the DMM 705 by each of the one or more SHOM 711a-711n and by a sleep state bias monitor (SSBM) 708. Each SHOM 711a-711n and the SSBM 708 may monitor a particular aspect of the user's sleep state and status.

FIG. 7d depicts an embodiment of operation 706 in further detail. In an embodiment, each SHOM 711a-711n and the SSBM 708 may apply various statistical filters to the recent sensor history portfolio data and may thus emphasize different aspects of the data to assess a particular aspect of the user's sleep state. In an embodiment, sleep stages such as Rapid Eye Movement (REM) sleep and non-REM sleep may influence the nature and duration of therapy delivered by the oral therapy device 2, an auditory user snoring level or user O2 concentration measurement may influence the nature and duration of therapy delivered by the oral therapy device 2. At operation 713 a yes or no decision is made by each SHOM 711a-711n regarding active therapy status. If therapy is not active, at operation 715 a determination is made as to whether or not an intervention trigger threshold has been reached by each specific SHOM 711a-711n. If an intervention threshold has not been met at operation 715, default analysis comprising operation 704 continues. If, however, an intervention threshold has been met at operation 715, an affirmative signal is sent to a Integration Analysis Module (“IAM”) 720 along with all filtered recent sensor history portfolio data from the SHOMs 711a-711n and the SSBM 708 for therapy analysis. If therapy is active at operation 713, filtered recent sensor history portfolio data is utilized at operation 717 to determine whether or not a therapy termination trigger has been reached. If a termination trigger has been reached at operation 717, an affirmative message is sent to the IAM 720 along with all filtered recent sensor history portfolio data for further analysis. If at termination trigger has not been reached at operation 717, no message is sent to the IAM 720 and default analysis comprising operation 704 continues.

FIG. 7e depicts an embodiment of operation 708 in further detail. In an embodiment, the IAM 720 may process filtered recent sensor history portfolio data utilizing a user-specific Protocol Management System (PMS) 740. The PMS 740 is a collection of software modules that work together to determine if and when therapy is warranted and then determines the most appropriate therapy or combination of therapies to correspond to the user's sleep state. The nature and delivery of therapeutic intervention by the oral therapy device is determined and governed through a process of continuous analysis of sensory data regarding the user's sleep state and sleep quality by the PMS 740. The PMS 740 may utilize various statistical and mathematical analyses to optimize therapy delivery by the oral therapy device to the user. The PMS 740 may analyze and evaluate the user's current sleep state and sleep quality in relation to the current therapy being delivered by the oral therapy device. The PMS 740 may continuously assess the user's sleep state and quality throughout the sleep session and adjust therapy as needed to optimize outcomes. The PMS 740 may learn which therapeutic interventions are the most successful in specific instances for each individual user 1.

Following analysis of the user's current sleep state and sleep quality in relation to the current therapy being delivered by the oral therapy device, the PMS 740 may decide at operation 721 that therapy optimization is indicated. If therapy optimization is indicated, modification of current therapy being provided by the oral therapy device 2 may be required. If the PMS 740 decides to modify current therapy at operation 721, the PMS 740 generates modified therapy instructions at operation 723 specifying the rate, intensity, duration and termination limit for the selected therapy(ies). At operation 724 modified therapy instructions are sent to one or more activation profile management modules (“APMMs”) 725a, 725b, 725c, 725n, located within the oral therapy device 2. If, however, the PMS 740 decides at operation 721 that therapy modification is not required, then current therapy being delivered by the oral therapy device 2 is continued and default analysis comprising operation 704 continues.

FIG. 7f depicts an embodiment of operation 710 in greater detail. In an embodiment, at operation 724 modified therapy instructions are forwarded to one or more APMMs 725a-725n. Instructions contain specific instructions for the delivery of therapy by the oral therapy device 2. At operation 722a-722n an action/no action decision enables the transmission of instructions from the PMS 740 to only those one or more APMMs 725a-725n in which therapy modification is required. Each APMM 725a-725n is electrically coupled to a specific therapy actuator 727a-727n within the oral therapy device. Hence, there may be a specific APMM/therapy actuator set for mandibular advancement therapy, electrical stimulation therapy, vibrational stimulation therapy and airflow stimulation therapy, etc. In an embodiment, the therapy modification instructions sent from the PMS 740 to the APMMs 725a-725n direct the oral therapy device 2 to provide mandibular advancement therapy. In this embodiment, the instructions may direct the APMM 725b to initiate modified mandibular advancement therapy comprising the advancement distance, the rate of advancement, the duration of advancement, etc. The modified therapy instructions are sent from the APMM 725b to its specific therapy actuator 727b, wherein the actuator 727b causes the specified therapy to be delivered by the oral therapy device 2 to the user 1 at operation 729.

In FIG. 7g, an embodiment is shown wherein a distributed operational mode 750 of the oral therapy device 2 is described. FIG. 7g provides a generalized view of distributed mode 750, for which specific details of each operation will be further described herein. While in distributed mode 750, the oral therapy device 2 interacts with componentry internal to its physical structure and with componentry internal to a base station (described more fully in FIGS. 10a-10b). In distributed mode, communication and data processing capabilities are enhanced due to the increased processing power available within the base station. While in distributed mode, both the DMM 705 and the PMS 740 within the oral therapy device 2 become Distributed Data Management System (“DDMS”) and Distributed Protocol Management System (“DPMS”) denoting their distributed relationship with the base station componentry. The processing of data from collaborative sensors 568-578 is enabled only while operating in distributed mode.

At operation 752, sensory history profiles (which are a compilation of recent sensor data history) are created within the base station microprocessor from raw sensory data retrieved from sensors 200a-200m located within the oral therapy device 2 and from collaborative sensors 568-578 external to the oral therapy device 2. At operation 754, the sensor history profiles may be accessed by one or more sleep health opportunity monitors (SHOM) and by a Sleep Bias Monitor. At operation 756, the SHOMs may filter the sensor history profile data, perform trigger analysis, and send the filtered and analyzed data to a customized protocol management system (PMS) 740 specific to each individual user and located within the base station microprocessor. At operation 758, the PMS 740 performs therapy analysis and therapy modification decisions are made and therapy instructions forwarded to a base station data management module. At operation 760, therapy instructions are sent to one or more collaborative devices by the base station data management module. At operation 762, instructions are sent to the oral therapy device distributed data management module by the base station data management module for transmission to APMMs within the oral therapy device 2. Each APMM may be electrically coupled to an actuator, wherein the actuator physically controls therapy delivery. At operation 764, instructions are sent from the APMMs to one or more actuators, and therapy instructions are physically carried out by the oral therapy device 2.

FIG. 7h depicts an embodiment of operation 752 in greater detail. In an embodiment, one or more sensors (such as any of those depicted in FIG. 2) may send analog or digital sensor data gathered during monitoring of the user, to an analog to digital converter (ADC) within the microprocessor. The ADC converts any analog data to digital. At operation 701, the digital data is forwarded to a data management module (DMM) 705 within the oral therapy device 2. At operation 707 the DMM 705 performs a decision as to whether or not the oral therapy device 2 is operating in autonomous or distributed mode. If the oral therapy device is operating in distributed mode and not autonomous mode, operation 752 begins within the base station (see FIG. 10a, 10b) external to the oral therapy device 2. At operation 764, a microprocessor located within the base station creates and stores recent sensor history profiles. In an embodiment, at operation 768, digitized data from one or more collaborative sensors (such as those depicted in FIGS. 5a,5b) is forwarded to the base station DMM 766 and recent sensor history profiles are created and stored in memory at operation 764 by the base station microprocessor.

FIG. 7i depicts an embodiment of operation 754 in further detail. In an embodiment, a recent sensor history portfolio may be comprised of data from the one or more sensor data history profiles. The recent sensor history portfolio may be accessed at operation 754 via the base station DMM 766 by each of the one or more SHOM 774a-774n and by a sleep state bias monitor (SSBM) 773. Each SHOM 774a-774n and the SSBM 773 may monitor a particular aspect of the user's sleep state and status.

FIG. 7j depicts an embodiment of operation 756 in further detail. In an embodiment, at operation 775 each SHOM 774a-774n and the SSBM 773 may apply various statistical filters to the recent sensor history portfolio data and may thus emphasize different aspects of the data to assess a particular aspect of the user's sleep state. In an embodiment, the SSBM 773 may assess sleep stages such as Rapid Eye Movement (REM) sleep and non-REM sleep that may influence the nature and duration of therapy delivered by the oral therapy device, In an embodiment, the SHOMs 774a-774n may assess auditory user snoring levels or user O2 concentration measurements that may influence the nature and duration of therapy delivered by the oral therapy device. At operation 776 a yes or no decision is made by each SHOM 774a-774n regarding active therapy status. If therapy is not active, at operation 778 a determination is made as to whether or not an intervention trigger threshold has been reached by each specific SHOM 774a-774n. If an intervention threshold has not been met at operation 778, default analysis comprising operation 704 continues. If, however, an intervention threshold has been met at operation 778, an affirmative signal is sent to the IAM 782 along with all filtered recent sensor history portfolio data from the SHOMs 774a-774n and the SSBM 773 for therapy analysis. If therapy is active at operation 776, filtered recent sensor history portfolio data is utilized at operation 780 to determine whether or not a therapy termination trigger has been reached. If a termination trigger has been reached at operation 780, an affirmative message is sent to the IAM 782 along with all filtered recent sensor history portfolio data from the SHOMs 774a-774n and the SSBM 773 for further analysis. If a termination trigger has not been reached at operation 780, no message is sent to the IAM 782 and default analysis comprising operation 704 continues.

FIG. 7k depicts an embodiment of operation 758 in further detail. In an embodiment, at operation 783 the IAM 782 may process filtered recent sensor history portfolio data utilizing a user-specific protocol management system (PMS) 740. The PMS 740 may utilize various statistical and mathematical analyses to optimize therapy delivery by the oral therapy device 2 to the user. The PMS 740 may analyze and evaluate the user's current sleep state and sleep quality in relation to the current therapy being delivered by the oral therapy device. Following analysis of the user's current sleep state and sleep quality in relation to the current therapy being delivered by the oral therapy device 2, at operation 784 the PMS 740 may decide that therapy optimization is indicated. If therapy optimization is indicated at operation 784, modification of current therapy being provided by the oral therapy device 2 is required. At operation 785 the PMS 740 generates modified therapy instructions 786 specifying the rate, intensity, duration and termination limit for the selected therapy(ies). At operation 787 these modified therapy instructions 786 are sent to the base station data management module 766. If, however, the PMS 740 decides therapy modification is not required at operation 784, then current therapy being delivered by the oral therapy device 2 is continued and default analysis comprising operation 704 continues.

FIG. 7l depicts an embodiment of operations 760 and 762 in greater detail. In an embodiment, at operation 760, modified therapy instructions 786 are sent by the base station DMM 766 to APMMs 788n for the one or more collaborative devices. At operation 790 therapy delivery is performed by the one or more collaborative devices. Simultaneously, at operation 762, modified therapy instructions 786 are forwarded by the base station DMM 766 to the oral therapy device DDMM 705 and then forwarded to the one or more APMMs 725a-725n within the oral therapy device.

FIG. 7m depicts an embodiment of operation 764 in greater detail. In an embodiment, modified therapy instructions 786 are sent by the oral therapy device DDMM 705 to only those one or more APMMs 725a-725n within the oral therapy device 2 where therapy modification is required. Each APMM 725a-725n is electrically coupled to a specific therapy actuator 727a-727n within the oral therapy device 2. Hence, there may be a specific APMM/therapy actuator set for mandibular advancement therapy, electrical stimulation therapy, vibrational stimulation therapy and airflow stimulation therapy, etc. At operation 722a-722n a yes or no decision is made regarding action required by each APMM 725a-725n. If action is required, the APMM 725a-725n will accordingly signal the actuator 727a-727n. In an embodiment, the therapy modification instructions sent from the oral therapy device DDMM 705 to the APMM 725 causes the actuator 727b to provide the specified mandibular advancement therapy. In an embodiment, these instructions may direct the APMM 725b to initiate modified mandibular advancement therapy comprising the advancement distance, the rate of advancement, the duration of advancement, etc. at operation 729.

FIG. 7n depicts an embodiment of activation of therapeutic intervention by the oral therapy device 2 through usage of a remote device 794. In an embodiment, a bed partner 795 causes an intervention request 796 to be initiated through a remote device 794. Such request may be evaluated by the PMS 740 within the oral therapy device microprocessor and evaluated along with sensory data for analysis and therapy delivery to the user 1 by the oral therapy device 2.

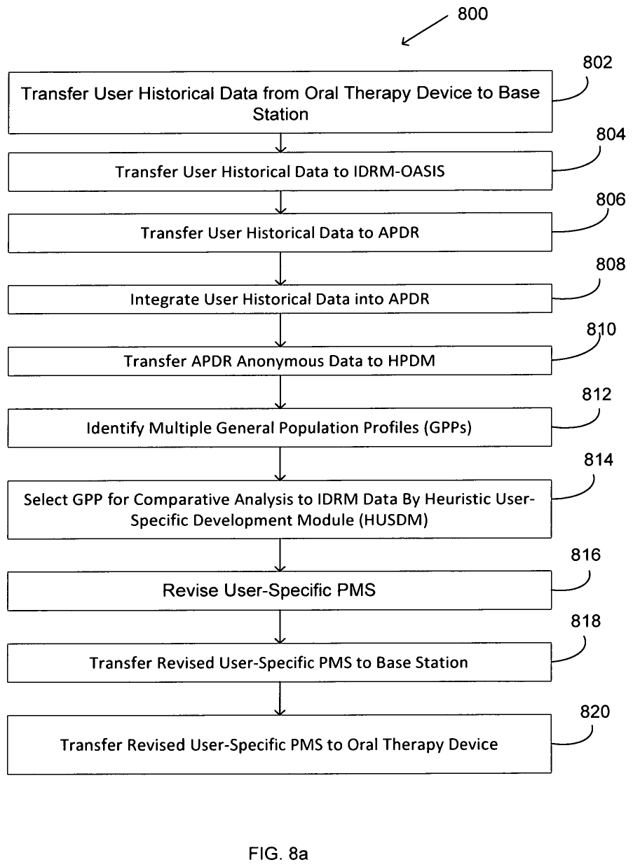

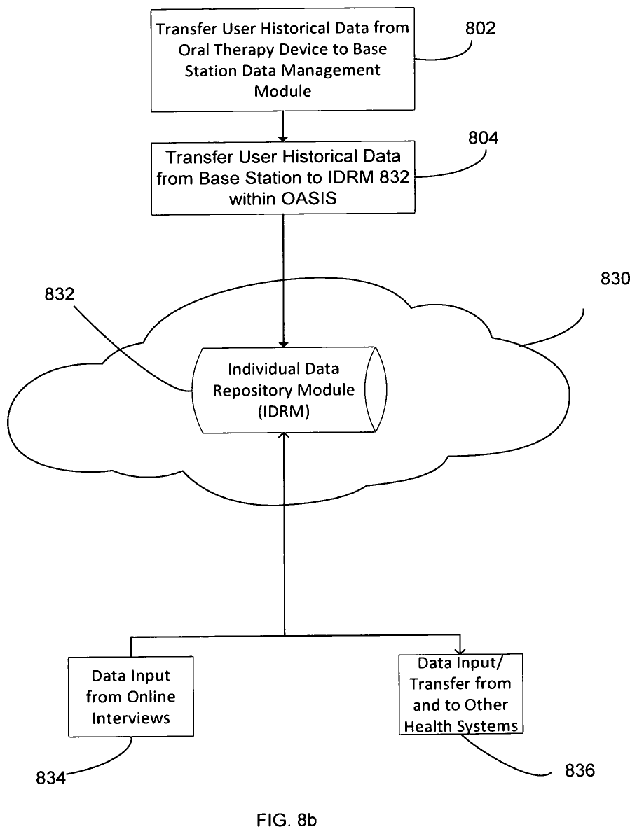

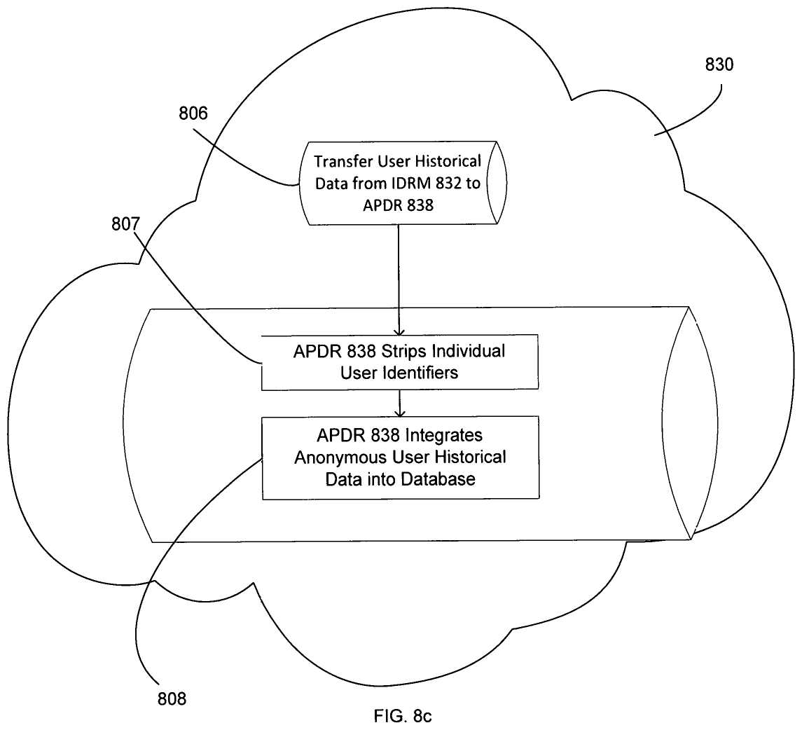

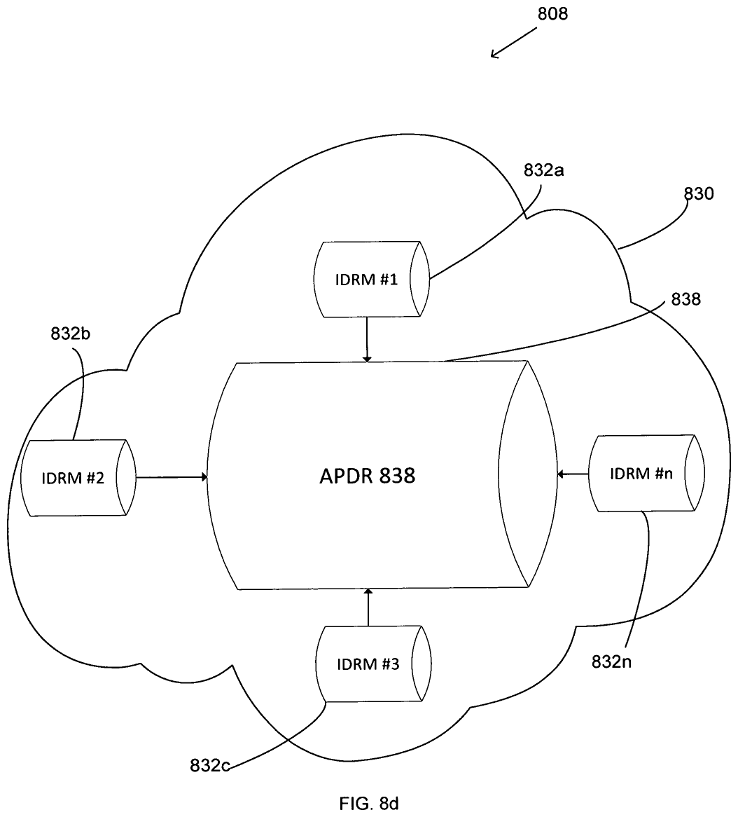

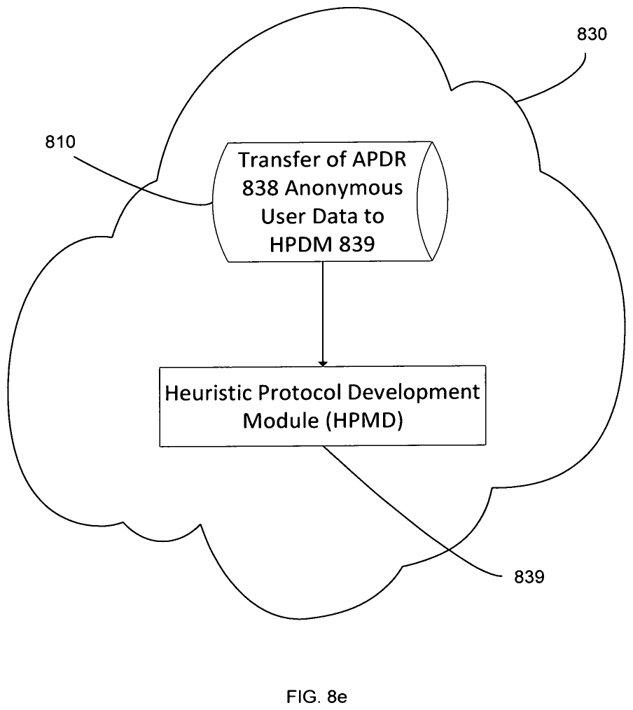

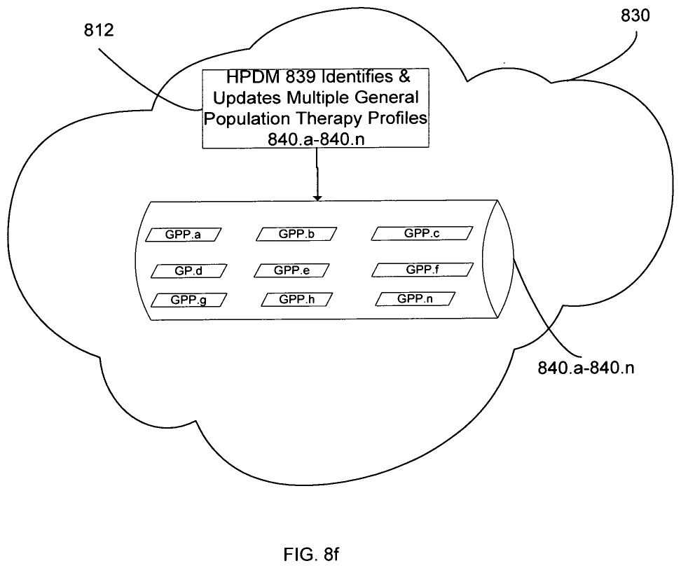

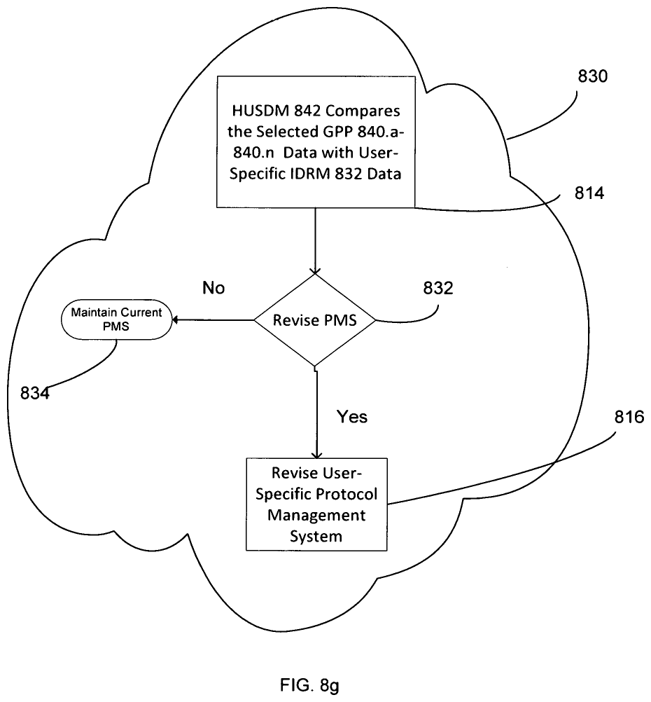

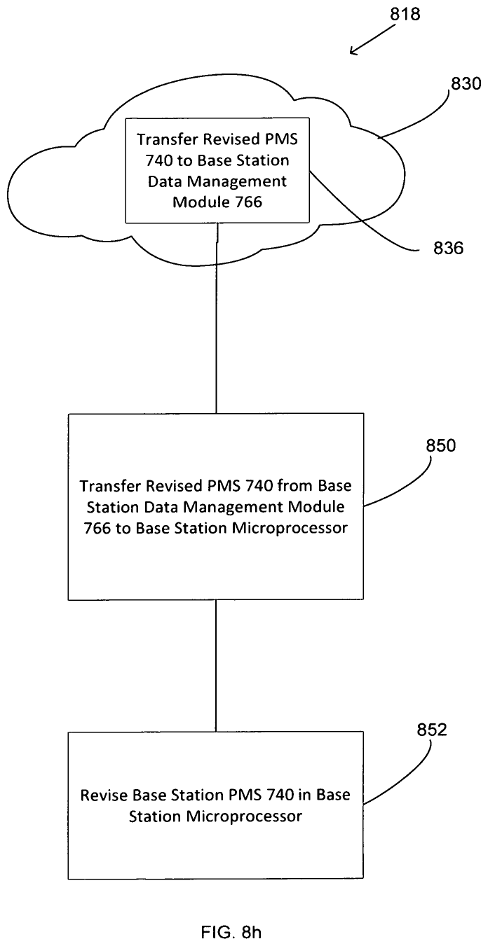

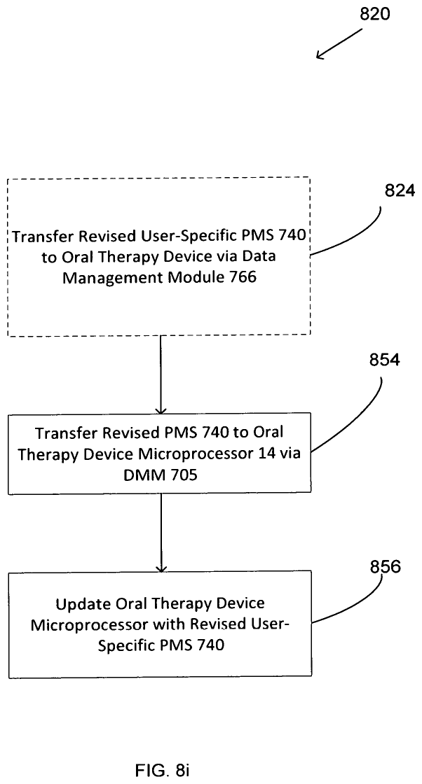

In FIG. 8a, an embodiment is shown wherein the oral therapy device 2 is acting in a data-exchange mode with a cloud-based Obstructive Apnea Support and Information System (OASIS). FIG. 8a provides a generalized view of data-exchange mode, for which specific details of each operation will be further described herein. In an embodiment, when in data-exchange mode, the oral therapy device and the base station may be in communication with OASIS, a cloud-based series of software programs that aggregates anonymous summary user data and intelligently analyzes such data seeking to identify therapy trends to continuously improve outcomes for the totality of the user base. Through big-data analysis tools and techniques, OASIS may optimize each user's 1 specific therapy protocol based upon therapeutic outcomes derived from a base of multiple users. Modification of a specific user's therapy protocol may involve revision of the current PMS residing in the microprocessors of both the base station and the oral therapy device of the specific user.

At operation 802, user historical data is transferred from the oral therapy device 2 to the base station. At operation 804 user historical data is transferred from the base station to an Individual Data Repository Module (IDRM) within OASIS. In addition, other relevant data may be input directly to the IDRM from online interviews and other health management systems. At operation 806, user data is transferred from the IDRM to an Anonymous Population Data Repository (APDR). At operation 808 all user identifiers are stripped and all anonymous user data is integrated into a database within the APDR. At operation 810, the APDR database is transferred to a Heuristic Protocol Development Module (HPDM). At operation 812, multiple general population profiles (GPPs) are identified by the HPDM from the APDR database. At operation 814, a Heuristic User-Specific Development Module (HUSDM) compares outcomes data from a selected GPP with a selected individual user's data from their IDRM. At operation 816, the HUSDM may determine whether optimization of the selected user's PMS 740 is warranted. If optimization is warranted, the HUSDM may accordingly revise the selected user's current PMS 740 to improve therapeutic outcome for the selected user. At operation 818, the selected user's revised PMS 740 is transferred from the HUSDM through the OASIS cloud to the selected user's base station microprocessor wherein the revised PMS 740 replaces the previous version. At operation 820, the selected user's base station sends the revised PMS 740 to the microprocessor 14 within the selected user's oral therapy device 2 wherein the revised PMS 740 replaces the previous version.

FIG. 8b depicts an embodiment of operations 802 and 804 in greater detail. In an embodiment, at operation 802 user historical data is transferred from the oral therapy device 2 storage memory 16 to the base station (see FIG. 10a, 10b) storage memory. This user historical data may include recent sleep history, filtered and analyzed sensory portfolio data, therapy delivered by the oral therapy and resulting outcomes. At operation 804, the user historical data is transferred from the base station storage memory to the IDRM 832 within the cloud-based OASIS system 830. Each IDRM 832 within OASIS 830 may be specific for each user and may serve as the repository for all individual user data utilized by OASIS. The IDRM 832 may further collect user-specific demographics that may influence the user's 1 susceptibility to negative health factors and responsiveness to therapeutic interventions directed by the treatment protocol. This user-specific demographic information may be continuously updated and may be obtained directly from the user via online interviews at operation 834. Relevant user health data may be accessed from and transferred to other medical information systems at operation 836. This data transfer from OASIS 830 to other health management systems may provide for the analysis of patient data and correlation with other health measurements may reduce the need for patients to undergo expensive and time-consuming polysomnographic testing and allow for the cost-effective treatment of patients who snore and monitor their progression to screen for sleep apnea reducing the need for patients to visit physicians and other healthcare providers.

FIG. 8c depicts an embodiment of operations 806 and 808 in greater detail. In an embodiment, at operation 806 the IDRM 832 transfers the user's historical data to the APDR 838. At operation 807, the APDR 838 strips all individual user identifiers making the identity of the user anonymous. At operation 808, the anonymous user data is integrated into a database.

FIG. 8d depicts an embodiment of operation 808 in greater detail. At operation 808, the APDR 838 may integrate the data from one or more user-specific IDRMs 832a-832n into a multi-user database comprised of data from the one or more anonymous users.

FIG. 8e depicts an embodiment of operation 810 in greater detail. In an embodiment, anonymous data from the one or more users comprising the APDR 838 database may be transferred to the HPDM 839.

FIG. 8f depicts an embodiment of operation 812 in greater detail. In an embodiment, at operation 812, the HPDM 839 may utilize factor analysis and other techniques to identify and update general population profiles (GPPs) 840.a-840.n from the multiple user database within the APDR 838. These GPPs (GPP.a-GPP.n) 840.a-840.n may be segmented and identified into groups of similar age, weight, neck size, history of diagnosed sleep disorders, etc. The identification and delineation of these groups GPP.a-GPP.n (840.a-840.n) may be meaningful in the analysis of therapeutic outcomes among users. Further, compilation and analysis of data via age, race, gender, etc., may be utilized in overall therapeutic planning and outcomes analysis on a national basis. In addition, assessment of baseline mandibular positioning and measurements relative to patient-specific anatomical features and relative distances may be relevant in treatment protocols.

FIG. 8g depicts an embodiment of operation 814 and operation 816 in greater detail. In an embodiment, the HUSDM 842 may select a specific anonymous user for analysis and access the relevant user data from its user-specific IDRM 832. The HUSDM 842 may then select a specific GPP 840.a-840.n from the HPDM 839 that is most closely associated with that specific user. At operation 814, the HUSDM 842 will then compare and analyze the historical data and therapeutic outcomes from the specific user IDRM against similar data and therapeutic outcomes from the most closely associated GPP 840a.-840.n. This analysis may determine and quantify differences in outcomes between the user and their associated GPP 840a-840n. Heuristic user-specific development is a process that compares and contrasts individual user data and aggregate population data. This analysis may further identify potential revisions in the specific user's PMS 740 which may result in improved outcomes for the specific user. At operation 832, a yes or no decision is made as to whether revision in the current user's PMS 740 may by warranted to improve therapeutic outcomes. If the decision is made that no revision is warranted, at operation 834 the current PMS 740 for the user is maintained. If the decision is made to modify the existing PMS 740, then at operation 816 the HUSDM 842 makes the necessary revisions to the current user PMS 740.

FIG. 8h depicts an embodiment of operation 818 in greater detail. In an embodiment, at operation 836 the revised user-specific PMS 740 is transferred from the OASIS cloud 830 to the user's base station DMM 766. A copy of the revised user-specific PMS 740 is also transferred to the user's 1 IDRM 832 within the OASIS cloud 830. At operation 850, the revised user-specific PMS 740 is transferred via the base station DMM 766 to the base station microprocessor. At operation 852, the revised user-specific PMS 740 replaces the previous version of the PMS 740 within the base station microprocessor.

FIG. 8i depicts an embodiment of operation 820 in greater detail. In an embodiment, at operation 824, the revised user-specific PMS 740 is transferred from the base station microprocessor via the DMM 766 to the DMM 705 within the oral therapy device 2. At operation 854, the revised user-specific PMS 740 is transferred by the oral therapy DMM 705 to the microprocessor 14 within the oral therapy device 2. At operation 856, the microprocessor 14 within the oral therapy device 2 replaces the previous version of the user-specific PMS 740 with the revised version.

FIG. 9a depicts an embodiment of a cross-sectional sagittal view of the mid-face and neck area of a user 1 showing the relative normal positioning of the mentum of the chin 114, the mandible 106, the upper teeth 102, the lower teeth 104 and the region of the Temporomandibular joint (“TMJ”) 972. The TMJ 972 is formed by the articulation of the condyle 973 of the mandible 106 and the temporal bone 987 of the cranium. It is located anteriorly to the inner auditory ear canal 164, on the lateral aspect of the face. The TMJ 972 is a complex encapsulated anatomical structure whose main function is to provide for smooth pain-free movement of the mandible 106 for eating, speaking and other normal human activities. There is an at-rest baseline distance 986 between the lateral-edge head 982 of the condyle 973 of the mandible 106 and lateral edge of the external auditory ear canal 164. This at-rest baseline distance may be from about 10 mm to about 25 mm. In an embodiment, during sleep, the cross-sectional area of the portion of the user's oropharynx 108 is reduced due to the close proximity of the soft palate 168, the uvula 170, and/or the posterior pharynx 113 to the surface 119 of the portion 107 of the base of the tongue 109. This reduction in cross-sectional area of the portion of the oropharynx 108 may result in sleep disordered breathing.

FIG. 9b depicts an embodiment wherein advancement of the user's 1 mandible 106 is shown in a direction 990, such as that which may result through usage of a traditional oral appliance. Mandibular 106 advancement therapy has been shown to be effective in treating SBD by pulling the surface 119 of the portion 107 of the base of the tongue 109 further away from the posterior pharynx 113 thus increasing the cross-sectional area of the portion of the oropharynx 108 increasing airflow to the lungs. The distance by which the mandible 106 is advanced causes a corresponding increase in the distance by which the lateral-edge head 982 of the condyle 973 of the mandible 106 is pulled away from its normal at-rest position adjacent to the external auditory ear canal 164. Advancement of the mandible 106 in a direction 990 and corresponding advancement of the lateral edge head 982 of the condyle 973 results in a displacement of the at-rest internal anatomy of the TMJ 972. See FIGS. 9c-9e for detailed descriptions of the resulting displacement. Displacement of the TMJ 972 for a relatively brief period of time normally results in minimal risk to the user 1. However, current oral appliances used to advance the user's mandible 106 are static in nature and do not move from a set position during the entire sleep session without human intervention. Hence, usage of current oral appliances results in displacement of the at-rest internal anatomy of the TMJ 972 throughout the user's entire sleep session. Periodic retraction of the mandible as directed by the microprocessor and effected by the oral therapy device 2 may mitigate strain and potential damage to the TMJ 972.

The greater the distance by which the mandible 106 is advanced in the direction 990, the greater the degree to which the cross-sectional area of the portion of the oropharynx 108 is increased leading to a reduction in the symptoms of SDB. However, the greater the distance of mandibular 106 advancement in the direction 990, the greater the degree of displacement within the at-rest internal anatomy of the TMJ. In an embodiment, for each 1.0 centimeter of advancement of the mandible 106 in a direction 990, an increase of 1.0 centimeter or less in the sagittal dimension of the portion of the oropharynx 108 (as shown in FIG. 1a) will result, thus increasing cross-sectional area. However, each 1.0-centimeter advancement of the mandible 106 in a direction 990 will result in a 1.0-centimeter displacement in the at-rest internal anatomy of the TMJ 972. In current practice, for some users, it is virtually impossible to advance the mandible 106 far enough in a direction 990 to mitigate SBD symptoms for the entire sleep session without causing stress on the internal anatomy of the TMJ 972. TMJ 972 pain from continued displacement may be severe and if the TMJ 972 becomes diseased or damaged, surgical repair is both costly and, in most cases, unsuccessful.

FIG. 9c depicts an embodiment of a close-up cross-sectional view of the anatomical components of the TMJ 972 in a normal at-rest position. Of major importance is the articular disk 974 which is located between and moves within the interior compartment 975 and the superior compartment 976 of the TMJ 972. The interior compartment 975 is bordered by the fibrous articular tissue 978 of the head 983 of the condyle 973. The superior compartment 976 is bordered by the fibrous articular tissue 980 of the mandibular fossa 981. The location and placement of the articular disk 974 in a normal at-rest healthy TMJ 972 is precisely between the top head 983 of the condyle 973 and the articular eminence 984 of the mandibular fossa 980. There is a normal at-rest distance 979 of between 10-25 millimeters between the lateral-edge 977 of the articular disk 974 and the external auditory ear canal 164.

FIG. 9d depicts an embodiment of a sagittal close-up superimposed view of the TMJ 972 illustrating the relative positional changes of the major components of the TMJ 972 resulting from mandibular 106 advancement. The normal at-rest positions of the components of the TMJ are shown as solid lines while displaced positions are shown as dotted lines. The normal at-rest positions of the lateral-edge head 982 of the condyle 973 and the lateral-edge 977 of the articular disk 974 are shown in their normal positional relationship to the external auditory ear canal 164. The displaced position 982′ of the lateral edge head 982 of the condyle 973 and the displaced position 977′ of the lateral edge 977 of articular disk 974 due to mandibular advancement are shown as dotted lines. Mandibular advancement such as that depicted in FIG. 9b results in the condyle 973 correspondingly advancing in the direction 990 and also moving downward in the direction 992. In an embodiment, for each 1.0 centimeter of advancement of the mandible 106 in direction 990, an increase of 1.0 centimeter or less in the sagittal dimension of the portion of the oropharynx 108 (as shown in FIG. 1a) will result. However, each 1.0-centimeter advancement of the mandible 106 in direction 990 will result in a 1.0 centimeter or less advancement of the lateral head 982 of the condyle 973 and a less than 1.0-centimeter downward movement of the lower lateral edge 985 of the condyle 973 to a position 985′. The lower portion of the lateral-edge 977 of the articular disk 974 also advances in the direction 990 and moves downward in direction 992. See FIG. 9e for detailed depiction of the changes and deformity of the articular disk 974 resulting from mandibular 106 advancement. The optimal advancement distance required to improve sleep quality may be determined by the oral therapy device 2 PMS 740 based upon feedback from the sensory portfolio, a process described more fully in FIGS. 6a-6d and FIGS. 7a-7n. The distance by which the mandible 106 is advanced is periodically reduced in a controlled fashion over time by the oral therapy device 2 to mitigate potential stress on the TMJ 172 and in particular, the articular disk 974.

FIG. 9e depicts an embodiment of the change in the shape of the articular disk 974 resulting from mandibular 106 advancement from an at-rest position shown as solid lines to a displaced position shown as dotted lines. Advancement of the mandible 106 causes the lower lateral edge 997 of the articular disk 974 adjacent to the inner auditory ear canal to move in a direction 990 and a downward direction 992 to a location 997′. In addition, advancement of the mandible 106 causes the lower lateral edge on the opposite side 998 of the articular disk 974 to move in a direction 990 and an upward direction 993 to a location 998′. In an embodiment, for each 1.0 centimeter by which the mandible 106 is advanced, the distance by which the lower lateral edge 997 of the articular disk 974 adjacent to the inner auditory ear canal 164 may move in a direction 990 by 1.0 centimeter or less and may move downward in a direction 992 by 1.0 centimeter or less. Correspondingly, in an embodiment, for each 1.0 centimeter by which the mandible 106 is advanced, the distance by which the lower lateral edge 998 on the opposite side of the articular disk 974 may move in a direction 990 by 1.0 centimeter or less and may move upward in a direction 993 by 1.0 centimeter or less. The degree to which the articular disk is displaced during mandibular advancement is due, in part, to the fact that the top edge of the articular disk is attached to the fibrous articular tissue of the mandibular fossa 981 as depicted in FIGS. 9c-9d. The bottom edge of the articular disk 974 is attached to the fibrous articular tissue 978 of the head of the condyle 973 as depicted in FIGS. 9c-9d. As the mandible 106 is moved in a direction 990, the condyle 973 of the mandible 106 moves accordingly and distorts the normal shape and position of the articular disk 974.

FIG. 10a depicts an embodiment of the base station 1000 which may be utilized to charge the oral therapy device battery 10 when it is not in use, analyze data history from internal onboard sensors such as those depicted in FIG. 2 and external collaborative sensors such as those depicted in FIGS. 5a-5b and to provide a communications channel between the oral therapy device 2 and other health management systems as depicted in FIGS. 8a-8i. The base station 1000 may comprise an internal digital microprocessor 1012 and storage memory 1014 and related electronics capable of executing the base station 1000 distributed PMS and facilitating either wireless or direct communication with the oral therapy device 2 and other collaborative devices and systems through a data bus 1018. In an embodiment, the base station 1000 is positioned on an elevated surface near enough to the sleeping location of the user 1 that the oral therapy device 2 may be attached via a tethering cable 1002 to the base station 1000. In an embodiment, the tethering cable 1002 may provide a connection from the base station 1000 to the oral therapy device 2 through which humidification may be delivered to the oral cavity 100 of the user 1. In an embodiment, the tethering cable may enable communication with the oral therapy device microprocessor 14 via a data bus 1018 spanning the tethering cable 1002 and connecting to the oral therapy device data bus 18b through a tethering port connector 1004. Data may be transferred between the oral therapy device microprocessor 14 and the base station microprocessor 1012 in a direction 1005. A power source 1010 within the base station 1000 may charge the oral therapy device battery 10 in a direction 1007 via an electrical bus 1016 spanning the tethering cable 1002 and connecting to the oral therapy device electrical bus 12b through the tethering port connector 1004. The base station 1000 may connect to the internet for communication with other health management systems either wirelessly or through an ethernet connector 1008. The base station 1000 may comprise one or more electronic interface ports 1006 (such as USB) to provide direct communication with collaborative devices. Additionally, short-range wireless communication componentry 1015 within the microprocessor 1012 may enable communication with the oral therapy device 2 and/or any other wireless collaborative devices. In an embodiment, the base station 1000 may comprise a compartment 1020 for the oral therapy device 2 to receive UV radiation for sanitation treatment while not in use.

FIG. 10b depicts an embodiment of the oral therapy device 2 stowed in the UV light compartment of the base station 1020. The oral therapy device 2 has been unfolded at the attachment point 22. The contact surfaces of the top member 4 and the bottom member 6 rest on the lower glass panels 1024 with the UV lights 1022 above and below. The portions of the top member 4 and the bottom member 6 that are out of the contact plane are recognized. Additional UV light irradiates the device 2 from above 1026. UV light 1028 irradiates the top member 4 and bottom member 4 of the oral therapy device and may sanitize the surfaces.

### EXAMPLES

Example 1 is an oral therapy device comprising: a top member, wherein the top member fits over at least a portion of upper teeth within an oral cavity of a user, and wherein the top member comprises one or more at least partially embedded sensors, a bottom member, wherein the bottom member fits over at least a portion of bottom teeth within the oral cavity of the user, a coupling structure physically joining a portion of the top member to a portion of the bottom member, and a mandibular positioning drive (MPD), wherein the MPD is at least partially embedded within the bottom member, and wherein the MPD is capable of moving the bottom member from a first position to a second position.

Example 2 includes the oral therapy device of example 1 wherein a cross sectional area of an airway opening of the user is increased by the movement of the MPD.

Example 3 includes the oral therapy device of example 1 wherein a distance between the posterior pharynx and the base of the tongue is capable of being moved from a first position to a second position in response to a movement of the MPD.

Example 4 includes the oral therapy device of example 1 wherein the top member comprises at least one of a plastic or rubberized plastic-like material.

Example 5 includes the oral therapy device of example 1 wherein the bottom member further includes at least one of an electrical stimulus apparatus, a vibrational stimulus apparatus or a sound speaker.

Example 6 includes the oral therapy device of example 1 wherein the coupling structure comprises a hinge structure.

Example 7 includes the oral therapy device of example 1 wherein the first position of the MPD comprises one of an advancement or a retraction of the mandible, wherein the second position of the MPD comprises one of an advancement or retraction of the mandible.

Example 8 includes the oral therapy device of example 1 wherein the oral therapy device is capable of reducing a stress on the temporomandibular joint of the user.

Example 9 includes the oral therapy device of example 1 wherein the first position and the second position of the MPD are capable of being optimized to minimize a displacement distance of the articular disk of the temporomandibular joint, and wherein the displacement distance comprises a distance forward and a distance backward during a time period.

Example 10 includes the oral therapy device of example 1 wherein the top member further includes at least one of an embedded microprocessor, a storage memory or a battery.

Example 11 includes the oral therapy device of example 1 wherein the one or more sensors comprises at least one of an auditory sensor, an airflow sensor, a vibration sensor, an orientation sensor, or a pH sensor.

Example 12 includes the oral therapy device of example 10 wherein the one or more sensors is communicatively coupled with the microprocessor.

Example 13 includes the oral therapy device of example 10 wherein the microprocessor is capable of receiving a signal from the one or more sensors, and wherein the microprocessor is capable of checking a target value for the one or more sensors.

Example 14 includes the oral therapy device of example 10 wherein the microprocessor is capable of sending a signal to the MPD, and wherein the MPD is capable of moving the bottom member.

Example 15 includes the oral therapy device structure of example 10 wherein the microprocessor is communicatively coupled to one or more actuators that are at least partially embedded within the oral therapy device.

Example 16 includes the oral therapy device of example 15 wherein the one or more actuators include at least one of an electrical motor, a fan or an electrode.

Example 17 includes the oral therapy device of example 14 wherein an MPD actuator is physically coupled to the MPD.

Example 18 includes the oral therapy device of example 1 wherein an electrical stimulus apparatus is at least partially embedded within the bottom member.

Example 19 includes the oral therapy device of example 1 wherein the MPD comprises a microelectronic mechanical system (MEMS) that is driven by a microprocessor.

Example 20 includes the oral therapy device of example 1 wherein the one or more at least partially embedded sensors comprise one or more of an airflow sensor, a top or a bottom member alignment sensor, a head position sensor, an acid reflux sensor, a pH sensor, an H. pylori sensor, a temporomandibular joint stress sensor, or a dry mouth sensor.

Example 21 includes the oral therapy device of example 1 wherein the bottom member further includes a blood oxygen concentration sensor, a body temperature sensor, a vibration sensor or a snoring sensor.

Example 22 includes the oral therapy device of example 1 wherein a data bus is physically and electrically coupled to the one or more sensors, and wherein a first portion of the data bus is embedded within the top member, and wherein a second portion of the data bus is embedded within the bottom member.

Example 23 includes the oral therapy device of example 22 wherein the first portion and the second portion of the data bus are coupled to each other by an umbilical connector, wherein the umbilical connector is in a region that is between the top member and the bottom member.

Example 24 includes the oral therapy device of example 15 wherein an electrical bus is physically and electrically coupled to the one or more actuators, and wherein a first portion of the electrical bus is embedded within the top member, and wherein a second portion of the electrical bus is embedded within the bottom member.

Example 25 includes the oral therapy device of example 24 wherein the first portion and the second portion of the electrical bus are coupled to each other by an umbilical connector, wherein the umbilical connector is in a region that is between the top member and the bottom member.

Example 26 includes the oral therapy device of example 15 wherein the actuators comprise at least one a motor, a fan, or an electrode at least partially embedded within the oral therapy device.

Example 27 is an oral therapy device comprising:

a top member, wherein the top member fits over at least a portion of the upper teeth within an oral cavity of a user, a bottom member, wherein the bottom member fits over at least a portion of the bottom teeth within the oral cavity of the user, a coupling structure physically joining a portion of the top member to a portion of the bottom member, a light emitter coupled to and extended from a first portion of the bottom member, and a light detector coupled to and extended from a second portion of the bottom member, wherein the second portion is opposite the first portion.

Example 28 includes the oral therapy device of example 27 wherein the top member further includes at least one of an embedded microprocessor, storage memory or a battery.

Example 29 includes the oral therapy device of example 28 further including one or more sensors within the top member, wherein the one or more sensors comprises at least one of a vibration sensor, a snoring sensor, an airflow sensor, an orientation sensor, or a pH sensor.

Example 30 includes the oral therapy device of example 29 wherein the one or more sensors is communicatively coupled with the microprocessor.

Example 31 includes the oral therapy device of example 30 wherein the microprocessor is capable of receiving a signal from the one or more sensors, and wherein the microprocessor is capable of checking a target value for the one or more sensors.

Example 32 includes the oral therapy device of example 27 further including a sleeve on at least one of the light emitter or the light detector, wherein the sleeve comprises a body temperature sensor.

Example 33 includes the oral therapy device of example 27 wherein the bottom member further includes an electrical stimulus apparatus.

Example 34 includes the oral therapy device of example 33 wherein the electrical stimulus apparatus is capable of increasing the cross-sectional area of the airway.

Example 35 includes the oral therapy device of example 27 wherein the bottom member further includes a vibrational stimulus apparatus.

Example 36 includes the oral therapy device of example 27 wherein the vibrational stimulus apparatus is capable of increasing the cross-sectional area of the airway.

Example 37 includes the oral therapy device of example 27 wherein the bottom member further includes a mandibular positioning drive (MPD), wherein the MPD is at least partially embedded within the bottom member, and wherein the MPD is capable of moving the bottom member from a first position to a second position.

Example 38 includes the oral therapy device of example 28 wherein the microprocessor is capable of sending a signal to the MPD, and wherein the MPD is capable of moving a position of the bottom member.

Example 39 is an oral therapy device comprising:

a top member, wherein the top member fits over at least a portion of the upper teeth within an oral cavity of a user, a bottom member, wherein the bottom member fits over at least a portion of the bottom teeth within the oral cavity of the user; a coupling structure physically joining a portion of the top member to a portion of the bottom member; a first stimulation structure on a first portion of the bottom member; and a second stimulation structure on a second portion of the bottom member, wherein the second stimulation structure is opposite the first stimulation structure.

Example 40 includes the oral therapy device of example 39 wherein the first stimulation structure comprises a first pair of electrodes on the first portion of the bottom member, and the second stimulation structure comprises a second pair of electrodes on the second portion of the bottom member

Example 41 includes the oral therapy device of example 39 wherein the first pair and the second pair of electrodes are capable of providing an electrical stimulus to the tongue of the user.

Example 42 includes the oral therapy device of example 39 further comprising a band coupled between a third portion of the bottom member and a fourth portion of the bottom member, wherein the band is capable of providing a vibrational stimulus to a tongue of a human user.

Example 43 includes the oral therapy device of example 39 wherein the top member comprises one or more sensors.

Example 44 includes the oral therapy device of example 39 wherein the bottom member further includes a mandibular positioning drive (MPD), wherein the MPD is at least partially embedded within the bottom member, wherein the MPD is capable of moving the bottom member from a first position to a second position.

Example 4 includes the oral therapy device of example 39 wherein the top member comprises at least one of an embedded microprocessor, storage memory or a battery.

Example 46 includes the oral therapy device of example 45 wherein the one or more sensors are communicatively coupled with the microprocessor.

Example 47 is an oral therapy device comprising:

a top member, wherein the top member fits over at least a portion of the upper teeth within an oral cavity of a user, a bottom member, wherein the bottom member fits over at least a portion of the bottom teeth within the oral cavity of the user; a coupling structure physically joining a portion of the top member to a portion of the bottom member; and a sound generator structure at least partially embedded within at least one of the bottom member or the top member.

Example 48 includes the oral therapy device of example 47 wherein the top member comprises at least one embedded sensor.

Example 49 includes the oral therapy device of example 47 wherein the bottom member further includes an electrical stimulus apparatus.

Example 50 includes the oral therapy device of example 47 wherein the bottom member further includes a mandibular positioning drive (MPD), wherein the MPD is at least partially embedded within the bottom member, wherein the MPD is capable of moving the bottom member from a first position to a second position.

Example 51 includes the oral therapy device of example 47 further comprising a band coupled between a first portion of the bottom member and a second portion of the bottom member, wherein the band is capable of providing a vibrational stimulus to a tongue of the user.

Example 52 includes the oral therapy device of example 47 wherein the top member further includes at least one of an embedded microprocessor, storage memory or a battery.

Example 53 includes the oral therapy device of example 48 wherein the one or more sensors are communicatively coupled with the microprocessor.

Example 54 is an oral therapy device comprising:

a top member, wherein the top member fits over at least a portion of the upper teeth within an oral cavity of a user; a bottom member, wherein the bottom member fits over at least a portion of the bottom teeth within the oral cavity of the user; and a coupling structure physically joining a portion of the top member to a portion of the bottom member; and an airflow tube fully embedded within the top member.

Example 55 includes the oral therapy device of example 54 wherein the top member further includes at least one embedded sensor.

Example 56 includes the oral therapy device of example 54 wherein the bottom member includes an electrical stimulus apparatus.

Example 57 includes the oral therapy device of example 54 wherein the airflow tube is coupled to a fan that is fully embedded within the top member.

Example 58 includes the oral therapy device of example 57 wherein the fan is capable of increasing a cross-sectional area of an airway of the user.

Example 59 includes the oral therapy device of example 54 wherein the bottom member further includes a mandibular positioning drive (MPD), wherein the MPD is at least partially embedded within the bottom member, wherein the MPD is capable of moving the bottom member from a first position to a second position.

Example 60 includes the oral therapy device of example 54 further comprising a band coupled between a first portion of the bottom member and a second portion of the bottom member, wherein the band is capable of providing a vibrational stimulus to a tongue of a human user.

Example 61 includes the oral therapy device of example 55 wherein the top member further comprises at least one of an embedded microprocessor, storage memory or a battery.

Example 62 includes the oral therapy device of example 61 wherein the one or more sensors are communicatively coupled with the microprocessor.

Example 63 is a sleep enhancement system comprising:

a top member, wherein the top member fits over at least a portion of the upper teeth within an oral cavity of a user, and wherein the top member includes: at least one or more partially embedded sensors; a communication device capable of sending and receiving data; a bottom member, wherein the bottom member fits over at least a portion of the bottom teeth within the oral cavity of the user, and wherein the bottom member includes at least one of an electrical stimulus apparatus or a vibrational stimulus apparatus; and a base station communicatively coupled to the communication device, wherein the base station is capable of sending and receiving data.

Example 64 includes the system of example 63 wherein a mandibular positioning drive (MPD) is coupled to a portion of the top member and is coupled to a portion of the bottom member, and is capable of producing a displacement in a mandibular region of the user.

Example 65 includes the system of example 63 wherein the communication device is capable of receiving data from the one or more sensors.

Example 66 includes the system of example 64 wherein the MPD is capable of receiving data from the communication device.

Example 67 includes the system of example 63 wherein the base station is capable of receiving data from the at least one sensor.

Example 68 includes the system of example 63, wherein the base station is capable of storing historical data from the one or more sensors.

Example 69 includes the system of example 63 wherein the base station includes a sanitizing apparatus, wherein the sanitizing apparatus is capable of sanitizing the oral therapy device.

Example 70 is a sleep enhancement system comprising:

a top member, wherein the top member fits over at least a portion of the upper teeth within an oral cavity of a user, wherein the top member comprises at least one at least partially embedded sensor, and further comprises a microprocessor capable of sending and receiving data; a bottom member, wherein the bottom member fits over at least a portion of the bottom teeth within the oral cavity of the user, and wherein the bottom member comprises at least one of an electrical or a vibrational stimulus apparatus; a base station communicatively coupled to the microprocessor, wherein the base station is capable of sending and receiving data to and from the microprocessor; and an OASIS system protocol communicatively coupled to the base station.

Example 71 includes the system of example 70 wherein a mandibular positioning drive (MPD) is coupled to a portion of the top member and is coupled to a portion of the bottom member, and is capable of producing a displacement in a mandibular region of the user.

Example 72 includes the system of example 70 wherein the microprocessor is capable of receiving data from the one or more sensors.

Example 73 includes the system of example 70 wherein the base station comprises a tethering connector that is capable of physically coupling the oral therapy device to the base station.

Example 74 includes the system of example 70 wherein the base station comprises a compartment to receive the oral therapy device, wherein the compartment is capable of delivering UV radiation to clean the oral therapy device.

Example 75 includes the system of example 70 wherein the base station comprises one or more electronic interface ports capable of interfacing with one or more collaborative sensors.

Example 76 includes the system of example 75 wherein the one or more collaborative sensors comprise one or more of a muscle tension sensor, a rapid eye movement sensor, a pulse oximeter sensor, a chest effort sensor or a brain wave activity sensor.

Example 77 includes the system of example 70 wherein the microprocessor is communicatively coupled to one or more collaborative devices, wherein the collaborative devices comprise one or more of CPAP devices, smart beds, light-emitting/dampening therapy devices.

Example 78 includes the system of example 77 wherein the microprocessor is capable of activating the one or more collaborative devices.

Example 79 includes the system of example 70 wherein the base station comprises logic to process sensor data to generate oral therapy protocols for a user.

Example 80 includes the system of example 70 wherein the microprocessor is capable of receiving commands from a remote-control unit.

Example 81 includes the system of example 80 wherein the remote-control unit is capable of sending a request to the microprocessor to adjust a current therapy protocol.

Example 82 includes the system of example 70 wherein the OASIS system protocol comprises logic to communicate with a cloud system, wherein the cloud system comprises data, and wherein the data comprises one or more therapy protocols from one or more additional users.

Example 83 includes the system of example 82 wherein the one or more therapy protocols comprise historical therapy protocol data from the one or more additional users.

Example 84 includes the system of example 82 wherein the OASIS system protocol includes one or more individual data repository modules (IDRM) capable of storing historical data from the user and the one or more additional users.

Example 85 includes the system of example 82 wherein the user and the one or more additional users each have an IDRM that is capable of storing user-specific demographic data.

Example 86 includes the system of example 84 wherein the individual IDRM of the user and the one or more additional users is capable of interfacing with and accessing data from one or more health care systems.

Example 87 includes the system of example 84 wherein the IDRM is capable of receiving data from online interviews and is capable of receiving direct input from the user and the one or more additional users.

Example 88 includes the system of example 70 wherein the OASIS system protocol includes an aggregate population data repository (APDR).

Example 89 includes the system of example 88 wherein the APDR is capable of receiving and storing anonymous user data, wherein the anonymous user data includes scrubbed data from the one or more IDRM.

Example 90 includes the system of example 82 wherein the cloud system includes a heuristic protocol development module (HPDM).

Example 91 includes the system of example 90 wherein the HPDM is capable of receiving aggregate population data from the APDR.

Example 92 includes the system of example 88 wherein the HPDM is capable of generating multiple general population profiles (GPPs), and is capable of storing the GPP's within the HPDM.

Example 93 includes the system of example 70 wherein the OASIS system protocol includes a heuristic user-specific development module (HUSDM).

Example 94 includes the system of example 93 wherein the HUSDM is capable of identifying the GPP most closely associated with a specific individual user, wherein the specific individual user is selected from the user or the one or more additional users.

Example 95 includes the system of example 94 wherein the HUSDM is capable of determining an appropriate therapy protocol revision for the specific individual user.

Example 96 includes the system of example 93 wherein the HUSDM is capable of communicating with the base station, and is capable of communicating with base stations of the one or more additional users.

Example 97 is a method of monitoring sleep parameters, comprising:

receiving data from an oral therapy device, wherein the oral therapy device comprises: a top member, wherein the top member fits over at least a portion of the upper teeth within an oral cavity of a user, wherein the top member comprises at least one partially embedded sensor and a microprocessor to receive the data from the at least one embedded sensor, and wherein the microprocessor is capable of receiving and sending data; a bottom member, wherein the bottom member fits over at least a portion of the bottom teeth within the oral cavity of the user, and wherein the bottom member comprises at least one of an electrical or a vibrational stimulus apparatus; wherein the bottom member is located in a first position, and wherein a first distance is between a portion of the base of the tongue and a portion of the posterior pharynx of the user; and moving the bottom member to a second position in response to receiving a signal from the microprocessor.

Example 98 includes the method of example 97 wherein moving the bottom member to a second position further comprises, wherein the bottom member is moved in response to an analysis of sensor data.

Example 99 includes the method of example 97 further comprising wherein the bottom member includes a mandibular positioning drive (MPD) that is coupled to a microprocessor that is located in the top member, wherein the MPD advances and retracts the bottom member.

Example 100 includes the method of example 97 wherein the microprocessor analyzes the sensor data, and moves the bottom member in response to the analyzed sensor data.

Example 101 includes the method of example 99 further comprising wherein the MPD moves the bottom member forward or backward.

Example 102 includes the method of example 101 wherein the movement of the bottom member retracts or advances the mandible of the user.

Example 103 includes the method of example 97 further comprising wherein a cross sectional area of an airway of the user is increased.

Example 104 includes the method of example 97 further comprising a base station to receive sensor data from the microprocessor.

Example 105 includes the method of example 104 further comprising wherein the base station stores historical sensor data.

Example 106 includes the method of example 105 wherein the historical sensor data is transmitted to a cloud server.

Example 107 includes the method of example 105 further comprising wherein the historical sensor data is used to monitor the usage of the sleep optimization device.

Example 108 includes the method of example 99 further comprising optimizing a first position and a second position of the MPD to minimize a displacement distance of an articular disk of the temporomandibular joint of the user, and moving the displacement distance a distance forward and backward during a time period to reduce stress on the articular disk.

Example 109 is a method of monitoring sleep parameters, comprising:

receiving data from one or more sensors coupled to an oral therapy device, wherein the oral therapy device comprises: a top member, wherein the top member fits over at least a portion of the upper teeth within an oral cavity of a user, wherein the top member comprises on or more top member sensors; a bottom member, wherein the bottom member fits over at least a portion of the bottom teeth within the oral cavity of the user, and wherein the bottom member comprises one or more bottom member sensors and a mandibular positioning device (MPD); determining whether the data is within a target range for each of the one or more sensors, wherein a microprocessor checks the sensor target value within the data received for each of the one or more sensors; analyzing the data from the one or more top and bottom sensors to generate an individualized user therapy protocol, wherein the individualized user therapy protocol comprises one or more therapies; and sending a signal from the microprocessor to one or more therapy actuator location within the oral therapy device in response to receiving data from the one or more sensors.

Example 110 includes the method of example 109 wherein the data from each individual sensor of the one or more top member sensors and the one or more bottom member sensors is stored in an individual memory location corresponding with each individual sensor, wherein the individual memory locations reside within the microprocessor.

Example 111 includes the method of example 110 wherein the individual memory locations comprise a history profile for data from each of the one or more sensors.

Example 112 includes the method of example 111 wherein one or more history profiles are analyzed by one or more Sleep Health Opportunity Monitors, and are analyzed by a Sleep State Bias Monitor.

Example 113 includes the method of example 112 comprising sending one or more sensor history profiles to an Integration Analysis Module when sensor target values are out of range.

Example 114 includes the method of example 109 wherein therapy modification instructions are generated and sent to one or more activation profile management modules.

Example 115 includes the method of 110 wherein an activation profile management module sends therapy instructions to one or more therapy actuators that are located within at least one of the top and the bottom members of the oral therapy device.

Example 116 includes the method of 109 wherein the bottom member is located in a first position, and wherein a first distance is between a portion of the base of the tongue and a portion of the posterior pharynx of the user, and wherein the bottom member is moved to a second position in response to receiving a signal from the microprocessor, wherein a second distance is between the portion of the base of the tongue and the posterior pharynx of the user.

Example 117 includes the method of example 105 wherein the microprocessor is embedded within top member.

Example 118 includes the method of example 109 wherein the one or more sensors of the top and bottom sensors comprise at least one of an auditory sensor, an airflow sensor, a vibration sensor, an orientation sensor, or a pH sensor.

Example 119 includes the method of example 109 wherein the one or more sensors are communicatively coupled with the microprocessor.

Example 120 includes the method of example 109 wherein the microprocessor is capable of receiving a signal from the at least one sensor, and wherein the microprocessor is capable of checking a target value for the at least one sensor.

Example 12 includes the method of example 109 wherein the microprocessor is capable of sending a signal to the MPD, wherein the MPD is capable of moving the bottom member.

Example 122 includes the method of example 109 further comprising sending the data received from the one or more top and bottom sensors to a base station.

Example 123 includes the method of example 122 wherein the data received from the one or more top and bottom sensors is temporarily stored in a device data management location that resides within the microprocessor.

Example 124 includes the method of example 123 wherein the data received from the one or more top and bottom sensors is analyzed for therapeutic optimization and further refines the therapeutic protocol for an individual user, and then sends optimized therapeutic instructions to the microprocessor.

Example 125 includes the method of example 124, wherein the microprocessor sends the optimized therapeutic instructions to the one or more actuators.

Example 126 includes the method of example 120 further comprising further comprising archived data from the base station to an OASIS cloud system.

Example 127 is a system to optimize sleep parameters comprising:

an oral therapy device, the oral therapy device comprising: a top member comprising one or more sensors; a bottom member comprising a mandibular positioning drive; a microprocessor to receive data from the one or more sensors; a base station to receive data from the microprocessor; and an OSIS system protocol to receive data from the base station.

Example 128 is a method to optimize sleep parameters comprising:

sending sensor data from one or more sensors of an oral therapy device to a data management module located within a microprocessor embedded within the oral therapy device, the oral therapy device comprising: a top member comprising the or more sensors; a bottom member comprising a mandibular positioning drive; converting the sensor data into digitized sensor data; sending the digitized sensor data to a base station; analyzing the digitized sensor data and generating a first therapy protocol from the digitized sensor data; sending the first therapy protocol to the microprocessor when the first therapy protocol is required by a user; sending the first therapy protocol to an OASIS system; and comparing the first therapy protocol to one or more additional therapy protocols; generating a second therapy protocol; and sending the second therapy protocol to the microprocessor.

Example 129 includes the method of example 128 wherein data from each individual sensor is stored in an individual memory location corresponding with each individual sensor, wherein individual memory locations reside within the microprocessor.

Example 130 includes the method of example 129 wherein the individual memory locations comprise a history profile for data from each of the one or more sensors.

Example 131 includes the method of example 130 wherein the history profile is analyzed by one or more Sleep Health opportunity Monitors and are analyzed by a Sleep State Bias Monitor.

Example 132 includes the method of example 130 further comprising sending the history profile to an Integration Analysis Module when a sensor target value is out of range.

Example 133 includes the method of example 132 wherein therapy modification instructions are generated and sent to one or more activation profile management modules.

Example 134 includes the method of example 133 wherein an activation profile management module sends therapy instructions to one or more therapy actuators located within at least one of the top and bottom members of the oral therapy device.

Example 135 includes the method of example 128 wherein the bottom member is located in a first position, and wherein a first distance is between a portion of the base of the tongue and a portion of the posterior pharynx of the user, and wherein moving the bottom member is moved to a second position in response to receiving a signal from the microprocessor, wherein a second distance is between the portion of the base of the tongue and the posterior pharynx of the user.

Example 136 includes the method of example 128 wherein the microprocessor is embedded within the top member.

Example 137 includes the method of example 128 wherein the one or more sensors comprise at least one of an auditory sensor, an airflow sensor, a vibration sensor, an orientation sensor, or a pH sensor.

Example 138 includes the method of example 128 wherein the one or more sensors are communicatively coupled with the microprocessor.

Example 139 includes the method of example 128 wherein the microprocessor receives a signal from the at least one sensor, and wherein the microprocessor checks a target value for the one or more sensors.

Example 140 includes the method of example 128 wherein the microprocessor sends a signal to a mandibular positioning drive, and wherein the MPD moves the bottom member.

Example 141 includes the method of example 128 further comprising sending the data received from the one or more sensors to a base station.

Example 142 includes the method of example 128 wherein the data received from the one or more sensors is temporarily stored in a device data management location that resides within the microprocessor.

Example 143 includes the method of example 128 further comprising analyzing the data received from the one or more sensors, optimizing the data to generate a therapeutic protocol for an individual user, and then sending the therapeutic protocol to the microprocessor.

Example 144 includes the method of example 143, further comprising sending the therapeutic protocol to the one or more actuators.

Example 145 includes the method of example 141 further comprising archiving the data from the base station to an OASIS cloud system.

Example 146 is a method to optimize sleep parameters comprising:

sending sensor data from one or more sensors of an oral therapy device from an individual user to a microprocessor embedded within the oral therapy device, the oral therapy device comprising: a top member comprising one or more sensors; and a bottom member comprising a mandibular positioning drive; analyzing the sensor data to determine out of range sensor target values; generating a therapy protocol from the analyzed sensor data; sending the therapy protocol to an OASIS system; and storing the therapy protocol, the out of range sensor target values, and the sensor data subsequent to the application of the therapy protocol within an individual data repository module (IDRM).

Example 147 includes the method of example 146 further including sending user-specific demographic data to the IDRM.

Example 148 includes the method of example 146 further including sending data from an individual user within the IDRM to an aggregate user population data repository (APDR), wherein the APDR contains data from additional users, wherein the additional users' data are connected to each other through an OASIS cloud.

Example 149 includes the method of example 148 further including sending the data from the APDR to a Heuristic Protocol Development Module (HPDM), wherein the HPDM identifies and updates multiple general population profiles (GPP) among the user and the additional users.

Example 150 includes the method of example 149 wherein the GPP groups are selected from the APDR, wherein the selection is chosen on the basis of age, sex, race, weight, or neck size.

Example 151 includes the method of example 150 comprising comparing the IDRM of an individual user within a specific GPP group to the IDRM's of other GPP group members, and optimizing the individual user's therapy protocol.

Example 152 includes the method of example 151 comprising performing an optimization of the individual user's therapy protocol within a Heuristic User Specific Development Module.

Example 153 includes the method of example 152 comprising sending the optimized user therapy protocol to the individual user's oral therapy device.

Example 154 includes the method of example 153 further comprising comparing a first therapy protocol to one or more additional therapy protocols; generating a second therapy protocol; and sending the second therapy protocol to the microprocessor.

Although the foregoing description has specified certain steps and materials that may be used in the methods of the embodiments, those skilled in the art will appreciate that many modifications and substitutions may be made. Accordingly, it is intended that all such modifications, alterations, substitutions and additions be considered to fall within the spirit and scope of the embodiments as defined by the appended claims. In addition, the Figures provided herein illustrate only portions of exemplary oral device structures and associated methods and systems that pertain to the practice of the embodiments. Thus the embodiments are not limited to the structures described herein.

What is claimed is:

1. An oral therapy device comprising:
a top member, wherein the top member fits over at least a portion of upper teeth within an oral cavity of a user, and wherein the top member comprises one or more at least partially embedded sensors;
a bottom member, wherein the bottom member fits over at least a portion of bottom teeth within the oral cavity of the user;
a coupling structure physically joining a portion of the top member to a portion of the bottom member; and
a mandibular positioning drive (MPD), wherein the MPD is at least partially embedded within the bottom member, and wherein the MPD is capable of moving the bottom member from a first position to a second position.

2. The oral therapy device of claim 1 wherein the bottom member further includes at least one of an electrical stimulus apparatus, a vibrational stimulus apparatus or a sound speaker.

3. The oral therapy device of claim 1 wherein the first position of the MPD comprises one of an advancement or a retraction of the mandible, wherein the second position of the MPD comprises one of an advancement or retraction of the mandible.

4. The oral therapy device of claim 1 wherein the first position and the second position of the MPD are capable of being optimized to minimize a displacement distance of the articular disk of the temporomandibular joint, and wherein the displacement distance comprises a distance forward and a distance backward during a time period.

5. The oral therapy device of claim 1 wherein the top member further includes at least one of an embedded microprocessor, a storage memory or a battery, and wherein the microprocessor is capable of sending a signal to the MPD, and wherein the MPD is capable of moving the bottom member.

6. The oral therapy device of claim 1 wherein the one or more sensors comprises at least one of an auditory sensor, an airflow sensor, a vibration sensor, an orientation sensor, or a pH sensor, wherein the one or more sensors is communicatively coupled with the microprocessor.

7. The oral therapy device of claim 6 wherein the microprocessor is capable of receiving a signal from the one or more sensors, and wherein the microprocessor is capable of checking a target value for the one or more sensors.

8. The oral therapy device structure of claim 6 wherein the microprocessor is communicatively coupled to one or more actuators that are at least partially embedded within the oral therapy device, and wherein an MPD actuator is physically coupled to the MPD, and wherein the one or more actuators include at least one of an electrical motor, a fan or an electrode.

9. The oral therapy device of claim 1 wherein an electrical stimulus apparatus is at least partially embedded within the bottom member.

10. The oral therapy device of claim 1 wherein the one or more at least partially embedded sensors comprise one or more of an airflow sensor, a top or a bottom member alignment sensor, a head position sensor, an acid reflux sensor, a pH sensor, an H. pylori sensor, a temporomandibular joint stress sensor, or a dry mouth sensor.

11. The oral therapy device of claim 1 wherein the bottom member further includes a blood oxygen concentration sensor, a body temperature sensor, a vibration sensor or a snoring sensor.

12. The oral therapy device of claim 8 wherein an electrical bus is physically and electrically coupled to the one or more actuators, and wherein a first portion of the electrical bus is embedded within the top member, and wherein a second portion of the electrical bus is embedded within the bottom member.

13. The oral therapy device of claim 12 wherein the first portion and the second portion of the electrical bus are coupled to each other by an umbilical connector, wherein the umbilical connector is in a region that is between the top member and the bottom member.

14. The oral therapy device of claim 1 further comprising:
a light emitter coupled to and extended from a first portion of the bottom member;
a light detector coupled to and extended from a second portion of the bottom member, wherein the second portion is opposite the first portion; and
a sleeve on at least one of the light emitter or the light detector, wherein the sleeve comprises a body temperature sensor.

15. The oral therapy device of claim 1 wherein the bottom member further includes an electrical stimulus apparatus, wherein the electrical stimulus apparatus is capable of increasing the cross-sectional area of the airway.

16. The oral therapy device of claim 1 wherein the bottom member further includes a vibrational stimulus apparatus, wherein the vibrational stimulus apparatus is capable of increasing the cross-sectional area of the airway.

17. The oral therapy device of claim 1 further comprising:
a first stimulation structure on a first portion of the bottom member;
a second stimulation structure on a second portion of the bottom member, wherein the second stimulation structure is opposite the first stimulation structure, and wherein the first stimulation structure comprises a first pair of electrodes on the first portion of the bottom member, and the second stimulation structure comprises a second pair of electrodes on the second portion of the bottom member.

18. The oral therapy device of claim 17 wherein the first pair and the second pair of electrodes are capable of providing an electrical stimulus to the tongue of the user, and wherein a band coupled between a third portion of the bottom member and a fourth portion of the bottom member is capable of providing a vibrational stimulus to a tongue of a human user.

19. The oral therapy device of claim 1 further comprising a sound generator structure at least partially embedded within at least one of the bottom member or the top member.

20. The oral therapy device of claim 1 further comprising an airflow tube fully embedded within the top member, wherein the airflow tube is coupled to a fan that is fully embedded within the top member, wherein the fan is capable of increasing a cross-sectional area of an airway of the user.

21. A sleep enhancement system comprising:
a top member, wherein the top member fits over at least a portion of the upper teeth within an oral cavity of a user, and wherein the top member includes:
at least one or more partially embedded sensors;
a communication device capable of sending and receiving data;
a bottom member, wherein the bottom member fits over at least a portion of the bottom teeth within the oral cavity of the user, and wherein the bottom member includes at least one of an electrical stimulus apparatus or a vibrational stimulus apparatus;
a base station communicatively coupled to the communication device, wherein the base station is capable of sending and receiving data; and
an Obstructive Apnea Support and Information System (OASIS) system protocol communicatively coupled to the base station.

22. The system of claim 21 wherein a mandibular positioning drive (MPD) is coupled to a portion of the top member and is coupled to a portion of the bottom member, and is capable of producing a displacement in a mandibular region of the user, wherein the MPD is capable of receiving data from the communication device.

23. The system of claim 21 wherein the base station is capable of receiving data from the at least one sensor, wherein the base station is capable of storing historical data from the one or more sensors.

24. The system of claim 21 wherein the base station includes at least one of a sanitizing apparatus, wherein the sanitizing apparatus is capable of sanitizing the oral therapy device, a tethering connector that is communicatively coupled to the oral therapy device, or a compartment to receive the oral therapy device, wherein the compartment is capable of delivering UV radiation to clean the oral therapy device.

25. The system of claim 21 wherein a microprocessor is coupled to the top member and is communicatively coupled to the one or more sensors.

26. The system of claim 21 wherein the base station comprises one or more electronic interface ports capable of interfacing with one or more collaborative sensors.

27. The system of claim 21 wherein the one or more collaborative sensors comprise one or more of a muscle tension sensor, a rapid eye movement sensor, a pulse oximeter sensor, a chest effort sensor or a brain wave activity sensor, wherein the microprocessor is communicatively coupled to one or more collaborative devices, and wherein the one or more collaborative devices comprise one or more of a continuous positive airway pressure (CPAP) device, a smart bed, a light-emitting therapy device or a dampening therapy device.

28. The system of claim 21 wherein the OASIS system protocol comprises logic to communicate with a cloud system, wherein the cloud system comprises data, and wherein the data comprises one or more therapy protocols from one or more additional users, wherein the one or more therapy protocols comprise historical therapy protocol data from the one or more additional users.

29. The system of claim 21 wherein the OASIS system protocol includes one or more individual data repository modules (IDRM) capable of storing historical data from the user and the one or more additional users, wherein the user and the one or more additional users each have an IDRM that is capable of storing user-specific demographic data, and wherein the individual IDRM of the user and the one or more additional users is capable of interfacing with and accessing data from one or more health care systems.

30. The system of claim 29 wherein the IDRM is capable of receiving data from online interviews and is capable of receiving direct input from the user and the one or more additional users.

31. The system of claim 30 wherein the OASIS system protocol includes an aggregate population data repository (APDR), wherein the APDR is capable of receiving and storing anonymous user data, wherein the anonymous user data includes scrubbed data from the one or more IDRM.

32. The system of claim 31 wherein the cloud system includes a heuristic protocol development module (HPDM), wherein the HPDM is capable of receiving aggregate population data from the APDR, wherein the HPDM is capable of generating multiple general population profiles (GPPs), and is capable of storing the GPP's within the HPDM.

33. The system of claim 32 wherein the OASIS system protocol includes a heuristic user-specific development module (HUSDM), wherein the HUSDM is capable of identifying the GPP most closely associated with a specific individual user, wherein the specific individual user is selected from the user or the one or more additional users.

34. The system of claim 33 wherein the HUSDM is capable of determining an appropriate therapy protocol revision for the specific individual user, wherein the HUSDM is capable of communicating with the base station, and is capable of communicating with base stations of the one or more additional users.

35. A method of monitoring sleep parameters, comprising:
receiving data from an oral therapy device, wherein the oral therapy device comprises:
a top member, wherein the top member fits over at least a portion of the upper teeth within an oral cavity of a user, wherein the top member comprises at least one partially embedded sensor and a microprocessor to receive the data from the at least one embedded sensor, and wherein the microprocessor is capable of receiving and sending data;
a bottom member, wherein the bottom member fits over at least a portion of the bottom teeth within the oral cavity of the user, and wherein the bottom member comprises at least one of an electrical or a vibrational stimulus apparatus;
wherein the bottom member is located in a first position, and wherein a first distance is between a portion of the base of the tongue and a portion of the posterior pharynx of the user; and
moving the bottom member to a second position in response to receiving a signal from the microprocessor.

36. The method of claim 35 wherein moving the bottom member to a second position further comprises, wherein the bottom member is moved in response to an analysis of sensor data.

37. The method of claim 35 further comprising wherein the bottom member includes a mandibular positioning drive (MPD) that is coupled to a microprocessor that is located in the top member, wherein the MPD advances and retracts the bottom member.

38. The method of claim 35 wherein the microprocessor analyzes the sensor data, and moves the bottom member in response to the analyzed sensor data, wherein the movement of the bottom member retracts or advances the mandible of the user wherein a cross sectional area of the airway of the user is increased.

39. The method of claim 37 further comprising a base station to receive sensor data from the microprocessor, wherein the base station stores historical sensor data, wherein the historical sensor data is transmitted to a cloud server, and wherein the historical sensor data is used to monitor the usage of the oral therapy device.

40. The method of claim 39 further comprising:
determining whether the historical data is within a target range for each of the one or more sensors, wherein a microprocessor checks the sensor target value within the data received for each of the one or more sensors;
analyzing the historical data from at least one of the top sensor or the bottom sensor to generate an individualized user therapy protocol, wherein the individualized user therapy protocol comprises one or more therapies; and
sending a signal from the microprocessor to one or more therapy actuator location within the oral therapy device in response to receiving data from the one or more sensors.

41. The method of claim 40 wherein data from each individual sensor of the one or more top member sensors and the one or more bottom member sensors is stored in an individual memory location corresponding to each individual sensor, wherein the individual memory locations reside within the microprocessor, and wherein the individual memory locations comprise a history profile for the data from each of the one or more sensors.

42. The method of claim 41 further comprising analyzing the one or more history profiles by one or more Sleep Health Opportunity Monitors, and analyzing the one or more Sleep Health Opportunity Monitors by a Sleep State Bias Monitor, and sending one or more sensor history profiles to an Integration Analysis Module when sensor target values are out of range.

43. The method of claim 42 further comprising generating therapy modification instructions and sending one or more activation profile management modules to one or more therapy actuators that are located within at least one of the top and the bottom members of the oral therapy device.

44. The method of claim 35 comprising receiving a signal from the at least one sensor, checking a target value for the at least one sensor, sending a signal to the MPD, and moving the bottom member.

45. The method of claim 35 further comprising sending the data received from the at least one sensor to a base station.

46. The method of claim 35 further comprising analyzing the data received from the at least one sensor for therapeutic optimization and further refining the therapeutic protocol for an individual user, and then sending the optimized therapeutic instructions to one or more actuators.

47. The method of claim 46 further comprising archiving data from the base station to an Obstructive Apnea Support and Information System (OASIS) cloud system.

48. A method to optimize sleep parameters comprising:
sending sensor data from one or more sensors of an oral therapy device to a data management module located within a microprocessor embedded within the oral therapy device, the oral therapy device comprising:
a top member comprising one or more sensors;
a bottom member comprising a mandibular positioning drive;
converting the sensor data into digitized sensor data;
sending the digitized sensor data to a base station;
analyzing the digitized sensor data and generating a first therapy protocol from the digitized sensor data;
sending the first therapy protocol to an Obstructive Apnea Support and Information System (OASIS) system when the first therapy protocol is required by a user;
comparing the first therapy protocol to one or more additional therapy protocols;
generating a second therapy protocol; and
sending the second therapy protocol to the OASIS system.

49. The method of claim 48 further including sending user-specific demographic data to the OASIS system, wherein additional users' data is accessible in the OASIS system, and wherein an aggregate user population data repository (APDR) contains data from additional users.

50. The method of claim 49 further including sending data from an individual user within the IDRM to the APDR, wherein the APDR contains data from additional users, wherein a Heuristic Protocol Development Module (HPDM), identifies and updates multiple general population profiles (GPP) among the user and the additional users, and wherein the GPP profiles are selected from the APDR, wherein the selection is chosen on the basis of age, sex, race, weight, or neck size.

51. The method of claim 48 comprising comparing at least one of the first therapy protocol or the second therapy protocol with the GPP profiles, and then performing an optimization of at least one of the first therapy protocol or the second therapy protocol with the individual user's therapy protocol.

52. The method of claim 51 comprising sending at least one of the optimized first therapy protocol or the optimized second therapy protocol to the oral therapy device.
